(19) United States
(12) Patent Application Publication
(10) Pub. No.: US 2024/0103016 A1
(43) Pub. Date: Mar. 28, 2024

(54) METHODS OF DETECTING PAPP-A AND RELATED METHODS FOR GESTATIONAL AGE ASSESSMENT

(71) Applicants: Gynuity Health Projects, Inc., New York, NY (US); The Board of Trustees of University of Illinois, Urbana, IL (US)

(72) Inventors: Irina BUHIMSCHI, Chicago, IL (US); Laura FRYE, New York, NY (US); Elizabeth RAYMOND, New York, NY (US); Beverly WINIKOFF, New York, NY (US)

(73) Assignees: Gynuity Health Projects, Inc., New York, NY (US); The Board of Trustees of University of Illinois, Urbana, IL (US)

(21) Appl. No.: 18/266,149

(22) PCT Filed: Dec. 8, 2021
(86) PCT No.: PCT/US2021/062483

§ 371 Date: Jun. 8, 2023

Related U.S. Application Data
(60) Provisional application No. 63/123,467, filed on Dec. 9, 2020.

(51) Int. Cl. G01N 33/68; G01N 33/543

(52) U.S. Cl. CPC G01N 33/689; G01N 33/54387; G01N 2470/06; G01N 2800/52

(57) ABSTRACT

Provided herein are methods, devices, kits, and articles of manufacture for detecting pregnancy-associated plasma protein-A (PAPP-A) in a biological sample, for instance a whole blood or serum sample obtained from a pregnant subject, and estimating gestational age (GA) based on the detection of PAPP-A, such that clinical or personal decisions informed by GA can be made without ultrasound.

### CROSS-REFERENCE TO RELATED APPLICATIONS

This application claims priority from U.S. provisional application No. 63/123,467 filed Dec. 9, 2020, entitled “METHODS OF DETECTING PAPP-A AND RELATED METHODS FOR GESTATIONAL AGE ASSESSMENT,” the contents of which are incorporated by reference in their entirety.

### FIELD

The present disclosure relates to methods, devices, kits, and articles of manufacture for detecting pregnancy-associated plasma protein-A (PAPP-A) in a biological sample, for instance a whole blood or serum sample obtained from a pregnant subject, and estimating gestational age (GA) based on the detection of PAPP-A, such that clinical or personal decisions informed by GA can be made without ultrasound.

### BACKGROUND

Accurate knowledge of gestational age (GA) is essential for all aspects of prenatal care. Because assessment based on menstrual history and examination of uterine size is often unreliable, clinicians currently routinely use ultrasound to estimate GA. However, ultrasound is expensive, must be performed by a trained technician, and is generally provided only in specialized medical facilities. Thus, there remains a need for reagents, devices, and methods for detecting PAPP-A in samples from pregnant subjects and estimating the GA of a pregnancy. In particular, reagents, devices, and methods that provide a cheaper, more accessible GA estimation tool could be highly beneficial, particularly in low resource settings or when providing care remotely through telemedicine. Provided herein are embodiments that meet such needs.

### SUMMARY

Provided herein in some embodiments is a method of detecting PAPP-A in a biological sample from a pregnant subject, comprising: (a) preparing a test sample from a pregnant female subject, the preparing comprising: (i) obtaining a biological sample from the pregnant female subject, wherein the obtained biological sample is a whole blood, serum, or plasma sample and wherein the obtained biological sample has a volume between or between about 0.5 μL and 10 inclusive; and (ii) diluting the volume of the obtained biological sample in a sample diluent between or between about 2-fold and 100-fold, inclusive, thereby preparing the test sample; and (b) detecting in the test sample one or more PAPP-A proteoforms using an immunoassay, said one or more PAPP-A proteoforms comprising homodimeric PAPP-A and/or a heterotetrameric PAPP-A/proMBP complex, wherein the immunoassay comprises: (i) contacting the test sample with an antibody capable of specifically binding to (1) homodimeric PAPP-A and (2) the heterotetrameric PAPP-A/proMBP complex under conditions to form a complex comprising the antibody and the one or more PAPP-A proteoforms; and (ii) detecting the complex comprising the antibody and the one or more PAPP-A proteoforms.

In some embodiments, when the biological sample is obtained, the gestational age (GA) of the pregnant female subject is suspected to be between or between about 5 weeks and 40 weeks, between or between about 5 weeks and 30 weeks, or between or between about 5 weeks and 20 weeks, each inclusive. In some of any of the provided embodiments, when the biological sample is obtained, the gestational age (GA) of the pregnant female subject is suspected to be between or between about 5 weeks and 20 weeks, inclusive. In some embodiments, when the biological sample is obtained, the GA of the pregnant female subject is suspected to be between or between about 5 weeks and 15 weeks, inclusive. In some embodiments, when the biological sample is obtained, the GA of the pregnant female subject is suspected to be between or between about 5 weeks and 10 weeks, inclusive.

In some embodiments, when the biological sample is obtained, the GA of the pregnant female subject is suspected to be between or between about 35 days and 70 days, inclusive. In some embodiments, when the biological sample is obtained, the GA of the pregnant female subject is suspected to be between or between about 35 days and 69 days, inclusive. In some embodiments, when the biological sample is obtained, the GA of the pregnant female subject is suspected to be between or between about 35 days and 68 days, inclusive. In some embodiments, when the biological sample is obtained, the GA of the pregnant female subject is suspected to be between or between about 35 days and 67 days, inclusive. In some embodiments, when the biological sample is obtained, the GA of the pregnant female subject is suspected to be between or between about 35 days and 66 days, inclusive. In some embodiments, when the biological sample is obtained, the GA of the pregnant female subject is suspected to be between or between about 35 days and 65 days, inclusive. In some embodiments, when the biological sample is obtained, the GA of the pregnant female subject is suspected to be between or between about 35 days and 64 days, inclusive. In some embodiments, when the biological sample is obtained, the GA of the pregnant female subject is suspected to be between or between about 35 days and 63 days, inclusive.

In some of any of the provided embodiments, the immunoassay is a colorimetric assay.

In some of any of the provided embodiments, the immunoassay is a solid-phase immunoassay. In some of any of the provided embodiments, the antibody is immobilized on a solid support.

In some of any of the provided embodiments, the immunoassay is an Enzyme linked immunosorbent assay (ELISA). In some of any of the provided embodiments, the immunoassay is a sandwich ELISA.

In some of any of the provided embodiments, the immunoassay is a lateral flow assay.

In some of any of the provided embodiments, the antibody is capable of specifically binding to (1) homodimeric PAPP-A and (2) the heterotetrameric PAPP-A/proMBP complex equimolarly or about equimolarly.

In some of any of the provided embodiments, the antibody does not specifically bind to proMBP. In some of any of the provided embodiments, the antibody does not cross-react with proMBP. In some of any of the provided embodiments, the antibody does not cross-react with proMBP at up to twice the physiological concentrations of proMBP. In some of any of the provided embodiments, the antibody does not specifically bind to PAPP-A2. In some of any of the provided embodiments, the antibody does not cross-react with PAPP-A2. In some of any of the provided embodiments, the antibody does not cross-react with PAPP-A2 at up to twice the physiological concentrations of PAPP-A2. In some of any of the provided embodiments, the antibody does not specifically bind to MMP-9. In some of any of the provided embodiments, the antibody does not cross-react with MMP-9. In some of any of the provided embodiments, the antibody does not cross-react with MMP-9 at up to twice the physiological concentrations of MMP-9. In some of any of the provided embodiments, the physiological concentrations are with respect to serum concentration levels. In some of any of the provided embodiments, the physiological concentrations are with respect to serum concentration levels in pregnant subjects.

In some of any of the provided embodiments, the antibody specifically binds to a PAPP-A subunit of homodimeric PAPP-A. In some of any of the provided embodiments, the antibody specifically binds to a PAPP-A subunit of the heterotetrameric PAPP-A/proMBP complex. In some of any of the provided embodiments, the antibody specifically binds to a PAPP-A subunit of homodimeric PAPP-A and specifically binds to a PAPP-A subunit of the heterotetrameric PAPP-A/proMBP complex.

In some of any of the provided embodiments, the antibody does not bind the metal chelating region of PAPP-A.

In any of the provided embodiments, the antibody is a capture antibody and the complex is a first complex comprising the capture antibody and the one or more PAPP-A proteoforms, and wherein prior to the detecting, the immunoassay further comprises contacting the first complex comprising the capture antibody and the one or more PAPP-A proteoforms with a detection antibody capable of specifically binding to (1) homodimeric PAPP-A and (2) the heterotetrameric PAPP-A/proMBP complex under conditions to form a second complex comprising the capture antibody, the one or more PAPP-A proteoforms, and the detection antibody; and the detecting comprises detecting the second complex.

In some of any of the provided embodiments, the antibody is a detection antibody and the complex is a first complex comprising the detection antibody and the one or more PAPP-A proteoforms, and wherein prior to the detecting, the immunoassay further comprises contacting the first complex comprising the detection antibody and the one or more PAPP-A proteoforms with a capture antibody capable of specifically binding to (1) homodimeric PAPP-A and (2) the heterotetrameric PAPP-A/proMBP complex under conditions to form a second complex comprising the capture antibody, the one or more PAPP-A proteoforms, and the detection antibody; and the detecting comprises detecting the second complex.

In some of any of the provided embodiments, the detection antibody is conjugated to a detectable label capable of producing a detectable signal, and the detecting the second complex is by assessing the degree of the detectable signal produced by the detectable label. In some of any of the provided embodiments, the method further comprises determining the concentration of PAPP-A in the obtained biological sample by comparison of the degree of the detectable signal assessed from the test sample to a standard curve.

Also provided herein in some embodiments is a method of detecting PAPP-A in a biological sample from a pregnant subject, comprising: (a) preparing a test sample from a pregnant female subject, the preparing comprising: (i) obtaining a biological sample from the pregnant female subject, wherein the obtained biological sample is a whole blood, serum, or plasma sample and wherein the obtained biological sample has a volume between or between about 0.5 μL and 10 μL, inclusive; and (ii) diluting the volume of the obtained biological sample in a sample diluent between or between about 2-fold and 100-fold, inclusive, thereby preparing the test sample; and (b) detecting in the test sample one or more PAPP-A proteoforms using an immunoassay, said one or more PAPP-A proteoforms comprising homodimeric PAPP-A and/or a heterotetrameric PAPP-A/proMBP complex, wherein the immunoassay comprises: (i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and (ii) assessing the degree of the detectable signal produced by the detectable label.

In some embodiments, the contacting of the test sample with the capture antibody and the detection antibody is carried out simultaneously. In some embodiments, the contacting of the test sample with the capture antibody and the detection antibody is carried out sequentially, in either order.

In some of any of the provided embodiments, the test sample is contacted with the capture antibody prior to being contacted with the detection antibody, wherein the test sample is contacted with the capture antibody under conditions to form a first complex comprising the capture antibody and the one or more PAPP-A proteoforms; and the first complex is contacted with the detection antibody under conditions to form a second complex comprising the capture antibody, the one or more PAPP-A proteoforms, and the detection antibody.

Also provided herein in some embodiments is a method of detecting PAPP-A in a biological sample from a pregnant subject, comprising: (a) preparing a test sample from a pregnant female subject, the preparing comprising: (i) obtaining a biological sample from the pregnant female subject, wherein the obtained biological sample is a whole blood, serum, or plasma sample and wherein the obtained biological sample has a volume between or between about 0.5 μL and 10 μL, inclusive; and (ii) diluting the volume of the obtained biological sample in a sample diluent between or between about 2-fold and 100-fold, inclusive, thereby preparing the test sample; and (b) detecting in the test sample one or more PAPP-A proteoforms using an immunoassay, said one or more PAPP-A proteoforms comprising homodimeric PAPP-A and/or a heterotetrameric PAPP-A/proMBP complex, wherein the immunoassay comprises: (i) contacting the test sample with a capture antibody capable of specifically binding to (1) homodimeric PAPP-A and (2) a heterotetrameric PAPP-A/proMBP complex under conditions to form a first complex comprising the capture antibody and the one or more PAPP-A proteoforms; (ii) contacting the first complex comprising the capture antibody and the one or more PAPP-A proteoforms with a detection antibody capable of specifically binding to (1) homodimeric PAPP-A and (2) the heterotetrameric PAPP-A/proMBP complex under conditions to form a second complex comprising the capture antibody, the one or more PAPP-A proteoforms, and the detection antibody, wherein the detection antibody is conjugated to a detectable label that is capable of producing a detectable signal; and (iii) assessing from the test sample the degree of the detectable signal produced by the detectable label.

In some of any of the provided embodiments, the test sample is contacted with the detection antibody prior to being contacted with the capture antibody, wherein the test sample is contacted with the detection antibody under conditions to form a first complex comprising the detection antibody and the one or more PAPP-A proteoforms; and the first complex is contacted with the capture antibody under conditions to form a second complex comprising the capture antibody, the one or more PAPP-A proteoforms, and the detection antibody.

Also provided herein in some embodiments is a method of detecting PAPP-A in a biological sample from a pregnant subject, comprising: (a) preparing a test sample from a pregnant female subject, the preparing comprising: (i) obtaining a biological sample from the pregnant female subject, wherein the obtained biological sample is a whole blood, serum, or plasma sample and wherein the obtained biological sample has a volume between or between about 0.5 μL and 10 μL, inclusive; and (ii) diluting the volume of the obtained biological sample in a sample diluent between or between about 2-fold and 100-fold, inclusive, thereby preparing the test sample; and (b) detecting in the test sample one or more PAPP-A proteoforms using an immunoassay, said one or more PAPP-A proteoforms comprising homodimeric PAPP-A and/or a heterotetrameric PAPP-A/proMBP complex, wherein the immunoassay comprises: (i) contacting the test sample with a detection antibody capable of specifically binding to (1) homodimeric PAPP-A and (2) a heterotetrameric PAPP-A/proMBP complex under conditions to form a first complex comprising the detection antibody and the one or more PAPP-A proteoforms, wherein the detection antibody is conjugated to a detectable label that is capable of producing a detectable signal; (ii) contacting the first complex comprising the detection antibody and the one or more PAPP-A proteoforms with a capture antibody capable of specifically binding to (1) homodimeric PAPP-A and (2) the heterotetrameric PAPP-A/proMBP complex under conditions to form a second complex comprising the capture antibody, the one or more PAPP-A proteoforms, and the detection antibody; and (iii) assessing from the test sample the degree of the detectable signal produced by the detectable label.

In some of any of the provided embodiments, when the biological sample is obtained, the gestational age (GA) of the pregnant female subject is suspected to be between or between about 5 weeks and 40 weeks, between or between about 5 weeks and 30 weeks, or between or between about 5 weeks and 20 weeks, or between or between about 5 weeks and 15 weeks, each inclusive. In some of any of the provided embodiments, when the biological sample is obtained, the gestational age (GA) of the pregnant female subject is suspected to be between or between about 5 weeks and 20 weeks, inclusive. In some of any of the provided embodiments, when the biological sample is obtained, the gestational age (GA) of the pregnant female subject is suspected to be between or between about 5 weeks and 15 weeks, inclusive. In some of any of the provided embodiments, when the biological sample is obtained, the gestational age (GA) of the pregnant female subject is suspected to be between or between about 5 weeks and 10 weeks, inclusive.

In some embodiments, when the biological sample is obtained, the GA of the pregnant female subject is suspected to be between or between about 35 days and 70 days, inclusive. In some embodiments, when the biological sample is obtained, the GA of the pregnant female subject is suspected to be between or between about 35 days and 69 days, inclusive. In some embodiments, when the biological sample is obtained, the GA of the pregnant female subject is suspected to be between or between about 35 days and 68 days, inclusive. In some embodiments, when the biological sample is obtained, the GA of the pregnant female subject is suspected to be between or between about 35 days and 67 days, inclusive. In some embodiments, when the biological sample is obtained, the GA of the pregnant female subject is suspected to be between or between about 35 days and 66 days, inclusive. In some embodiments, when the biological sample is obtained, the GA of the pregnant female subject is suspected to be between or between about 35 days and 65 days, inclusive. In some embodiments, when the biological sample is obtained, the GA of the pregnant female subject is suspected to be between or between about 35 days and 64 days, inclusive. In some embodiments, when the biological sample is obtained, the GA of the pregnant female subject is suspected to be between or between about 35 days and 63 days, inclusive.

In some of any of the provided embodiments, the immunoassay is a colorimetric assay.

In some of any of the provided embodiments, the immunoassay is a solid-phase immunoassay.

In some of any of the provided embodiments, the immunoassay is an Enzyme linked immunosorbent assay (ELISA). In some of any of the provided embodiments, the immunoassay is a sandwich ELISA.

In some of any of the provided embodiments, the immunoassay is a lateral flow assay.

In some of any of the provided embodiments, the method further comprises classifying the GA of the pregnant female subject based on the degree of the detectable signal assessed from the test sample.

In some of any of the provided embodiments, the method further comprises determining the concentration of PAPP-A in the obtained biological sample by comparison of the degree of the detectable signal measured from the test sample to a standard curve.

Also provided herein in some embodiments is a method of measuring the concentration of PAPP-A in a biological sample from a pregnant subject, comprising: (a) preparing a test sample from a pregnant female subject, the preparing comprising: (i) obtaining a biological sample from the pregnant female subject, wherein the obtained biological sample is a whole blood, serum, or plasma sample and wherein the obtained biological sample has a volume between or between about 0.5 μL and 10 μL, inclusive; and (ii) diluting the volume of the obtained biological sample in a sample diluent between or between about 2-fold and 100-fold, inclusive, thereby preparing the test sample; and (b) determining the concentration of PAPP-A in the obtained biological sample using an immunoassay, wherein the immunoassay comprises detecting one or more PAPP-A proteoforms in the test sample, said one or more PAPP-A proteoforms comprising homodimeric PAPP-A and/or a heterotetrameric PAPP-A/proMBP complex, said detecting comprising: (i) contacting the test sample with a capture antibody capable of specifically binding to (1) homodimeric PAPP-A and (2) a heterotetrameric PAPP-A/proMBP complex under conditions to form a first complex comprising the capture antibody and the one or more PAPP-A proteoforms; (ii) contacting the first complex comprising the capture antibody and the one or more PAPP-A proteoforms with a detection antibody capable of specifically binding to (1) homodimeric PAPP-A and (2) the heterotetrameric PAPP-A/proMBP complex under conditions to form a second complex comprising the capture antibody, the one or more PAPP-A proteoforms, and the detection antibody, wherein the detection antibody is conjugated to a detectable label that is capable of producing a detectable signal; and (iii) assessing the degree of the detectable signal produced by the detectable label; and (iv) determining the concentration of PAPP-A in the obtained biological sample by comparison of the degree of the detectable signal from the test sample to a standard curve.

Also provided herein in some embodiments is a method of measuring the concentration of PAPP-A in a biological sample from a pregnant subject, comprising: (a) preparing a test sample from a pregnant female subject, the preparing comprising: (i) obtaining a biological sample from the pregnant female subject, wherein the obtained biological sample is a whole blood, serum, or plasma sample and wherein the obtained biological sample has a volume between or between about 0.5 μL and 10 μL inclusive; and (ii) diluting the volume of the obtained biological sample in a sample diluent between or between about 2-fold and 100-fold, inclusive, thereby preparing the test sample; and (b) determining the concentration of PAPP-A in the obtained biological sample using an immunoassay, wherein the immunoassay comprises detecting one or more PAPP-A proteoforms in the test sample, said one or more PAPP-A proteoforms comprising homodimeric PAPP-A and/or a heterotetrameric PAPP-A/proMBP complex, said detecting comprising: (i) contacting the test sample with a detection antibody capable of specifically binding to (1) homodimeric PAPP-A and (2) a heterotetrameric PAPP-A/proMBP complex under conditions to form a first complex comprising the detection antibody and the one or more PAPP-A proteoforms, wherein the detection antibody is conjugated to a detectable label that is capable of producing a detectable signal; (ii) contacting the first complex comprising the detection antibody and the one or more PAPP-A proteoforms with a capture antibody capable of specifically binding to (1) homodimeric PAPP-A and (2) the heterotetrameric PAPP-A/proMBP complex under conditions to form a second complex comprising the capture antibody, the one or more PAPP-A proteoforms, and the detection antibody; (iii) assessing the degree of the detectable signal produced by the detectable label; and (iv) determining the concentration of PAPP-A in the obtained biological sample by comparison of the degree of the detectable signal from the test sample to a standard curve.

In some of any of the provided embodiments, the method further comprises classifying the GA of the pregnant female subject based on the concentration of PAPP-A in the obtained biological sample.

Also provided herein in some embodiments is a method for classifying the gestational age (GA) of a pregnancy, comprising: (a) detecting PAPP-A in a biological sample obtained from a pregnant female subject according to the method of some of any of the provided embodiments; (b) comparing the degree of the detectable signal assessed from the test sample to the degree of a detectable signal assessed using the immunoassay from a reference PAPP-A sample, wherein the reference PAPP-1 sample comprises (1) homodimeric PAPP-A and/or (2) the heterotetrameric PAPP-A/proMBP complex, and wherein the concentration of the reference PAPP-A sample is a predetermined concentration associated with a predetermined GA cutpoint using the immunoassay; and (c) classifying: the GA of the pregnancy as less than the predetermined GA cutpoint if the degree of the detectable signal assessed from the test sample is lower than the degree of the detectable signal assessed from the reference sample; or the GA of the pregnancy as greater than or equal to the predetermined GA cutpoint if the degree of the detectable signal assessed from the test sample is higher than or equal to the degree of the detectable signal assessed from the reference sample.

In some embodiments, the reference PAPP-A sample comprises the heterotetrameric PAPP-A/proMBP complex.

Also provided herein in some embodiments is a method for classifying the gestational age (GA) of a pregnancy, comprising: (a) detecting PAPP-A in a biological sample obtained from a pregnant female subject using an immunoassay, wherein: the biological sample is a whole blood, serum, or plasma sample; and the immunoassay comprises detecting one or more PAPP-A proteoforms in a test sample derived from the biological sample, said one or more PAPP-A proteoforms comprising homodimeric PAPP-A or a heterotetrameric PAPP-A/proMBP complex and said detecting comprising: (i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and (ii) assessing the degree of the detectable signal produced by the detectable label; (b) comparing the degree of the detectable signal assessed from the test sample to the degree of a detectable signal measured using the immunoassay from a reference PAPP-A sample, wherein the reference PAPP-A sample comprises the heterotetrameric PAPP-A/proMBP complex, and wherein the concentration of the reference PAPP-A sample is a predetermined concentration associated with a predetermined GA cutpoint; and (c) classifying: the GA of the pregnancy as less than the predetermined GA cutpoint if the degree of the detectable signal assessed from the test sample is lower than the degree of the detectable signal assessed from the reference sample; or the GA of the pregnancy as greater than or equal to the predetermined GA cutpoint if the degree of the detectable signal assessed from the test sample is higher than or equal to the degree of the detectable signal assessed from the reference sample.

In some of any of the provided embodiments, the reference PAPP-A sample further comprises homodimeric PAPP-A. In some of any of the provided embodiments, the reference PAPP-A sample consists essentially of the heterotetrameric PAPP-A/proMBP complex.

Also provided herein in some embodiments is a method for classifying the gestational age (GA) of a pregnancy, comprising: (a) determining the concentration of PAPP-A in a biological sample obtained from a pregnant female subject according to the method of some of any of the provided embodiments; (b) comparing the concentration of PAPP-A in the obtained biological sample to a predetermined concentration of PAPP-A, wherein the predetermined concentration is associated with a predetermined GA cutpoint using the immunoassay; and (c) classifying: the GA of the pregnancy as less than a predetermined GA cutpoint if the concentration of PAPP-A in the obtained biological sample is lower than the predetermined concentration; or the GA of the pregnancy as greater than or equal to the predetermined GA cutpoint if the concentration of PAPP-A in the obtained biological sample is higher than or equal to the predetermine concentration.

In some of any of the provided embodiments, the predetermined GA cutpoint is a timepoint between or between about 5 weeks and 40 weeks, between or between about 5 weeks and 30 weeks, or between or between about 5 weeks and 20 weeks, each inclusive. In some of any of the provided embodiments, the predetermined GA cutpoint is a timepoint between or between about 5 weeks and 15 weeks, inclusive.

In some of any of the provided embodiments, the predetermined GA cutpoint is a timepoint between or between about 60 days and 140 days, inclusive. In some of any of the provided embodiments, the predetermined GA cutpoint is or is about 70 days.

In some of any of the provided embodiments, the predetermined concentration of PAPP-A is between or between about 20 ng/mL and 200 ng/mL, inclusive. In some of any of the provided embodiments, the predetermined concentration of PAPP-A is between or between 30 ng/mL and 150 ng/mL, inclusive.

In some of any of the provided embodiments, the predetermined concentration is between or between about 150 ng/mL and 250 ng/mL, inclusive. In some of any of the provided embodiments, the predetermined concentration is between or between 160 ng/mL and 200 ng/mL, inclusive. In some of any of the provided embodiments, the predetermined concentration is or is about 180.679 ng/mL. In some embodiments, the predetermined concentration is for a predetermined GA that is 70 days+/−2 weeks. In some embodiments, the predetermined concentration is for a predetermined GA that is at or about 63 days. In some embodiments, the predetermined concentration is for a predetermined GA that is at or about 70 days. In some embodiments, the predetermined concentration is for a predetermined GA that is at or about 77 days.

Also provided herein in some embodiments is method for classifying the gestational age (GA) of a pregnancy, comprising: (a) detecting PAPP-A in a biological sample obtained from a pregnant female subject according to the method of some of any of the provided embodiments; (b) detecting PAPP-A in a first reference PAPP-A sample using the immunoassay, thereby assessing the degree of a detectable signal from the first reference PAPP-A sample, wherein the first reference PAPP-A sample comprises (1) homodimeric PAPP-A and/or (2) the heterotetrameric PAPP-A/proMBP complex and wherein the concentration of the first reference PAPP-A sample is a first predetermined concentration associated with a first predetermined GA cutpoint using the immunoassay; (c) detecting PAPP-A in a second reference PAPP-A sample using the immunoassay, thereby assessing the degree of a detectable signal from the second reference PAPP-A sample, wherein the second reference PAPP-A sample comprises (1) homodimeric PAPP-A and/or (2) the heterotetrameric PAPP-A/proMBP complex and wherein the concentration of the second reference PAPP-A sample is a second predetermined concentration associated with a second predetermined GA cutpoint that is later than the first predetermined GA cutpoint using the immunoassay; (d) comparing the degree of the detectable signal assessed from the test sample to the degree of the detectable signal assessed from the first reference PAPP-A sample and to the degree of the detectable signal assessed from the second reference PAPP-A sample; and (e) classifying: the GA of the pregnancy as less than the first predetermined GA cutpoint if the degree of the detectable signal assessed from the test sample is lower than the degree of the detectable signal assessed from the first reference PAPP-A sample; the GA of the pregnancy as between the first and second predetermined GA cutpoints if the degree of the detectable signal assessed from the test sample is higher than the degree of the detectable signal assessed from the first reference PAPP-A sample and lower than the degree of the detectable signal measured from the second reference PAPP-A sample; or the GA of the pregnancy as greater than or equal to the second predetermined GA cutpoint if the degree of the detectable signal assessed from the test sample is higher than the degree of the detectable signal assessed from the second reference PAPP-A sample.

Also provided herein in some embodiments is a method for classifying the gestational age (GA) of a pregnancy, comprising: (a) detecting PAPP-A in a biological sample obtained from a pregnant female subject using an immunoassay, wherein: the biological sample is a whole blood, serum, or plasma sample; and the immunoassay comprises detecting one or more PAPP-A proteoforms in a test sample derived from the biological sample, said one or more PAPP-A proteoforms comprising homodimeric PAPP-A or a heterotetrameric PAPP-A/proMBP complex and said detecting comprising: (i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and (ii) assessing the degree of the detectable signal produced by the detectable label; (b) detecting PAPP-A in a first reference PAPP-A sample using the immunoassay, thereby measuring the degree of a detectable signal from the first reference PAPP-A sample, wherein the first reference PAPP-A sample comprises (1) homodimeric PAPP-A and/or (2) the heterotetrameric PAPP-A/proMBP complex and wherein the concentration of the first reference PAPP-A sample is a predetermined concentration associated with a first predetermined GA cutpoint using the immunoassay; (c) detecting PAPP-A in a second reference PAPP-A sample using the immunoassay, thereby measuring the degree of a detectable signal from the second reference PAPP-A sample, wherein the second reference PAPP-A sample comprises (1) homodimeric PAPP-A and/or (2) the heterotetrameric PAPP-A/proMBP complex and wherein the concentration of the second reference PAPP-A sample is a second predetermined concentration associated with a second predetermined GA cutpoint that is later than the first predetermined GA cutpoint; (d) comparing the degree of the detectable signal assessed from the test sample to the degree of the detectable signal assessed from the first reference PAPP-A sample and to the degree of the detectable signal assessed from the second reference PAPP-A sample; and (e) classifying: the GA of the pregnancy as less than the first predetermined GA cutpoint if the degree of the detectable signal assessed from the test sample is lower than the degree of the detectable signal assessed from the first reference PAPP-A sample; the GA of the pregnancy as between the first and second predetermined GA cutpoints if the degree of the detectable signal assessed from the test sample is higher than the degree of the detectable signal assessed from the first reference PAPP-A sample and lower than the degree of the detectable signal assessed from the second reference PAPP-A sample; or the GA of the pregnancy as greater than or equal to the second predetermined GA cutpoint if the degree of the detectable signal assessed from the test sample is higher than the degree of the detectable signal assess from the second reference PAPP-A sample.

In some of any provided embodiments, the contacting of the test sample with the capture antibody and the detection antibody is carried out simultaneously.

In some of any of the provided embodiments, the contacting of the test sample with the capture antibody and the detection antibody is carried out sequentially in either order. In some embodiments, the test sample is contacted with the capture antibody prior to being contacted with the detection antibody, wherein the test sample is contacted with the capture antibody under conditions to form a first complex comprising the capture antibody and the one or more PAPP-A proteoforms; and the first complex is contacted with the detection antibody under conditions to form a second complex comprising the capture antibody, the one or more PAPP-A proteoforms, and the detection antibody. In some embodiments, the test sample is contacted with the detection antibody prior to being contacted with the capture antibody, wherein the test sample is contacted with the detection antibody under conditions to form a first complex comprising the detection antibody and the one or more PAPP-A proteoforms; and the first complex is contacted with the capture antibody under conditions to form a second complex comprising the capture antibody, the one or more PAPP-A proteoforms, and the detection antibody.

In some of any of the provided embodiments, when the biological sample is obtained, the gestational age (GA) of the pregnant female subject is suspected to be between or between about 5 weeks and 40 weeks, between or between about 5 weeks and 30 weeks, or between or between about 5 weeks and 20 weeks, each inclusive. In some embodiments, when the biological sample is obtained, the GA of the pregnant female subject is suspected to be between or between about 5 weeks and 15 weeks, inclusive. In some embodiments, when the biological sample is obtained, the GA of the pregnant female subject is suspected to be between or between about 5 weeks and 10 weeks, inclusive.

In some embodiments, when the biological sample is obtained, the GA of the pregnant female subject is suspected to be between or between about 35 days and 70 days, inclusive. In some embodiments, when the biological sample is obtained, the GA of the pregnant female subject is suspected to be between or between about 35 days and 69 days, inclusive. In some embodiments, when the biological sample is obtained, the GA of the pregnant female subject is suspected to be between or between about 35 days and 68 days, inclusive. In some embodiments, when the biological sample is obtained, the GA of the pregnant female subject is suspected to be between or between about 35 days and 67 days, inclusive. In some embodiments, when the biological sample is obtained, the GA of the pregnant female subject is suspected to be between or between about 35 days and 66 days, inclusive. In some embodiments, when the biological sample is obtained, the GA of the pregnant female subject is suspected to be between or between about 35 days and 65 days, inclusive. In some embodiments, when the biological sample is obtained, the GA of the pregnant female subject is suspected to be between or between about 35 days and 64 days, inclusive. In some embodiments, when the biological sample is obtained, the GA of the pregnant female subject is suspected to be between or between about 35 days and 63 days, inclusive.

In some of any of the provided embodiments, the immunoassay is a colorimetric assay. In some of any provided embodiments, the immunoassay is a solid-phase immunoassay. In some of any provided embodiments, the immunoassay is an Enzyme linked immunosorbent assay (ELISA), optionally a sandwich ELISA. In some of any provided embodiments, the immunoassay is a lateral flow assay.

In some of any provided embodiments, the first and second predetermined GA cutpoints are timepoints between or between about 5 weeks and 40 weeks, between or between about 5 weeks and 30 weeks, or between or between about 5 weeks and 20 weeks, each inclusive.

In some of any of the provided embodiments, the first and second predetermined GA cutpoints are timepoints between or between about 5 weeks and 15 weeks, inclusive.

In some of any of the provided embodiments, the first and second predetermined GA cutpoints are timepoints between or between about 60 days and 140 days, inclusive.

In some of any of the provided embodiments, the first predetermined GA cutpoint is or is about 70 days+/−2 weeks, and the second predetermined GA cutpoint is or is about 105 days+/−1 week.

In some of any of the provided embodiments, the first predetermined GA cutpoint is or is about 63 days, and the second predetermined GA cutpoint is or is about 105 days+/−1 week. In some of any of the provided embodiments, the first predetermined GA cutpoint is or is about 70 days, and the second predetermined GA cutpoint is or is about 105 days+/−1 week. In some embodiments, the first predetermined GA cutpoint is or is about 77 days, and the second predetermined GA cutpoint is or is about 105 days+/−1 week. In some embodiments, the second predetermined GA cutpoint is at or about 98 days. In some embodiments, the second predetermined GA cutpoint is at or about 112 days.

In some of any of the provided embodiments, the classifying is performed with greater than or greater than about 80% sensitivity and/or greater than or greater than about 80% specificity. In some of any of the provided embodiments, the classifying is performed with greater than or greater than about 85% sensitivity and/or greater than or greater than about 85% specificity. In some of any of the provided embodiments, the classifying is performed with greater than or greater than about 90% sensitivity and/or greater than or greater than about 90% specificity.

In some of any of the provided embodiments, the classifying is further based on whether the subject was using tobacco products at the time the biological sample was obtained. In some embodiments, the methods thus may further include determining if the subject uses tobacco products. In some of any of the provided embodiments, the GA of the pregnancy is more likely to be classified as greater than or equal to the predetermined GA cutpoint if the subject was using tobacco products.

In some of any of the provided embodiments, the classifying is further based on the body mass index (BMI) of the subject at the time the biological sample was obtained. In some embodiments, the methods further include determining the BMI of the subject. In some of any of the provided embodiments, the GA of the pregnancy is more likely to be classified as greater than or equal to the predetermined GA cutpoint if the BMI of the subject is high, such as BMI indicative of a subject that is overweight (e.g., BMI of 25 to 29.9) or, in some cases, obese (e.g., BMI of 30 and above). In some embodiments, a high BMI is a BMI that is at or greater than 25.

Provided herein is a method for screening a pregnant subject for a prenatal care or prenatal clinical treatment, comprising (a) classifying the gestational age (GA) of a pregnancy according to any of the provided embodiments for classifying GA; and (b) based on the classifying, (i) selecting the pregnant female subject as eligible for a prenatal care or prenatal clinical treatment if the GA of the pregnancy is classified as less than the predetermined GA cutpoint; or (ii) selecting the pregnant female subject as not eligible for the prenatal care or prenatal clinical treatment or as a candidate for further assessment for the prenatal care or prenatal clinical treatment if the GA of the pregnancy is classified as greater than or equal to the predetermined GA cutpoint. Provided herein is a method for performing a prenatal care or prenatal clinical treatment on a pregnant subject, comprising performing a prenatal care or prenatal clinical treatment on a pregnant female subject selected as eligible for the prenatal care or prenatal clinical treatment according to the preceding method.

Provided herein is a method for determining the gestational age (GA) of a pregnancy, comprising: (a) detecting PAPP-A in a biological sample obtained from a pregnant female subject according to any of the provided methods for detecting PAPP-A; and (b) determining the GA of the pregnancy based on the degree of the detectable signal assessed from the test sample, wherein the determining comprises providing the degree of the detectable signal assessed from the test sample as input to a process that uses the degree of the detectable signal measured from the test sample to predict GA.

Provided herein is a method for determining the gestational age (GA) of a pregnancy, comprising (a) detecting PAPP-A in a biological sample obtained from a pregnant female subject using an immunoassay, wherein: the biological sample is a whole blood, serum, or plasma sample; and the immunoassay comprises detecting one or more PAPP-A proteoforms in a test sample derived from the biological sample, said one or more PAPP-A proteoforms comprising homodimeric PAPP-A or a heterotetrameric PAPP-A/proMBP complex, said detecting comprising: (i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and (ii) measuring the degree of the detectable signal produced by the detectable label; and (b) determining the GA of the pregnancy based on the degree of the detectable signal assessed from the test sample, wherein the determining comprises providing the degree of the detectable signal assessed from the test sample as input to a process that uses the degree of the detectable signal assessed from the test sample to predict GA.

Provided herein is a method for determining the gestational age (GA) of a pregnancy, comprising: (a) measuring the concentration of PAPP-A in a biological sample obtained from a pregnant female subject according to any of the provided methods for determining a concentration; and (b) determining the GA of the pregnancy based on the concentration of PAPP-A in the obtained biological sample, wherein the determining comprises providing the concentration of PAPP-A in the obtained biological sample as input to a process that uses PAPP-A concentration as a continuous predictor of GA.

Provided herein is a method for determining the gestational age (GA) of a pregnancy, comprising: (a) determining the concentration of PAPP-A in a biological sample obtained from a pregnant female subject using an immunoassay, wherein: the biological sample is a whole blood, serum, or plasma sample; and the immunoassay comprises detecting one or more PAPP-A proteoforms in a test sample derived from the biological sample, said one or more PAPP-A proteoforms comprising homodimeric PAPP-A or a heterotetrameric PAPP-A/proMBP complex, said detecting comprising: (i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and (ii) assessing the degree of the detectable signal produced by the detectable label; and (iii) determining the concentration of PAPP-A in the obtained biological sample by comparison of the degree of the detectable signal to a standard curve; and (b) determining the GA of the pregnancy based on the concentration of PAPP-A in the obtained biological sample, wherein the determining comprises providing the concentration of PAPP-A in the obtained biological sample as input to a process that uses PAPP-A concentration as a continuous predictor of GAs.

In some of any of the provided embodiments, the contacting of the test sample with the capture antibody and the detection antibody is carried out simultaneously.

In some of any of the provided embodiments, the contacting of the test sample with the capture antibody and the detection antibody is carried out sequentially in either order. In some embodiments, the test sample is contacted with the capture antibody prior to being contacted with the detection antibody, wherein: the test sample is contacted with the capture antibody under conditions to form a first complex comprising the capture antibody and the one or more PAPP-A proteoforms; and the first complex is contacted with the detection antibody under conditions to form a second complex comprising the capture antibody, the one or more PAPP-A proteoforms, and the detection antibody. In some embodiments, the test sample is contacted with the detection antibody prior to being contacted with the capture antibody, wherein: the test sample is contacted with the detection antibody under conditions to form a first complex comprising the detection antibody and the one or more PAPP-A proteoforms; and the first complex is contacted with the capture antibody under conditions to form a second complex comprising the capture antibody, the one or more PAPP-A proteoforms, and the detection antibody.

In some of any of the provided embodiments, when the biological sample is obtained, the gestational age (GA) of the pregnant female subject is suspected to be between or between about 5 weeks and 40 weeks, between or between about 5 weeks and 30 weeks, or between or between about 5 weeks and 20 weeks, each inclusive. In some of any of the provided embodiments, when the biological sample is obtained, the GA of the pregnant female subject is suspected to be between or between about 5 weeks and 15 weeks, inclusive. In some of any of the provided embodiments, when the biological sample is obtained, the GA of the pregnant female subject is suspected to be between or between about 5 weeks and 10 weeks, inclusive.

In some embodiments, when the biological sample is obtained, the GA of the pregnant female subject is suspected to be between or between about 35 days and 70 days, inclusive. In some embodiments, when the biological sample is obtained, the GA of the pregnant female subject is suspected to be between or between about 35 days and 69 days, inclusive. In some embodiments, when the biological sample is obtained, the GA of the pregnant female subject is suspected to be between or between about 35 days and 68 days, inclusive. In some embodiments, when the biological sample is obtained, the GA of the pregnant female subject is suspected to be between or between about 35 days and 67 days, inclusive. In some embodiments, when the biological sample is obtained, the GA of the pregnant female subject is suspected to be between or between about 35 days and 66 days, inclusive. In some embodiments, when the biological sample is obtained, the GA of the pregnant female subject is suspected to be between or between about 35 days and 65 days, inclusive. In some embodiments, when the biological sample is obtained, the GA of the pregnant female subject is suspected to be between or between about 35 days and 64 days, inclusive. In some embodiments, when the biological sample is obtained, the GA of the pregnant female subject is suspected to be between or between about 35 days and 63 days, inclusive.

In some of any of the provided embodiments, the immunoassay is a colorimetric assay. In some of any of the provided embodiments, the immunoassay is a solid-phase immunoassay. In some of any of the provided embodiments, the immunoassay is an Enzyme linked immunosorbent assay (ELISA), optionally a sandwich ELISA. In some of any of the provided embodiments, the immunoassay is a lateral flow assay.

In some of any of the provided embodiments for determining GA, the process comprises a regression model trained using GAs and PAPP-A concentrations from a plurality of pregnant female subjects. In some embodiments, the regression model is a linear regression model, a piecewise linear model, a polynomial regression model, or a Bayesian model.

In some of any of the provided embodiments for determining the GA age, the process predicts GAs between or between about 5 weeks and 40 weeks, between or between about 5 weeks and 30 weeks, or between or between about 5 weeks and 20 weeks, each inclusive. In some of any embodiments of determining the GA, the process predicts GAs between or between about 5 weeks and 15 weeks, inclusive. In some of any embodiments of determining the GA age, the process predicts GAs between or between about 10 weeks and 15 weeks, inclusive.

In some of any of the provided embodiments, the determining further comprises providing whether or not the subject was using tobacco products at the time the biological sample was obtained as input to the process. In some of any of the provided embodiments, the process further uses whether the subject was using tobacco products as a predictor of GA. In some of any of the provided embodiments, the regression model is trained further using whether or not each of the plurality of pregnant female subjects was using tobacco products, e.g., when a biological sample was obtained from the pregnant female subject.

In some of any of the provided embodiments, the determining further comprises providing the body mass index (BMI) of the subject at the time the biological sample was obtained as input to the process. In some of any of the provided embodiments, the process further uses the BMI as a predictor of GA. In some of any of the provided embodiments, the regression model is trained further using the BMI of each of the plurality of pregnant female subjects, e.g., when a biological sample was obtained from the pregnant female subject.

Provided herein is a method of selecting a prenatal care or prenatal clinical treatment for a pregnant subject, comprising: determining the gestational age (GA) of a pregnancy according to the any of the provided methods and (b) selecting a prenatal care or prenatal clinical treatment for the pregnant female subject based on the GA of the pregnancy. Provided herein is a method for performing a prenatal care or prenatal clinical treatment on a pregnant subject, comprising performing a prenatal care or prenatal clinical treatment on a pregnant female subject, wherein the prenatal care or prenatal clinical treatment is selected according to the preceding method.

In some of any of the provided embodiments, the prenatal care or prenatal clinical treatment is a medical abortion or early aspiration; a decision about a medical abortion regimen; a decision about the risk of embryotoxicity; a clinical examination; a vaccination; a risk assessment; a fetal assessment; a blood assay; a urine assay; vitamin supplementation; a test for disease; education; counseling; or any combination of any of the foregoing. In some embodiments, the prenatal care or prenatal clinical treatment is a medical abortion or early aspiration.

In some of any of the provided embodiments, the obtained biological sample has a volume between or between about 0.5 μL and 10 μL, inclusive. In some of any of the provided embodiments, the obtained biological sample has a volume between or between about 0.5 μL and 5 μL, inclusive. In some embodiments, the obtained biological sample has a volume of or of about 1 μL.

In some of any of the provided embodiments, the test sample is prepared by diluting the obtained biological sample with a sample diluent prior to the measuring. In some of any of the provided embodiments, the method further comprises diluting the obtained biological sample with a sample diluent prior to the measuring. In some of any of the provided embodiments, the volume of the obtained biological sample is diluted between or between about 2-fold and 100-fold, inclusive. In some embodiments, the volume of the obtained biological sample is diluted between or between about 2-fold and 75-fold, inclusive. In some embodiments, the volume of the obtained biological sample is diluted or diluted about 5-fold or 50-fold.

In some of any of the provided embodiments, the sample diluent comprises one or both of buffered saline and a nonionic detergent. In some of any of the provided embodiments, the sample diluent does not comprise a metal chelator. In some of any of the provided embodiments, the sample diluent does not comprise EDTA.

In some of any of the provided embodiments, the obtained biological sample is a whole blood sample In some embodiments, the obtained biological sample is a serum sample. In some of any of the provided embodiments, the method further comprises obtaining the biological sample from the pregnant female subject.

In some of any of the provided embodiments, the capture antibody and/or the detection antibody is capable of specifically binding to (1) homodimeric PAPP-A and (2) the heterotetrameric PAPP-A/proMBP complex equimolarly or about equimolarly. In some of any of the provided embodiments, the capture antibody is unlabeled. In some of any of the provided embodiments, the capture antibody is immobilized on a solid support. In some of any of the provided embodiments, the solid support is a bead, column, array, assay plate, microwell, cartridge, stick, filter, or strip. In some embodiments, the solid support is formed of glass, polysaccharides, polyacrylamides, polystyrene, polyvinyl alcohol, nitrocellulose, cellulose, nylon, and/or silicones.

In some of any of the provided embodiments, the capture antibody and/or the detection antibody does not specifically bind to proMBP. In some of any of the provided embodiments, the capture antibody and/or the detection antibody does not cross-react with proMBP. In some of any of the provided embodiments, the capture antibody and/or the detection antibody does not cross-react with proMBP at up to twice the physiological concentrations of proMBP. In some of any of the provided embodiments, the capture antibody and/or the detection antibody does not specifically bind to PAPP-A2. In some of any of the provided embodiments, the capture antibody and/or the detection antibody does not cross-react with PAPP-A2. In some of any of the provided embodiments, the capture antibody and/or the detection antibody does not cross-react with PAPP-A2 at up to twice the physiological concentrations of PAPP-A2. In some of any of the provided embodiments, the capture antibody and/or the detection antibody does not specifically bind to MMP-9. In some of any of the provided embodiments, the capture antibody and/or the detection antibody does not cross-react with MMP-9. In some of any of the provided embodiments, the capture antibody and/or the detection antibody does not cross-react with MMP-9 at up to twice the physiological concentrations of MMP-9. In some of any of the provided embodiments, the physiological concentrations are with respect to serum concentration levels. In some of any of the provided embodiments, the physiological concentrations are with respect to serum concentration levels in pregnant subjects.

In some of any of the provided embodiments, the capture antibody and/or the detection antibody specifically binds to a PAPP-A subunit of homodimeric PAPP-A. In some of any of the provided embodiments, the capture antibody and/or the detection antibody specifically binds to a PAPP-A subunit of the heterotetrameric PAPP-A/proMBP complex. In some of any of the provided embodiments, the capture antibody and/or the detection antibody specifically binds to a PAPP-A subunit of homodimeric PAPP-A and specifically binds to a PAPP-A subunit of the heterotetrameric PAPP-A/proMBP complex.

In some of any of the provided embodiments, the capture antibody and the detection antibody specifically binds to a PAPP-A subunit of homodimeric PAPP-A. In some of any of the provided embodiments, the capture antibody and the detection antibody specifically binds to a PAPP-A subunit of the heterotetrameric PAPP-A/proMBP complex. In some of any of the provided embodiments, the capture antibody and the detection antibody specifically binds to a PAPP-A subunit of homodimeric PAPP-A and specifically binds to a PAPP-A subunit of the heterotetrameric PAPP-A/proMBP complex.

In some of any of the provided embodiments, the capture antibody and/or the detection antibody does not bind the metal chelating region of PAPP-A.

In some of any of the provided embodiments, the detectable label is or comprises horseradish peroxidase.

In some of any of the provided embodiments, the method is carried out using a point-of-care device. In some of any of the provided embodiments, the method is carried out using a lateral flow stick.

In some of any of the provided embodiments, the method is carried out in a laboratory. In some embodiments, the method is carried out without an ultrasound. In some embodiments, the method further comprises comparing the classified GA to a GA as determined by performing an ultrasound.

Provided herein is a device for carrying out any of the provided methods, such as methods including detecting PAPP-A or for determining the concentration of PAPP-A. In some embodiments, the devise is a hand-held device or a point-of-care device. In some embodiments, the device comprises a solid support and one or more antibody capable of independently specifically binding to (1) homodimeric PAPP-A and (2) a heterotetrameric PAPP-A/proMBP complex, wherein at least one of the one or more antibody is immobilized on the solid support. In some embodiments, the solid support is a bead, column, array, assay plate, microwell, cartridge, stick, filter, or strip. In some embodiments, the solid support is formed of glass, polysaccharides, polyacrylamides, polystyrene, polyvinyl alcohol, nitrocellulose, cellulose, nylon, and/or silicones. In some embodiments, the device is a lateral flow stick. In some embodiments, the device carries out a lateral flow immunoassay.

Provided herein is a kit for carrying out any of the provided methods comprising a reference PAPP-A sample and any of the provided devices, wherein: the concentration of PAPP-A in the reference PAPP-A sample is for a predetermined GA cutpoint; and the PAPP-A in the reference PAPP-A sample comprises heterotetrameric PAPP-A/proMBP complex. In some embodiments, the PAPP-A in the reference PAPP-A sample further comprises homodimeric PAPP-A. In some embodiments, the PAPP-A in the reference PAPP-A sample consists essentially of heterotetrameric PAPP-A/proMBP complex.

In some embodiments of the provided kits, the predetermined GA cutpoint is a timepoint between or between about 5 weeks and 15 weeks, inclusive. In some embodiments, the predetermined GA cutpoint is a timepoint between or between about 64 days and 140 days, inclusive. In some embodiments, the predetermined GA cutpoint is or is about 63 days, 70 days, or 77 days. In some embodiments, the concentration of reference PAPP-A in the reference PAPP-A sample is between or between about 150 ng/mL and 250 ng/mL, inclusive. In some embodiments, the concentration of reference PAPP-A in the reference PAPP-A sample is between or between about 160 ng/mL and 200 ng/mL, inclusive. In some embodiments, the concentration of reference PAPP-A in the reference PAPP-A sample is or is about 180.679 ng/mL.

In some of any of the provided embodiments, the concentration of reference PAPP-A in the reference PAPP-A sample is between or between about 20 ng/mL and 200 ng/mL, inclusive. In some of any of the provided embodiments, the concentration of reference PAPP-A in the reference PAPP-A sample is between or between 30 ng/mL and 150 ng/mL, inclusive. In some embodiments, the concentration of reference PAPP-A in the reference PAPP-A sample is or is about 133.2096 ng/mL.

In some embodiments of any of the provided kits, the reference PAPP-A sample is a first reference PAPP-A sample and the predetermined GA cutpoint is a first predetermined GA cutpoint, and further comprising a second reference PAPP-A sample, wherein: the concentration of PAPP-A in the second reference PAPP-A sample is for a second predetermined GA cutpoint that is later than the first predetermined GA cutpoint; and the PAPP-A in the second reference sample comprises (1) homodimeric PAPP-A and/or (2) the heterotetrameric PAPP-A/proMBP complex. In some embodiments, the second predetermined GA cutpoint is a timepoint between or between about 5 weeks and 15 weeks, inclusive. In some embodiments, the second predetermined GA cutpoint is a timepoint between or between about 64 days and 140 days, inclusive. In some embodiments, the second predetermined GA cutpoint is or is about 63 days, 70 days, or 77 days. In some embodiments, the second predetermined GA cutpoint is or is about 105 days. In some embodiments, the first predetermined GA cutpoint is or is about 63 days, and the second predetermined GA cutpoint is or is about 105 days+/−1 week. In some embodiments, the first predetermined GA cutpoint is or is about 70 days, and the second predetermined GA cutpoint is or is about 105 days+/−1 week. In some embodiments, the first predetermined GA cutpoint is or is about 77 days, and the second predetermined GA cutpoint is or is about 105 days+/−1 week. In some embodiments, the second predetermined GA cutpoint is at or about 98 days. In some embodiments, the second predetermined GA cutpoint is at or about 112 days.

### BRIEF DESCRIPTION OF THE DRAWINGS

FIG. 1A shows receiver operating characteristic (ROC) plots for predicting gestational age (GA)>70 days using serum PAPP-A concentration measured with four different immunoassays (1-R&D Systems, 2-ThermoFisher, 3-Creative Diagnosis, and 4-Ansh Labs). FIG. 1B shows likelihood ratio (LR) graphs comparing the PAPP-A concentrations dichotomized by the optimal cut-off point for each assay.

FIGS. 2A-2D show serum PAPP-A concentration (y-axes) measured with four different immunoassays: 1-R&D Systems (FIG. 2A), 2-ThermoFisher (FIG. 2B), 3-Creative Diagnosis (FIG. 2C), and 4-Ansh Labs (FIG. 2D). Serum PAPP-A concentrations are plotted against GA (x-axes), with both variables expressed in base 10 logarithmic format. On each graph, the horizontal interrupted black line marks the respective assay's optimal cut-off point, which is also noted to the right of the line. The vertical interrupted black line marks the GA cut-off of 70 days (10 weeks). The bracket marks the respective assay's standard curve range.

FIGS. 3A-3D show serum PAPP-A concentration (y-axes) measured with four different immunoassays: 1-R&D Systems (FIG. 3A), 2-ThermoFisher (FIG. 3B), 3-Creative Diagnosis (FIG. 3C), and 4-Ansh Labs (FIG. 3D). Serum PAPP-A concentrations are plotted against GA (x-axes), with both variables expressed in base 10 logarithmic format. The fitted equations are also displayed. On each graph, the solid black line represents the regression line, the solid grey lines represent the 95% confidence interval, and the grey dotted lines represent the 95% prediction interval. The Pearson correlations coefficients (r) and significance level are presented on each graph.

FIG. 4 shows ROC plots for predicting GA>70 days using serum PAPP-A (dimeric PAPP-A+PAPP-A/proMBP) as measured using the 4-Ansh Labs assay, proMBP, and PAPP-A2 concentrations.

FIG. 5A shows a scatterplot of serum PAPP-A concentration in logarithmic format measured using 3-Creative Diagnosis and 4-Ansh Labs assays and converted to mIU/mL using each respective assay's recommended formula. The interrupted perpendicular grey lines mark the optimal cut-off points of the two assays, which differ as noted on the graph. The Pearson correlations coefficients (r) and significance level are also included. FIG. 5B shows passing-Bablok regression depicting the deviation of agreement between the two immunoassays even after the conversion formula is used. The interrupted grey line marks the line of perfect agreement. Also shown is the Passing Bablok regression line with the slope and intercept noted in the left corner of the graph (slope: 0.311, intercept: −78.074).

FIG. 6 shows the distribution of ultrasound-determined GA across an intended-use population of patients (n=264) seeking a medical abortion (MA).

FIGS. 7A-7B show ROC plots for predicting gestational age (GA)>70 days in the intended-use population using serum PAPP-A concentration measured with the 4-Ansh Labs (FIG. 7A) and the 1-R&D Systems immunoassay (FIG. 7B).

FIGS. 8A-8B show serum PAPP-A concentration (y-axes) measured in the intended-use population with the 4-Ansh Labs (FIG. 8A) and 1-R&D Systems immunoassays (FIG. 8B). Serum PAPP-A concentrations are plotted against GA (x-axes), with both variables expressed in base 10 logarithmic format. On each graph, the horizontal interrupted grey lines mark the respective assay's optimal cut-off point, which is also noted to the right of the line. The vertical interrupted grey line marks the GA cut-off of 70 days (10 weeks). Bracket 1 marks the respective assay's standard curve range using dilution protocols recommended by the manufacturer. Bracket 2 marks additional range achieved using modified dilution protocols.

FIGS. 9A-9B show serum PAPP-A concentration (y-axes) measured in the intended-use population with the 4-Ansh Labs (FIG. 9A) and 1-R&D Systems immunoassays (FIG. 9B). Serum PAPP-A concentrations are plotted against gestational age (GA) (x-axes), with both variables expressed in base 10 logarithmic format. The fitted equations are also displayed. On each graph, the solid black line represents the regression line, the solid grey lines represent the 95% confidence interval, and the grey dotted lines represent the 95% prediction interval.

FIGS. 10A-10B show agreement plots between GA as determined by ultrasound and by serum PAPP-A concentration levels measured with the 4-Ansh Labs (FIG. 10A) and 1-R&D Systems immunoassays (FIG. 10B) in the intended-use population. Lin's concordance coefficients and associate 95% confidence intervals are also shown.

### DETAILED DESCRIPTION

Provided herein in some embodiments are methods, devices, kits, and articles of manufacture for detecting pregnancy-associated plasma protein-A (PAPP-A) in a biological sample obtained from a pregnant subject. In some embodiments, the biological sample is a whole blood, serum, or plasma sample. In some embodiments, the provided methods include preparing a test sample from a pregnant female subject. In some embodiments, the test sample is prepared by obtaining a biological sample from the pregnant female subject and diluting the obtained biological sample in a sample diluent. In some embodiments, the provided methods include detecting in the test sample one or more PAPP-A proteoforms using an immunoassay, for instance a sandwich ELISA or sandwich lateral flow assay. In some embodiments, the immunoassay includes contacting the test sample with an antibody capable of specifically binding to (1) homodimeric PAPP-A and (2) the heterotetrameric PAPP-A/proMBP complex. In some embodiments, the provided methods also include measuring the concentration of PAPP-A in the obtained biological sample.

Also provided herein in some embodiments are methods, devices, kits, and articles of manufacture for classifying or determining the gestational age (GA) of a pregnancy by detecting PAPP-A in a biological sample obtained from a pregnant subject. In some embodiments, the provided methods involve detecting PAPP-A using an immunoassay, for instance a sandwich ELISA or sandwich lateral flow assay. In some embodiments, the immunoassay includes contacting the test sample with a capture antibody and a detection antibody each capable of specifically binding to (1) homodimeric PAPP-A and (2) the heterotetrameric PAPP-A/proMBP complex. In some embodiments, the immunoassay includes assessing in a test sample derived from the biological sample the degree of a detectable signal indicative of PAPP-A in the test sample. In some embodiments, the detectable signal is produced by a detectable marker conjugated to the detection antibody. In some embodiments, the assessing is performed when the detection antibody is bound to the one or more PAPP-A proteoforms or a complex containing same.

In some embodiments, the degree of the detectable signal is compared to the degree of a detectable signal assessed using the immunoassay in a reference PAPP-A sample. In some embodiments, the reference PAPP-A sample comprises or consists essentially of the heterotetrameric PAPP-A/proMBP complex. In some embodiments, the provided methods and devices, kits, and articles of manufacture for performing same are used to classify the GA of a pregnancy as above or below a predetermined GA cutpoint based on comparing the degree of the detectable signal assessed from the test sample to the degree of the detectable signal from the reference PAPP-A sample. In some embodiments, the GA of the pregnancy is classified as less than the predetermined GA cutpoint if the degree of the detectable signal assessed from the test sample is lower than the degree of the detectable signal assessed from the reference PAPP-A sample. In some embodiments, the GA of the pregnancy is classified as greater than or equal to the predetermined GA cutpoint if the degree of the detectable signal assessed from the test sample is higher than the degree of the detectable signal assessed from the reference PAPP-A sample. Also provided herein in some embodiments are methods of selecting a pregnant subject as eligible for a prenatal care or prenatal clinical treatment based on the GA of the pregnancy as classified using any of the provided methods, as well as methods of performing the prenatal care or prenatal clinical treatment on the pregnant subject selected as eligible. In some embodiments, the pregnant subject is eligible for a prenatal care or prenatal clinical treatment if the GA of the pregnancy is classified as less than the predetermined GA cutpoint. In some embodiments, the pregnant subject is ineligible for a prenatal care or prenatal clinical treatment if the GA of the pregnancy is classified as less than the predetermined GA cutpoint.

In some embodiments, the GA of the pregnancy is determined by providing the degree of the detectable signal assessed from the test sample as input to a process that predicts GA using the provided degree. Thus, in some embodiments, the GA of a pregnancy is determined without comparison to a reference PAPP-A sample. In some embodiments, the process predicts GAs over a particular range, for instance between or between about 5 weeks and 40 weeks. Also provided herein in some embodiments are methods of selecting and/or performing a prenatal care or prenatal clinical treatment on a pregnant subject based on the GA of the pregnancy as determined using any of the provided methods.

Accurate pregnancy dating is critical for maternal and child health and for counseling on needed prenatal care or treatment, including for safe and effective abortion methods. Early obstetrical ultrasound (up to 13^{6/7} weeks of gestation) is currently considered the gold standard, and guidelines recommend using ultrasound along with the last menstrual period (LMP) in order to revise or determine the estimated due date. The main barrier to this approach is that LMP depends on recall accuracy and may not yield an accurate GA estimate in women who have irregular periods, use long term hormonal contraception, or are lactating. Further, ultrasound is a costly method, requiring sophisticated instrumentation and skilled health care personnel, and is not always available in low and middle income countries.

In view of these considerations, the provided embodiments offer means of reliably assessing, without ultrasound, whether pregnancy has (or has not) progressed beyond a particular GA of clinical interest. The provided methods are easy to perform, simple to interpret, inexpensive, and do not require specialized medical facilities or laboratories. In some embodiments, the provided methods provide a test that produces a binary result to distinguish GAs above and below a predetermined GA cutpoint. In some embodiments, the provided methods provide an estimate of GA. Thus, the provided methods obviate the need for ultrasound for determining GA, including in connection with making prenatal care or prenatal clinical treatment decisions, for instance before an early aspiration or outpatient medical abortion procedure.

The provided methods are based on observations that the amount or concentration of PAPP-A in a biological sample, e.g., a whole blood or serum sample, from a pregnant subject is indicative of GA as determined by ultrasound. PAPP-A is a metalloprotease that is synthesized by the human placenta and decidua. PAPP-A is active in homodimeric form, cleaving insulin-like growth factor binding proteins (IGFBPs) and thereby increasing insulin growth factor (IGF) bioavailability. PAPP-A is also found covalently bound to pro-major eosinophilic protein (proMBP) in a heterotetrameric 2:2 complex, in which form its enzymatic activity is inhibited.

The inventors of the present application found that GA was most highly correlated with serum concentrations of PAPP-A as measured by detecting both proteoforms of PAPP-A (i.e., detecting both homodimeric PAPP-A and the heterotetrameric PAPP-A/proMBP complex). As shown herein using an exemplary assay, high correlation was seen across samples from a wide range of GAs, including for GAs below 15 weeks and even below 40 days. These results provide compelling evidence supporting the use of an antibody that detects both PAPP-A proteoforms for purposes of classifying or determining GA using PAPP-A, including during the first trimester. Thus, the provided methods can be used to inform using estimated GA a variety of decisions regarding prenatal care or prenatal clinical treatments.

Based on these findings, the inventors also found that concentrations of serum PAPP-A as measured by detecting both PAPP-A proteoforms could be used to classify GAs above and below a clinically relevant GA cutpoint of 70 days (10 weeks) with greater than 90% specificity and 90% sensitivity. This GA cutpoint is at the end of embryogenesis and is relevant for a variety of clinical or prenatal care decisions. For instance, 10-week GA is the currently accepted eligibility limit for treatment with mifepristone and misoprostol. Thus, the provided methods can be used to identify subjects that are eligible for a medical abortion, such as eligible for treatment with mifepristone and misoprostol. The provided methods can also be used to inform decisions about medical abortion regimens based on GA, such as inform choices regarding the dosing of mifepristone and misoprostol. Furthermore, since embryotoxicity is of primary concern within the first 8-10 weeks of gestation, the provided methods can be used to identify pregnancies that have progressed beyond that timepoint in order to assess risk from potentially dangerous exposures. The provided methods can also be used to identify if a patient is eligible for certain prenatal screening tests conducted at 10 weeks or later, such as screening tests for trisomy 21 (Down syndrome).

Moreover, the inventors demonstrated the above results using less than 10 μL of serum sample per pregnant subject, a surprising achievement given the finding of low PAPP-A concentration during early GAs. While certain commercially available assays may require low volumes of sample, they also might simultaneously require high dilution of the samples, for instance as much as a 150-fold dilution. Such requirements would suggest that these assays expect high PAPP-A concentration in the original samples and that these assays did not contemplate measuring PAPP-A during early pregnancy. In contrast, the inventors of the present application demonstrated that PAPP-A during early GAs can be detected using low-volume samples that are diluted less, for instance only 5- or 50-fold diluted. Thus, the methods provided herein allow for reliable, inexpensive tests that accurately estimate GA during early pregnancy without requiring high amounts of sample or specialized facilities.

As also shown herein, the findings described above were further validated in a study examining serum PAPP-A concentration levels in an intended-use population of pregnant subjects seeking a medical abortion, many of them having a GA less than 10 weeks (73%, median GA of 63 days). These findings further indicate the promise of PAPP-A immunoassays for predicting GA. While the presence in serum of PAPP-A in a heterotetrameric complex with proMBP has been reported in pregnant subjects, the presence of homodimeric PAPP-A at higher concentrations during early gestation (e.g., between 7-13 weeks GA) may be possible. Thus, the detection of both PAPP-A proteoforms may be particularly important in an intended-use population that has lower GA, for instance a population seeking medical abortion. These needs are met by the methods provided herein, which by detecting both PAPP-A proteoforms, allows for the accurate determination of total PAPP-A concentration in pregnant subjects with lower suspected GA and therefore for the accurate determination, without the need for ultrasound, of GA based on said total PAPP-A concentrations. As shown herein, serum PAPP-A concentration levels as measured using an immunoassay detecting both homodimeric PAPP-A and the heterotetrameric PAPP-A/proMBP complex predicted GA with high specificity and sensitivity in the intended-use population. Moreover, the reduced dilution levels described above allowed for the expansion of the range of the immunoassay such that PAPP-A concentration levels in more subjects with low suspected GA could be determined. In contrast, an alternative assay used for comparison in the Examples herein could not be used to assess serum PAPP-A concentrations in more than half of the samples from the intended-use population, and GA based on PAPP-A concentrations measured by this alternative assay could not be accurately determined below a GA of 74 days. These findings further indicate inventors' development and optimization of methods for predicting GA in an intended-use population having an average GA less than 10 weeks, said optimization including but not limited to the detection of both PAPP-A proteoforms and a modified dilution protocol for processing additional samples (e.g., those with lower PAPP-A concentrations). In contrast, other studies investigating PAPP-A immunoassays in pregnant subjects often do not include or examine pregnant subjects having ultrasound-determined GA that is less than 10 weeks (see, e.g., Lambert-Messerlian et al., Journal of Medical Screening 2010, 17(3):109-113, which included only pregnant subjects having GA of 10 weeks or higher).

The inventors also found that other demographic and clinical variables were associated with GA. Specifically, the inventors found that higher GA was associated with increased likelihood of current tobacco use and higher body mass index (BMI) in pregnant subjects seeking a medical abortion. As shown herein, the inventors found that these variables could be applied with use of PAPP-A concentration in methods for predicting GA.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, gestational age (GA) refers to weeks or days of human development timed from fertilization to the current date, plus 14 days. A normal pregnancy can range from 38 to 42 weeks.

As used herein, and unless otherwise specified, the terms “isolated” and “purified” describe a composition of matter that has been removed from its native environment (e.g., the natural environment if it is naturally occurring), and thus is altered by the hand of man from its natural state so as to possess markedly different characteristics with regard to at least one of structure, function, and properties. An isolated protein or nucleic acid is distinct from the way it exists in nature and includes synthetic peptides and proteins.

As used herein, the term “proteoform” is used to refer to any molecular form in which the protein product of a single gene can be found. This definition encompasses the various different protein products that arise from the same gene as a result of genetic variation, alternative splicing, and posttranslational modifications. This definition also encompasses the various different molecular complexes, for instance protein complexes, in which protein products of a single gene can be found.

Reference herein to “PAPP-A” refers to all PAPP-A proteoforms, including homodimeric PAPP-A and the heterotetrameric PAPP-A/proMBP complex.

Reference herein to the amount of PAPP-A in a sample (e.g., the amount of PAPP-A in a biological sample from a pregnant subject or the amount of PAPP-A in a reference PAPP-A sample) refers to the combined amount of homodimeric PAPP-A and the heterotetrameric PAPP-A/proMBP complex present in the sample. Reference herein to the concentration of PAPP-A in a sample (e.g., the concentration of PAPP-A in a biological sample from a pregnant subject or the concentration of PAPP-A in a reference PAPP-A sample) refers to a concentration that is based on the combined amount of homodimeric PAPP-A and the heterotetrameric PAPP-A/proMBP complex present in the sample.

As used herein, “antibody” refers to immunoglobulins and immunoglobulin fragments, whether natural or partially or wholly synthetically, such as recombinantly, produced, including any fragment thereof containing at least a portion of the variable heavy chain and/or light chain region of the immunoglobulin molecule that is sufficient to form an antigen binding site and, when assembled, to specifically bind antigen. Hence, an antibody includes any protein having a binding domain that is homologous or substantially homologous to an immunoglobulin antigen-binding domain (antibody combining site). Typically, antibodies minimally include all or at least a portion of the variable heavy (V H) chain and/or the variable light (V L) chain. In general, the pairing of a V H and V L together form the antigen-binding site, although, in some cases, a single V H or V L domain is sufficient for antigen-binding. The antibody also can include all or a portion of the constant region. Hence, it is understood that reference to an antibody herein includes full-length antibody and antigen-binding fragments, including those that specifically bind to a respective placental protein as described. Antibodies include polyclonal antibodies or monoclonal antibodies. Antibody also includes synthetic antibodies or recombinantly produced antibodies. The term “immunoglobulin” (Ig) is used interchangeably with “antibody” herein.

Thus, the term antibody includes full-length antibodies and portions thereof including antibody fragments, wherein such contain a heavy chain or portion thereof and/or a light chain or portion thereof. An antibody can contain two heavy chains (which can be denoted H and H′) and two light chains (which can be denoted L and L′). Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (CH1, CH2 and CH3) for each of the α and γ chains and four C_{H} domains (CH1, CH2, CH3 and CH4) for μ and ε isotypes. Each L chain has at the N-terminus, a variable domain (V_{L}) followed by a constant domain (C_{L}) at its other end. In some cases, the heavy chain can be a full-length immunoglobulin heavy chain or a portion thereof sufficient to form an antigen binding site (e.g., heavy chains include, but are not limited to, VH chains, VH-CH1 chains and VH-CH1-CH2-CH3 chains), and/or each light chain can be a full-length light chain or a portion thereof sufficient to form an antigen binding site (e.g., light chains include, but are not limited to, VL chains and VL-CL chains). For the structure and properties of the different classes of antibodies, see e.g., Basic and Clinical Immunology, 8th Edition, Daniel P. Sties, Abba I. Ten and Tristram G. Parsolw (eds), Appleton & Lange, Norwalk, C T, 1994, page 71 and Chapter 6. each heavy chain (H and H′) pairs with one light chain (L and L′, respectively).

The terms “full-length antibody,” “intact antibody” or “whole antibody” are used interchangeably to refer to an antibody in its substantially intact form, as opposed to an antibody fragment. A full-length antibody is an antibody typically having two full-length heavy chains (e.g., VH-CH1-CH2-CH3 or VH-CH1-CH2-CH3-CH4) and two full-length light chains (VL-CL) and hinge regions, such as antibodies produced from mammalian species (e.g. human, mouse, rat, rabbit, non-human primate, etc.) by antibody secreting B cells and antibodies with the same domains that are produced synthetically. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, the antigen binding and/or the variable region of the intact antibody. Antibody fragments, include, but are not limited to, Fab fragments, Fab′ fragments, F(ab′)₂ fragments, F_{V} fragments, disulfide-linked F_{V}s (dsF_{V}), Fd fragments, Fd′ fragments; diabodies; linear antibodies (see U.S. Pat. No. 5,641,870, Example 2; Zapata et al., Protein Eng. 8(10): 1057-1062 [1995]); single-chain antibody molecules, including single-chain F_{V}s (scF_{V}) or single-chain Fabs (scFab); antigen-binding fragments of any of the above and multispecific antibodies from antibody fragments.

A “monoclonal antibody” (mAb) refers to a homogeneous antibody population wherein the monoclonal antibody is comprised of amino acids (naturally occurring and non-naturally occurring) that are involved in the selective binding of an epitope. In particular, the CDRs of the monoclonal antibody are identical in all the molecules of the population. MAbs contain an antigen binding site capable of immunoreacting with a particular epitope of the antigen characterized by a unique binding affinity for it. The term “monoclonal antibody” encompasses not only intact monoclonal antibodies and full-length monoclonal antibodies, but also fragments thereof as described above.

As used herein, the terms “specific binding,” or “specifically binds” is the ability of a binding molecule, such as an antibody or an antigen-binding fragment, to preferentially bind an antigen in a complex mixture of proteins and/or macromolecules. A binding molecule is said to exhibit “specific binding” or “preferential binding” if it reacts or associates more frequently, more rapidly, with greater duration and/or with greater affinity with a particular cell or target antigen than it does with alternative cells or target antigens. A binding molecule specifically binds or preferentially binds to a target if it binds with greater affinity, avidity, more readily, and/or with greater duration than it binds to other substances. In some aspects, specific binding can refer to the non-covalent interactions of the type that occur between an immunoglobulin molecule and an antigen for which the immunoglobulin is specific. It is understood that specific binding or preferential binding does not necessarily require (although it can include) exclusive binding. Various known methods can be used to quantify or assess binding. The strength, or affinity of binding interactions can be expressed in terms of the dissociation constant (K_{d}) of the interaction, wherein a smaller K_{d} represents a greater affinity. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity and is equal to the dissociation constant K_{d}. (See, generally, Davies et al. (1990) Annual Rev Biochem 59:439-473). A binding molecule, such as an antibody or antigen binding fragment, is said to specifically bind, when the binding constant (K_{d}) is ≤1 μM, for example, in some embodiments ≤100 nM, in some embodiments ≤10 nM, and in some embodiments ≤100 pM to about 1 pM, as measured by assays such as radioligand binding assays or similar assays known to those skilled in the art.

As used herein, detection includes methods that permit visualization (by eye or equipment) of a protein. A protein can be visualized using an antibody specific to the protein. Detection of a protein can also be facilitated by fusion of a protein with a tag including an epitope tag or label.

The term “label” means any moiety which can be attached or linked, directly or indirectly, to an antibody and that functions to: (i) provide a detectable signal; (ii) interact with a second label to modify the detectable signal provided by the first or second label, e.g. FRET (fluorescence resonance energy transfer); (iii) stabilize interactions or increase affinity of binding, with antigen or ligand; (iv) affect mobility, e.g. electrophoretic mobility, or cell-permeability, by charge, hydrophobicity, shape, or other physical parameters, or (v) provide a capture moiety, to modulate ligand affinity, antibody/antigen binding, or ionic complexation.

As used herein, a “solid phase binding assay” refers to an in vitro assay in which an antigen is contacted with a ligand, where one of the antigen or ligand are bound to a solid support. Upon antigen-ligand interaction, the unwanted or non-specific components can be removed (e.g. by washing) and the antigen-ligand complex detected.

By “solid support” is meant a non-aqueous matrix to which an antibody according to the provided disclosure can adhere or attach. For example, solid supports include, but are not limited to, a microtiter plate, a membrane (e.g., nitrocellulose), a bead, a dipstick, a thin-layer chromatographic plate, or other solid medium.

The term “composition” refers to any mixture of two or more products, substances, or compounds, including antibodies. It may be a solution, a suspension, liquid, powder, a paste, aqueous, non-aqueous or any combination thereof. The preparation is generally in such form as to permit the biological activity of the active ingredient (e.g. antibody) to be effective.

As used herein, combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a kit is a packaged combination that optionally includes other elements, such as additional reagents and instructions for use of the combination or elements thereof, for a purpose including, but not limited to, detection, diagnosis, and/or assessment of a biological activity or property.

As used herein, an “article of manufacture” is a product that is made and sold. As used throughout this application, the term is intended to encompass any of the antibodies provided herein, including compositions and combinations thereof, contained in articles of packaging.

### II. Methods of Detecting or Measuring the Concentration of PAPP-A

In some embodiments, the provided methods involve detecting PAPP-A (i.e., detecting both homodimeric PAPP-A and the heterotetrameric PAPP-A/proMBP complex) in a biological sample obtained from a pregnant female subject. In some embodiments, the provided methods further involve measuring the amount or concentration of PAPP-A in the biological sample obtained from the pregnant female subject (i.e., measuring the combined amount of homodimeric PAPP-A and the heterotetrameric PAPP-A/proMBP complex present in the biological sample, or the concentration based on same). In some embodiments, the provided methods involving detecting PAPP-A in a test sample derived from the biological sample. In some embodiments, the provided methods involve preparing the test sample, for instance by diluting the biological sample in the sample diluent. In some embodiments, the provided methods are performed using an immunoassay, for instance a sandwich ELISA or a sandwich lateral flow assay. In some embodiments, the immunoassay involves contacting the test sample with one or more antibodies capable of specifically binding to homodimeric PAPP-A and the heterotetrameric PAPP-A/proMBP complex.

A. Biological Samples

In some embodiments, the methods are performed in vitro. In some embodiments, the biological sample is an isolated sample. In some embodiments, the biological sample is obtained or isolated from the pregnant subject. In some embodiments, the provided methods involve obtaining or isolating the biological sample from the pregnant subject. In some embodiments, the pregnant subject is human.

In some embodiments, the GA of the pregnant female subject is suspected to be within a certain GA range when the biological sample is obtained. In some embodiments, the GA of the pregnant female subject is suspected to be between or between about 5 weeks and 40 weeks, inclusive, when the biological sample is obtained. In some embodiments, the GA of the pregnant female subject is suspected to be between or between about 5 weeks and 35 weeks, inclusive, when the biological sample is obtained. In some embodiments, the GA of the pregnant female subject is suspected to be between or between about 5 weeks and 30 weeks, inclusive, when the biological sample is obtained. In some embodiments, the GA of the pregnant female subject is suspected to be between or between about 5 weeks and 25 weeks, inclusive, when the biological sample is obtained. In some embodiments, the GA of the pregnant female subject is suspected to be between or between about 5 weeks and 20 weeks, inclusive, when the biological sample is obtained. In some embodiments, the GA of the pregnant female subject is suspected to be between or between about 5 weeks and 15 weeks, inclusive, when the biological sample is obtained. In some embodiments, the GA of the pregnant female subject is suspected to be between or between about 5 weeks and 14 weeks, inclusive, when the biological sample is obtained. In some embodiments, the GA of the pregnant female subject is suspected to be between or between about 5 weeks and 13 weeks, inclusive, when the biological sample is obtained. In some embodiments, the GA of the pregnant female subject is suspected to be between or between about 5 weeks and 12 weeks, inclusive, when the biological sample is obtained. In some embodiments, the GA of the pregnant female subject is suspected to be between or between about 5 weeks and 11 weeks, inclusive, when the biological sample is obtained. In some embodiments, the GA of the pregnant female subject is suspected to be between or between about 5 weeks and 10 weeks, inclusive, when the biological sample is obtained. In some embodiments, the GA of the pregnant female subject is suspected to be between or between about 5 weeks and 9 weeks, inclusive, when the biological sample is obtained.

In some embodiments, when the biological sample is obtained, the GA of the pregnant female subject is suspected to be between or between about 35 days and 70 days, inclusive. In some embodiments, when the biological sample is obtained, the GA of the pregnant female subject is suspected to be between or between about 35 days and 69 days, inclusive. In some embodiments, when the biological sample is obtained, the GA of the pregnant female subject is suspected to be between or between about 35 days and 68 days, inclusive. In some embodiments, when the biological sample is obtained, the GA of the pregnant female subject is suspected to be between or between about 35 days and 67 days, inclusive. In some embodiments, when the biological sample is obtained, the GA of the pregnant female subject is suspected to be between or between about 35 days and 66 days, inclusive. In some embodiments, when the biological sample is obtained, the GA of the pregnant female subject is suspected to be between or between about 35 days and 65 days, inclusive. In some embodiments, when the biological sample is obtained, the GA of the pregnant female subject is suspected to be between or between about 35 days and 64 days, inclusive. In some embodiments, when the biological sample is obtained, the GA of the pregnant female subject is suspected to be between or between about 35 days and 63 days, inclusive.

In some embodiments, the obtained biological sample is a bodily fluid sample. In some embodiments, the obtained biological sample is a blood sample. In some embodiments, the obtained biological sample is a whole blood sample. In some embodiments, the obtained biological sample is a serum sample. In some embodiments, the obtained biological sample is a plasma sample.

In some embodiments, the obtained biological sample is of a certain volume. In some embodiments, the obtained biological sample is a blood sample, for instance a whole blood sample, and the obtained biological sample has a volume that can be collected by a finger stick test. In some embodiments, the obtained biological sample has a volume less than or equal to or less than or equal to about 10 In some embodiments, the obtained biological sample has a volume between or between about 0.5 and 10 inclusive. In some embodiments, the obtained biological sample has a volume between or between about 0.5 and 9 μL, 0.5 and 8 μL, 0.5 and 7 μL, 0.5 and 6 μL, 0.5 and 5 μL, 0.5 and 4 μL, 0.5 and 3 μL, 0.5 and 2 μL, 0.5 and 1.5 μL, 0.5 and 1 μL, 1 and 10 μL, 1 and 9 μL, 1 and 8 μL, 1 and 7 μL, 1 and 6 μL, 1 and 5 μL, 1 and 4 μL, 1 and 3 μL, 1 and 2 μL, 1 and 1.5 μL, 1.5 and 10 μL, 1.5 and 9 μL, 1.5 and 8 μL, 1.5 and 7 μL, 1.5 and 6 μL, 1.5 and 5 μL, 1.5 and 4 μL, 1.5 and 3 μL, 1.5 and 2 μL, 2 and 10 μL, 2 and 9 μL, 2 and 8 μL, 2 and 7 μL, 2 and 6 μL, 2 and 5 μL, 2 and 4 μL, 2 and 3 μL, 3 and 10 μL, 3 and 9 μL, 3 and 8 μL, 3 and 7 μL, 3 and 6 μL, 3 and 5 μL, 3 and 4 μL, 4 and 10 μL, 4 and 9 μL, 4 and 8 μL, 4 and 7 μL, 4 and 6 μL, 4 and 5 μL, 5 and 10 μL, 5 and 9 μL, 5 and 8 μL, 5 and 7 μL, 5 and 6 μL, 6 and 10 μL, 6 and 9 μL, 6 and 8 μL, 6 and 7 μL, 7 and 10 μL, 7 and 9 μL, 7 and 8 μL, 8 and 10 μL, 8 and 9 μL, or 9 and 10 μL, each inclusive. In some embodiments, the obtained biological sample has a volume or of about 1 μL, 2 μL, 3 μL, 4 μL, 5 μL, 6 μL, 7 μL, 8 μL, 9 μL, or 10 μL. In some embodiments, the obtained biological sample has a volume or of about 1 In some embodiments, the obtained biological sample has a volume or of about 2 μL. In some embodiments, the obtained biological sample has a volume or of about 5 μL. In some embodiments, the obtained biological sample has a volume or of about 10 μL.

B. Test Sample Preparation

In some embodiments, the obtained biological sample is used directly in the methods provided herein after obtaining it from the pregnant subject. In other embodiments, the provided methods involve preparing a test sample using the obtained biological sample before any steps of detecting or measuring the amount or concentration of PAPP-A. The obtained biological sample can be any as described in Section II-A.

In some embodiments, the test sample is prepared by diluting the obtained biological sample, for instance by diluting the obtained biological sample in a sample diluent. In some embodiments, the provided methods include preparing a test sample by diluting the obtained biological sample. In some embodiments, the volume of the biological sample is diluted between or between about 2-fold and 1500-fold, such as between or between about 2-fold and 1400-fold, 2-fold and 1300-fold, 2-fold and 1200-fold, 2-fold and 1100-fold, 2-fold and 1000-fold, 2-fold and 900-fold, 2-fold and 800-fold, 2-fold and 700-fold, 2-fold and 600-fold, 2-fold and 500-fold, 2-fold and 400-fold, 2-fold and 300-fold, 2-fold and 200-fold, 2-fold and 150-fold, 2-fold and 100-fold, 2-fold and 50-fold, 5-fold and 1500-fold, 5-fold and 1400-fold, 5-fold and 1300-fold, 5-fold and 1200-fold, 5-fold and 1100-fold, 5-fold and 1000-fold, 5-fold and 900-fold, 5-fold and 800-fold, 5-fold and 700-fold, 5-fold and 600-fold, 5-fold and 500-fold, 5-fold and 400-fold, 5-fold and 300-fold, 5-fold and 200-fold, 5-fold and 150-fold, 5-fold and 100-fold, or 5-fold and 50-fold, each inclusive. In some embodiments, the volume of the biological sample is diluted between or between about 2-fold and 100-fold, inclusive. In some embodiments, the volume of the biological sample is diluted between or between about 2-fold and 50-fold, inclusive. In some embodiments, the volume of the biological sample is diluted between or between about 5-fold and 100-fold, inclusive. In some embodiments, the volume of the biological sample is diluted between or between about 5-fold and 50-fold, inclusive. In some embodiments, the volume of the biological sample is diluted or diluted about 2-fold. In some embodiments, the volume of the biological sample is diluted or diluted about 5-fold. In some embodiments, the volume of the biological sample is diluted or diluted about 10-fold. In some embodiments, the volume of the biological sample is diluted or diluted about 25-fold. In some embodiments, the volume of the biological sample is diluted or diluted about 50-fold.

In some embodiments, the biological sample is diluted using a sample diluent. Any sample diluent suitable for use in an immunoassay can be used. In some embodiments, the sample diluent includes deionized water and buffered saline, such as Phosphate Buffered Saline or Tris Buffered Saline. In some embodiments, the sample diluent further includes a detergent. Any detergent suitable for use in an immunoassay can be used. In some embodiments, the detergent is a nonionic detergent, for instance Triton X100, NP40, or Tween 20. In some embodiments, the sample diluent does not include a metal chelator. In some embodiments, the sample diluent does not include EDTA.

In some embodiments, the test sample is prepared using further processing steps. These further processing steps can be before or after diluting the obtained biological sample. Further processing steps can include washing in a buffer, incubation, centrifugation, filtration, immunoprecipitation, adsorption, and/or addition of agents that remove contaminants that can interfere with detecting the binding between PAPP-A and an antibody capable of specifically binding to PAPP-A. In some embodiments, the obtained biological sample is a whole blood sample, and the test sample is prepared by also separating plasma from the serum.

C. Immunoassays

In some embodiments, the provided methods involve detecting or measuring the amount or concentration of PAPP-A in the obtained biological sample using an immunoassay. In some embodiments, the immunoassay is immunohistochemistry (IHC); flow cytometry; or Western blot. In some embodiments, the immunoassay is a colorimetric assay. In some embodiments, the immunoassay is a solid-phase immunoassay. In some embodiments, the immunoassay is an enzyme linked immunosorbent assay (ELISA), for instance a sandwich ELISA or competitive ELISA. In some embodiments, the immunoassay is a sandwich ELISA. In some embodiment, the immunoassay is a lateral flow assay. In some embodiment, the immunoassay is a sandwich lateral flow assay.

In some embodiments, the immunoassay is used to detect in a test sample one or more PAPP-A proteoforms. The test sample can be any as described in Section II-B. In some embodiments, the one or more PAPP-A proteoforms are one or more isolated PAPP-A proteoforms.

In some embodiments, the immunoassay includes the step of contacting a test sample from a pregnant subject with one or more antibodies that specifically bind to PAPP-A. In some embodiments, at least one of the one or more antibodies specifically binds to homodimeric PAPP-A. In some embodiments, at least one of the one or more antibodies specifically binds to the heterotetrameric PAPP-A/proMBP complex. In some embodiments, the one or more antibodies specifically bind to homodimeric PAPP-A and the heterotetrameric PAPP-A/proMBP complex. In some embodiments, the one or more antibodies bind to homodimeric PAPP-A and the heterotetrameric PAPP-A/proMBP complex equimolarly or about equimolarly.

In some embodiments, the one or more antibodies do not specifically bind to proMBP. In some embodiments, the one or more antibodies do not cross-react with proMBP. In some embodiments, the one or more antibodies do not cross-react with proMBP at up to twice the physiological concentrations of proMBP. In some embodiments, the one or more antibodies do not specifically bind to PAPP-A2. In some embodiments, the one or more antibodies do not cross-react with PAPP-A2. In some embodiments, the one or more antibodies do not cross-react with PAPP-A2 at up to twice the physiological concentrations of PAPP-A2. In some embodiments, the one or more antibodies do not specifically bind to MMP-9. In some embodiments, the one or more antibodies do not cross-react with MMP-9. In some embodiments, the one or more antibodies do not cross-react with MMP-9 at up to twice the physiological concentrations of MMP-9. In some embodiments, the physiological concentrations are with respect to serum concentration levels. In some embodiments, the physiological concentrations are with respect to serum concentration levels in pregnant subjects.

In some embodiments, the one or more antibodies specifically bind to a PAPP-A subunit of homodimeric PAPP-A. In some embodiments, the one or more antibodies specifically bind to a PAPP-A subunit of the heterotetrameric PAPP-A/proMBP complex. In some embodiments, the one or more antibodies specifically bind to a PAPP-A subunit of homodimeric PAPP-A and specifically binds to a PAPP-A subunit of the heterotetrameric PAPP-A/proMBP complex.

In some embodiments, the one or more antibodies do not bind the metal chelating region of PAPP-A.

In some embodiments, the immunoassay includes the step of contacting the test sample from the pregnant subject with an antibody capable of specifically binding to (1) homodimeric PAPP-A and (2) the heterotetrameric PAPP-A/proMBP complex. In some embodiments, the antibody is capable of binding to (1) homodimeric PAPP-A and (2) the heterotetrameric PAPP-A/proMBP complex equimolarly or about equimolarly. In some embodiments, the contacting is performed under conditions to form a complex comprising the antibody and the one or more PAPP-A proteoforms. In some embodiments, PAPP-A is detected by detecting the complex that includes the antibody and the one or more PAPP-A proteoforms.

In some embodiments, the antibody does not specifically bind to proMBP. In some embodiments, the antibody does not cross-react with proMBP. In some embodiments, the antibody does not cross-react with proMBP at up to twice the physiological concentrations of proMBP. In some embodiments, the antibody does not specifically bind to PAPP-A2. In some embodiments, the antibody does not cross-react with PAPP-A2. In some embodiments, the antibody does not cross-react with PAPP-A2 at up to twice the physiological concentrations of PAPP-A2. In some embodiments, the antibody does not specifically bind to MMP-9. In some embodiments, the antibody does not cross-react with MMP-9. In some embodiments, the antibody does not cross-react with MMP-9 at up to twice the physiological concentrations of MMP-9. In some embodiments, the physiological concentrations are with respect to serum concentration levels. In some embodiments, the physiological concentrations are with respect to serum concentration levels in pregnant subjects.

In some embodiments, the antibody specifically binds to a PAPP-A subunit of homodimeric PAPP-A. In some embodiments, the antibody specifically binds to a PAPP-A subunit of the heterotetrameric PAPP-A/proMBP complex. In some embodiments, antibody specifically binds to a PAPP-A subunit of homodimeric PAPP-A and specifically binds to a PAPP-A subunit of the heterotetrameric PAPP-A/proMBP complex.

In some embodiments, the antibody does not bind the metal chelating region of PAPP-A.

In some embodiments, the antibody is a capture antibody. In some embodiments, the antibody, e.g., the capture antibody, is unlabeled.

In some embodiments, the antibody, e.g., the capture antibody, is immobilized on a solid support. The antibody, e.g., the capture antibody, can be immobilized directly or indirectly on the solid support. In some embodiments, the solid support is a bead, column, array, assay plate, microwell, cartridge, stick, filter, or strip. In some embodiments, the solid support is formed of glass (e.g., controlled pore glass), polysaccharides (e.g., agarose), polyacrylamides, polystyrene, polyvinyl alcohol, nitrocellulose, cellulose, nylon, silicones, or other materials well known in the art to which antibodies or antibody fragments can be directly or indirectly attached or immobilized.

In some embodiments, prior to detecting the complex, the immunoassay further includes contacting the test sample with a detection antibody. In some embodiments, the contacting is performed under conditions to form a complex that includes the capture antibody, the one or more PAPP-A proteoforms, and the capture antibody.

In some embodiments, the capture antibody is unlabeled, and the detection antibody is directly or indirectly labeled for detection. In some examples, the second antibody is detected using a secondary reagent, such as by a secondary antibody reagent that binds to the second antibody and that is coupled to a detectable label capable of producing a detectable signal, such as a fluorescent probe or detectable enzyme, such as horseradish peroxidase. In some embodiments, the detection antibody is conjugated directly to a detectable label capable of producing a detectable signal. In some embodiments, the detectable label is a colored particle, for instance latex or gold. In some embodiments, the detectable label is fluorescent or magnetic. In some embodiments, the detectable label is a detectable enzyme. In some embodiments, the detectable label is or includes horseradish peroxidase. In some embodiments, the complex that includes the capture antibody, the one or more PAPP-A proteoforms, and the detection antibody is detected by assessing the degree of the detectable signal. Reference to the degree of the detectable signal with reference to a detectable signal produced by a detectable label can refer to the amount, level, magnitude, or extent of a signal, for instance the intensity or strength of the signal. This can include any degree as determined or measured based on visual inspection or using a system, machine or device that is able to measure or determine or assess a detectable signal.

In some embodiments, the complex that includes the capture antibody, the one or more PAPP-A proteoforms, and the detection antibody is detected by the addition of a detection substrate, such as ABTS (2,2′-Azinobis [3-ethylbenzothiazoline-6-sulfonic acid]-diammonium salt), OPD (o-phenylenediamine dihydrochloride), or TMB (3,3′,5,5′-tetramethylbenzidine) detection substrate and subsequently assessing the degree of the detectable signal. Other labels or detection reagents suitable for use with particular labels are well known to a skilled artisan and will vary based on the type of immunoassay employed, for instance if the immunoassay is a sandwich ELISA or a sandwich lateral flow assay. For instance, in some embodiments the immunoassay is a sandwich ELISA, and the detectable label is or includes horseradish peroxidase. In some embodiments, the immunoassay is a sandwich lateral flow assay, and the detectable label is a colored particle such as latex or gold.

In some embodiments, the detection antibody is capable of specifically binding to (1) homodimeric PAPP-A and (2) the heterotetrameric PAPP-A/proMBP complex. In some embodiments, the detection antibody is capable of binding to (1) homodimeric PAPP-A and (2) the heterotetrameric PAPP-A/proMBP complex equimolarly or about equimolarly.

In some embodiments, the detection antibody does not specifically bind to proMBP. In some embodiments, the detection antibody does not cross-react with proMBP. In some embodiments, the detection antibody does not cross-react with proMBP at up to twice the physiological concentrations of proMBP. In some embodiments, the detection antibody does not specifically bind to PAPP-A2. In some embodiments, the detection antibody does not cross-react with PAPP-A2. In some embodiments, the detection antibody does not cross-react with PAPP-A2 at up to twice the physiological concentrations of PAPP-A2. In some embodiments, the detection antibody does not specifically bind to MMP-9. In some embodiments, the detection antibody does not cross-react with MMP-9. In some embodiments, the detection antibody does not cross-react with MMP-9 at up to twice the physiological concentrations of MMP-9. In some embodiments, the physiological concentrations are with respect to serum concentration levels. In some embodiments, the physiological concentrations are with respect to serum concentration levels in pregnant subjects.

In some embodiments, the detection antibody specifically binds to a PAPP-A subunit of homodimeric PAPP-A. In some embodiments, the detection antibody specifically binds to a PAPP-A subunit of the heterotetrameric PAPP-A/proMBP complex. In some embodiments, the detection antibody specifically binds to a PAPP-A subunit of homodimeric PAPP-A and specifically binds to a PAPP-A subunit of the heterotetrameric PAPP-A/proMBP complex.

In some embodiments, the detection antibody does not bind the metal chelating region of PAPP-A.

In some embodiments, the capture antibody is a monoclonal antibody. In some embodiments, the detection antibody is a monoclonal antibody or a polyclonal antibody. In some embodiments, the capture antibody is different from the detection antibody, such as binds to a distinct or non-overlapping epitope compared to the detection antibody. In some embodiments, the capture antibody does not compete for binding to PAPP-A with the detection antibody. In some embodiments, the capture and detection antibodies can be part of an antibody pair for use in detection or measuring the amount or concentration of PAPP-A in the biological sample.

In some embodiments, the capture antibody and the detection antibody are capable of binding to homodimeric PAPP-A and the heterotetrameric PAPP-A/proMBP complex. In some embodiments, the capture antibody and the detection antibody are capable of binding to homodimeric PAPP-A and the heterotetrameric PAPP-A/proMBP complex equimolarly or about equimolarly.

In some embodiments, the capture antibody and the detection antibody do not specifically bind to proMBP. In some embodiments, the capture antibody and the detection antibody do not cross-react with proMBP. In some embodiments, the capture antibody and the detection antibody do not cross-react with proMBP at up to twice the physiological concentrations of proMBP. In some embodiments, the capture antibody and the detection antibody do not specifically bind to PAPP-A2. In some embodiments, the capture antibody and the detection antibody do not cross-react with PAPP-A2. In some embodiments, the capture antibody and the detection antibody do not cross-react with PAPP-A2 at up to twice the physiological concentrations of PAPP-A2. In some embodiments, the capture antibody and the detection antibody do not specifically bind to MMP-9. In some embodiments, the capture antibody and the detection antibody do not cross-react with MMP-9. In some embodiments, the capture antibody and the detection antibody do not cross-react with MMP-9 at up to twice the physiological concentrations of MMP-9. In some embodiments, the physiological concentrations are with respect to serum concentration levels. In some embodiments, the physiological concentrations are with respect to serum concentration levels in pregnant subjects.

In some embodiments, the capture antibody and the detection antibody specifically bind to a PAPP-A subunit of homodimeric PAPP-A. In some embodiments, the capture antibody and the detection antibody specifically bind to a PAPP-A subunit of the heterotetrameric PAPP-A/proMBP complex. In some embodiments, the capture antibody and the detection antibody specifically bind to a PAPP-A subunit of homodimeric PAPP-A and specifically binds to a PAPP-A subunit of the heterotetrameric PAPP-A/proMBP complex.

In some embodiments, the capture antibody and the detection antibody do not bind the metal chelating region of PAPP-A.

In some embodiments, the test sample is contacted with the capture antibody and the detection antibody simultaneously. In some embodiments, the test sample is contacted with the capture antibody and the detection antibody sequentially. In some embodiments, the test sample is contacted with the capture antibody prior to being contacted with the detection antibody, for instance in the case of a sandwich ELISA. In some embodiments, the test sample is contacted with the detection antibody prior to being contacted with the capture antibody, for instance in the case of a sandwich lateral flow assay.

In some embodiments, the immunoassay is a sandwich ELISA, and using the immunoassay to detect or measure the amount or concentration of PAPP-A (i.e., the amount or concentration of homodimeric PAPP-A and the heterotetrameric PAPP-A/proMBP complex) includes (a) contacting the test sample from the pregnant subject with the capture antibody to capture or bind the one or more PAPP-A proteoforms (e.g., homodimeric PAPP-A and/or the heterotetrameric PAPP-A/proMBP complex) in the sample (e.g., under conditions to form a first complex that includes the capture antibody and the one or more PAPP-A proteoforms); and (b) contacting the first complex with the detection antibody that binds to the first complex (e.g., under conditions to form a second complex that includes the capture antibody, the one or more PAPP-A proteoforms, and the detection antibody. In some embodiments, the second complex is detected by assessing the degree of the detectable signal produced by the detectable label conjugated to the detection antibody.

In some embodiments, the sample is mixed with the capture antibody to produce a mixture, and the mixture is subsequently applied to a solid support or a device comprising a solid support. In other embodiments, the test sample is mixed with the capture antibody in the presence of, on, or in a solid support or a device comprising the solid support. In some embodiments, the capture antibody is immobilized to the solid support (e.g., coated to wells of a microtiter plate). In some embodiments, the capture antibody is directly or indirectly attached to the solid support. In some embodiments, the capture antibody is conjugated to a reagent to facilitate its immobilization on the solid support. In some embodiments, the capture antibody is conjugated to biotin, and the solid support has immobilized thereon or is coated with a biotin-binding reagent, such as streptavidin.

In some embodiments, the test sample and the capture antibody are incubated under conditions to form a first complex containing the capture antibody and the one or more PAPP-A proteoforms. The incubation can be for a time that is suitable to allow the one or more PAPP-A proteoforms in the test sample to contact the capture antibody, such as for at least or at least about 30 seconds, 1 minute, 5 minutes, 10 minutes, 15 minutes, 20 minutes, 30 minutes, 45 minutes, 1 hour, 2 hours, 3 hours, 6 hours, or 12 hours or more but no more than about 24 hours after contacting the test sample with the capture antibody. In some embodiments, the contacting occurs at a temperature of from or from about 0° C. to about 50° C., such as typically 2° C. to 8° C. or 23° C. to 28° C. or 37° C. to 42° C. In some embodiments, after the contacting, the method includes one or more washing steps under conditions to retain PAPP-A proteoforms bound on the solid support and/or to separate or remove any unbound reagents or proteins of the sample not part of the first complex and/or that were not specifically bound by the capture antibody.

In some embodiments, the detection antibody is applied to or contacted with the first complex that includes the capture antibody and the one or more PAPP-A proteoforms and allowed to incubate under conditions to allow binding of the detection antibody to the one or more PAPP-A proteoforms in the first complex. In some embodiments, the incubations is for a time that is suitable to allow the detection antibody to contact the one or more PAPP-A proteoforms in the first complex with the capture antibody, such as for at least or at least about 30 seconds, 1 minute, 5 minutes, 10 minutes, 15 minutes, 20 minutes, 30 minutes, 45 minutes, 1 hour, 2 hours, 3 hours, 6 hours or 12 hours or more but no more than about 24 hours after contacting the first complex with the detection antibody. In some embodiments, the contacting occurs at a temperature of from or from about 0° C. to about 30° C., such as typically 2° C. to 8° C. or 23° C. to 28° C. In some embodiments, after the contacting with the detection antibody, the method furthers include one or more washing steps under conditions to retain binding of the detection antibody to the one or more PAPP-A proteoforms in the second complex and to remove any unbound second antibody.

In some embodiments, the immunoassay is a sandwich lateral flow assay, and using the immunoassay to detect or measure the amount or concentration of PAPP-A (i.e., the amount or concentration of homodimeric PAPP-A and the heterotetrameric PAPP-A/proMBP complex) includes (a) contacting the test sample from the pregnant subject with the detection antibody to capture or bind the one or more PAPP-A proteoforms (e.g., homodimeric PAPP-A and/or the heterotetrameric PAPP-A/proMBP complex) in the sample (e.g., under conditions to form a first complex that includes the detection antibody and the one or more PAPP-A proteoforms); and (b) contacting the first complex with the capture antibody that binds to the first complex (e.g., under conditions to form a second complex that includes the capture antibody, the one or more PAPP-A proteoforms, and the detection antibody. In some embodiments, the second complex is detected by assessing the degree of the detectable signal produced by the detectable label conjugated to the detection antibody.

In any of the provided embodiments, the detectable signal produced by the detection antibody or the detectable label conjugated to the detection antibody is then detected. In some embodiments, the detectable signal is detected visually. In some embodiments, the degree of the detectable signal is assessed visually. Detection methods can also include colorimetric, fluorescent, luminescent, or radioactive methods. In some embodiments, the detecting and assessing of the degree of the detectable signal is carried out using an instrument or system able to detect, measure, or quantify the degree of the detectable signal. In some embodiments, the instrument or system provides a readout of the degree of the detectable signal. The choice of detection method is dependent on the detectable label used. In some examples, a colorimetric reaction is used in which the detection antibody is conjugated to an enzyme, such as horseradish peroxidase (HRP), alkaline phosphatase, or other detectable enzyme.

In some embodiments, the amount or concentration of PAPP-A present in the biological sample is then determined using the degree of the detectable signal. In some such aspects, the amount or concentration of PAPP-A present in the biological sample is proportional to the degree of the detectable signal, e.g. color, produced in the test sample. In some embodiments, the provided methods involve quantifying the amount of signal produced. Methods for quantification of signals are well known in the art, such as through use of a luminometer, spectrophotometer, or a digital imaging instrument. In some embodiments, one or more standard samples are employed to quantitate or determine the amount or concentration of PAPP-A in the biological sample. In some embodiments, the standard samples include known concentrations (e.g., serial dilutions) of a recombinant or native form of PAPP-A. In some embodiments, the standard samples range in concentration between approximately 35 and 7500 ng/mL of PAPP-A. In some embodiments, the concentrations of the standard samples and the signals measured from the standard samples are used to calculate a standard curve. In some embodiments, the amount or concentration of PAPP-A in the biological sample is calculated as a relative amount by interpolating the signal measured from the test sample to the standard curve. In some embodiments, the fold dilution used to prepare the test sample is accounted for in determining the amount or concentration of PAPP-A in the biological sample. The amount of PAPP-A can be expressed as an absolute quantity, and the concentration of PAPP-A can be expressed in mass per unit volume, moles (e.g. nano moles) per unit volume, or as international units per unit volume. In some embodiments, the method employed is semi-quantitative.

In some embodiments, the provided methods include detecting homodimeric PAPP-A and the heterotetrameric PAPP-A/proMBP complex in a biological sample (e.g., whole blood sample) using a sandwich ELISA. In some embodiments, a test sample is prepared from the biological sample by diluting the biological sample in a sample diluent. In some aspects, the immunoassay includes (a) contacting the test sample from the pregnant subject with an immobilized capture antibody in order to detect one or more PAP-A proteoforms under conditions to form a first complex that includes the capture antibody and the one or more PAPP-A proteoforms, and then carrying out one or more optional wash steps; (b) contacting the first complex containing the bound PAPP-A proteoforms with a detection antibody conjugated to a detectable label capable of producing a detectable signal under conditions to form a second complex that includes the capture antibody, the one or more PAPP-A proteoforms, and the detection antibody, and then carrying out one or more optional wash steps; and (c) detecting PAPP-A bound by the detection antibody by detecting the detectable signal. In some embodiments, both the capture antibody and the detection antibody bind to homodimeric PAPP-A and the heterotetrameric PAPP-A/proMBP complex, for instance equimolarly or about equimolarly. In some embodiments, the amount or concentration of PAPP-A is determined by comparison of the degree of the detectable signal to a standard curve, such as a standard curve constructed using a series of known concentrations of a recombinant or native PAPP-A.

In some embodiments, the provided methods include detecting homodimeric PAPP-A and the heterotetrameric PAPP-A/proMBP complex in a biological sample (e.g., whole blood sample) using a sandwich lateral flow assay. In some embodiments, a test sample is prepared from the biological sample by diluting the biological sample in a sample diluent. In some aspects, the immunoassay includes (a) contacting the test sample from the pregnant subject with a detection antibody conjugated to a detectable label capable of producing a detectable signal in order to detect one or more PAPP-A proteoforms under conditions to form a first complex that includes the detection antibody and the one or more PAPP-A proteoforms; (b) contacting the first complex containing the bound PAPP-A proteoforms with an immobilized capture antibody under conditions to form a second complex that includes the capture antibody, the one or more PAPP-A proteoforms, and the detection antibody; and (c) detecting PAPP-A bound by the detection antibody by detecting the detectable signal. In some embodiments, both the capture antibody and the detection antibody bind to homodimeric PAPP-A and the heterotetrameric PAPP-A/proMBP complex, for instance equimolarly or about equimolarly. In some embodiments, the amount or concentration of PAPP-A is determined by comparison of the degree of the detectable signal to a standard curve, such as a standard curve constructed using a series of known concentrations of a recombinant or native PAPP-A.

Antibodies and immunoassays for PAPP-A are known and include commercial assays and kits for detecting PAPP-A. In particular, antibodies capable of specifically binding to both homodimeric PAPP-A and the heterotetrameric PAPP-A/proMBP complex are known to a skilled artisan.

Exemplary antibodies capable of specifically binding to both homodimeric PAPP-A and the heterotetrameric PAPP-A/proMBP complex and that can be used as the capture and detection antibody include antibodies available from Ansh Labs Catalog No. AB-301-AP015, Ansh Labs Catalog No. AB-301-AP016, Antibodies-Online Catalog No. ABIN349820, clone 5H9 (ThermoFisher Scientific Catalog No. MA1-46425), clone 10E1 (Abnova; Catalog No. MAB0570), clone A1 (ProSci Catalog No. 54-308), clone C1 (ProSci Catalog No. 54-309), clone 7A6 (MyBioSource Catalog No. MBS312644), clone 1.B.132 (US Biological Life Sciences Catalog No. P6000-82), and clone 9L574 (US Biological Life Sciences Catalog No. P6000-82). Exemplary first and second antibody pairs include clone 10E1 and clone 7A6; clone A1 and clone C1; and clone 1.B.132 and clone 9L574. In some embodiments, the first and second antibody pair is Ansh Catalog No. AB-301-AP015 and Ansh Catalog No. AB-301-AP016. Exemplary immunoassays include Ansh Labs Catalog No. AL-106.

In some embodiments, exemplary first and second antibody pairs that can be used as the capture and detection antibodies for detecting both homodimeric PAPP-A and the heterotetrameric PAPP-A/proMBP complex in methods herein include clone 10E2 (ThermoFisher Scientific Catalog No. MA1-22743) and clone 10E1; clone 4G11 (GeneTex Catalog No. GTX10192) and clone 3C8 (GeneTex Catalog No. GTX10194); and clone 4G11 and clone 10H9 (GeneTex Catalog No. GTX10195). Other exemplary antibodies for use in the present methods (e.g., that can be used as capture and detection antibodies) are also described in Qin et al., Clin. Chem. 2006, 52(3):398-404; Qin et al., Clin. Chem. 1997, 43(12):2323-2332; Gyrup et al., Clin. Chem. 2007, 53(5):947-954; Qin et al., Recent Advances in Prenatal Diagnosis for Aneuploidy, 1-3 May 1996, Amsterdam, p 13; and US 2007/0111254.

### III. Methods of Estimating Gestational Age Based on PAPP-A

In some embodiments, the provided methods involve estimating the gestational age (GA) of a pregnant subject by detecting PAPP-A in a biological sample obtained from the pregnant subject. In some embodiments, the biological sample is any as described in Section II-A.

In some embodiments, the provided methods include detecting in a test sample derived from the biological sample one or more PAPP-A proteoforms. In some embodiments, the methods involve preparing a test sample using the biological sample prior to the detecting. In some embodiments, the test sample is any as described in Section II-B, or is prepared using any of the methods described in Section II-B.

In some embodiments, the methods involve detecting PAPP-A in the biological sample using an immunoassay. In some embodiments, the detecting is performed using an immunoassay, for instance a sandwich ELISA or a sandwich lateral flow assay. Exemplary methods of detecting PAPP-A in the biological sample are described in Section II-C. In some embodiments, the immunoassay involves the use of one or more antibodies, for instance a capture antibody and a detection antibody, each capable of specifically binding to homodimeric PAPP-A and the heterotetrameric PAPP-A/proMBP complex.

In some embodiments, the immunoassay involves assessing the degree of a detectable signal indicative of the amount of PAPP-A in the test sample. In some embodiments, the detectable signal is produced by a detectable label conjugated to the detection antibody. In some embodiments, the detectable signal is assessed when the detection antibody is bound to the one or more PAPP-A proteoforms or is in a complex containing the one or more PAPP-A proteoforms. In some embodiments, the methods further involve measuring the amount or concentration of PAPP-A in the biological sample, for instance using the degree of the detectable signal.

In some embodiments, the methods further include the step of comparing the degree of the detectable signal from the test sample or the concentration of PAPP-A in the biological sample to that of a reference PAPP-A sample. In some embodiments, the methods further include the step of performing the immunoassay on the reference PAPP-A sample, for instance to detect or to measure the concentration of PAPP-A in the reference PAPP-A sample. In some embodiments, the methods further include the step of comparing the concentration of PAPP-A in the test sample to a predetermined concentration. In some embodiments, the concentration of PAPP-A in the reference PAPP-A sample or the predetermined concentration is associated with PAPP-A levels at a predetermined GA cutpoint. In some embodiments, the methods further include the step of classifying the GA of the pregnancy as less than or greater than or equal to the predetermined GA cutpoint based on comparison between the test sample and the reference PAPP-A sample or between the test sample and the predetermined concentration.

In other embodiments, the methods include the step of determining the GA of the pregnancy based on the degree of the detectable signal from the test sample or based on the amount or concentration of PAPP-A in the biological sample, wherein the degree of the detectable signal or the PAPP-A amount or concentration is used as a predictor of GA. In some embodiments, the degree of the detectable signal in the test sample or the amount or concentration of PAPP-A in the biological sample is provided as input to a process that uses the provided degree or PAPP-A amount or concentration as a predictor, e.g., a continuous predictor, of GA.

In some embodiments, the methods further include selecting or screening a subject for a certain prenatal care, prenatal clinical treatment, or other decision or action based on results of the GA estimation. In some embodiments, the methods further include carrying out the prenatal care, prenatal clinical treatment, or other decision or action on the pregnant subject based on the selection or screening results. In some embodiments, the prenatal care, prenatal clinical treatment, or other decision or action is carried out if the GA of the pregnant subject has been classified as lower than the predetermined GA cutpoint. In some embodiments, the prenatal care, prenatal clinical treatment, or other decision or action is carried out if the GA of the pregnant subject has been classified as higher than the predetermined GA cutpoint.

A. Classifying Gestational Age

The provided methods in some embodiments include using an immunoassay (e.g., any of the immunoassays described in Section II-C) to detect PAPP-A in a biological sample from a pregnant subject. In some embodiments, the immunoassay involves assessing the degree of a detectable signal indicative of the amount of one or more PAPP-A proteoforms in a test sample derived from the biological sample, e.g., a test sample prepared using any of the methods described in Section II-B. In some embodiments, the detectable signal is produced by a detectable label conjugated to the detection antibody. In some embodiments, the detectable signal is detected when the detection antibody is bound to the one or more PAPP-A proteoforms or in a complex containing the one or more PAPP-A proteoforms.

In some embodiments, the degree of the detectable signal from the test sample is compared to the degree of a detectable signal assessed from a reference PAPP-A sample using the immunoassay that is indicative of the concentration of PAPP-A in the reference PAPP-A sample. In some embodiments, the concentration of PAPP-A in the reference PAPP-A sample is a concentration that is associated with a predetermined GA cutpoint. In some embodiments, the concentration of PAPP-A in the reference PAPP-A sample is predetermined based on knowledge of the concentration of PAPP-A in samples from female subjects across a range of GAs. In some embodiments, the concentration of PAPP-A in the reference PAPP-A sample is predetermined based on samples from a plurality of subjects, wherein the samples have been previously assessed for the correlation or association of PAPP-A to GA as determined by other methods, such as ultrasound.

In some embodiments, the reference PAPP-A sample includes one or more proteoforms of PAPP-A. In some embodiments, the reference PAPP-A sample includes homodimeric PAPP-A. In some embodiments, the reference PAPP-A sample includes the heterotetrameric PAPP-A/proMBP complex. In some embodiments, the reference PAPP-A sample includes homodimeric PAPP-A and the heterotetrameric PAPP-A/proMBP complex. In some embodiments, the reference PAPP-A sample consists essentially of the heterotetrameric PAPP-A/proMBP complex.

In some embodiments, the PAPP-A in the reference PAPP-A sample is detected using an immunoassay. In some embodiments, the immunoassay is any as described in Section II-C. In some embodiments, the immunoassay is the same immunoassay used to detect PAPP-A in the biological sample from the pregnant subject.

In some embodiments of the provided methods, the concentration of PAPP-A in the reference PAPP-A sample is for classifying GA based on a predetermined GA cutpoint. In some embodiments, the concentration of PAPP-A in the reference PAPP-A sample is a concentration that is associated with the predetermined GA cutpoint. In some embodiments, the GA of the pregnancy is classified as above the predetermined GA cutpoint if the degree of the detectable signal from the test sample from the pregnant subject is greater than or equal to the degree of the detectable signal from the reference PAPP-A sample. In some embodiments, the GA of the pregnancy is classified as below the predetermined GA cutpoint if the degree of the detectable signal assessed from the test sample from the pregnant subject is less than the degree of the detectable signal assessed from the reference PAPP-A sample.

In some embodiments, the immunoassay further includes measuring the concentration of PAPP-A in the biological sample by comparison of the degree of the detectable signal from the test sample to a standard curve. In some embodiments, the provided methods involve comparing the concentration of PAPP-A in the biological sample to a predetermined concentration associated with the predetermined GA cutpoint. In some embodiments, the predetermined concentration is determined using the same immunoassay. In some embodiments, the provided methods involve comparing the concentration of PAPP-A in the biological sample to the concentration of PAPP-A in a reference PAPP-A sample. In some embodiments, the GA of the pregnancy is classified as above the predetermined GA cutpoint if the concentration of PAPP-A in the biological sample from the pregnant subject is greater than or equal to the predetermined concentration. In some embodiments, the GA of the pregnancy is classified as below the predetermined GA cutpoint if the concentration of PAPP-A in the biological sample from the pregnant subject is less than the predetermined concentration.

In some embodiments of the provided methods, the concentration of PAPP-A in the reference PAPP-A sample is associated with a predetermined GA cutpoint that is a timepoint between or between about 5 weeks and 40 weeks. In some embodiments, the predetermined GA cutpoint is a timepoint between at or about 5 weeks and at or about 35 weeks. In some embodiments, the predetermined GA cutpoint is a timepoint between at or about 5 weeks and at or about 30 weeks. In some embodiments, the predetermined GA cutpoint is a timepoint between at or about 5 weeks and at or about 25 weeks. In some embodiments, the predetermined GA cutpoint is a timepoint between at or about 5 weeks and at or about 20 weeks. In some embodiments, the predetermined GA cutpoint is a timepoint between at or about 5 weeks and at or about 19 weeks. In some embodiments, the predetermined GA cutpoint is a timepoint between at or about 5 weeks and at or about 18 weeks. In some embodiments, the predetermined GA cutpoint is a timepoint between at or about 5 weeks and at or about 17 weeks. In some embodiments, the predetermined GA cutpoint is a timepoint between at or about 5 weeks and at or about 16 weeks. In some embodiments, the predetermined GA cutpoint is a timepoint between at or about 5 weeks and at or about 15 weeks (105 days). In some embodiments, the predetermined GA cutpoint is a timepoint between at or about 5 weeks and at or about 14 weeks. In some embodiments, the predetermined GA cutpoint is a timepoint between at or about 5 weeks and at or about 13 weeks. In some embodiments, the predetermined GA cutpoint is a timepoint between at or about 5 weeks and at or about 12 weeks. In some embodiments, the predetermined GA cutpoint is a timepoint between at or about 5 weeks and at or about 11 weeks. In some embodiments, the predetermined GA cutpoint is a timepoint between at or about 5 weeks and at or about 10 weeks. In some embodiments, the predetermined GA cutpoint is a timepoint between at or about 5 weeks and at or about 9 weeks. In particular embodiments, the predetermined GA cutpoint is at or about 8 weeks. In particular embodiments, the predetermined GA cutpoint is at or about 9 weeks. In particular embodiments, the predetermined GA cutpoint is at or about 10 weeks. In other embodiments, the predetermined GA cutpoint is at or about 11 weeks. In further embodiments, the predetermined GA cutpoint is at or about 12 weeks. In particular embodiments, the predetermined GA cutpoint is at or about 13 weeks. In particular embodiments, the predetermined GA cutpoint is at or about 14 weeks. In particular embodiments, the predetermined GA cutpoint is at or about 15 weeks (105 days). In particular embodiments, the predetermined GA cutpoint is at or about 16 weeks. In particular embodiments, the predetermined GA cutpoint is at or about 17 weeks. In particular embodiments, the predetermined GA cutpoint is at or about 18 weeks. In particular embodiments, the predetermined GA cutpoint is at or about 19 weeks. In particular embodiments, the predetermined GA cutpoint is at or about 20 weeks.

In embodiments of the provided methods, the predetermined GA cutpoint is a timepoint between at or about 56 days and at or about 280 days. In some embodiments, the GA cutpoint is a timepoint between at or about 56 days and at or about 140 days. In some embodiments, the predetermined GA cutpoint is a timepoint between at or about 56 days and at or about 112 days. In some embodiments, the predetermined GA cutpoint is a timepoint between at or about 63 days and at or about 105 days. In provided embodiments, the predetermined GA cutpoint is at or about 64 days, at or about 66 days, at or about 68 days, at or about 70 days, at or about 72 days, at or about 74 days, at or about 76 days, at or about 78 days, at or about 80 days, at or about 82 days, at or about 84 days, at or about 86 days, at or about 88 days, at or about 90 days, at or about 92 days, at or about 94 days, at or about 96 days, at or about 98 days, at or about 100 days, at or about 102 days, at or about 104 days, at or about 106 days, at or about 108 days or at or about 110 days, at or about 120 days, at or about 130 days, at or about 140 days, at or about 160 days, at or about 180 days, at or about 200 days, at or about 220 days, at or about 240 days or at or about 280 days, or is any time point between any of the foregoing.

In some embodiments, the predetermined GA cutpoint is a timepoint between or between about 56 days and 84 days. The ability to classify GA as above or below time points in this range can be useful in informing a variety of prenatal care or clinical treatment decisions, including concerning the dose of misoprostol used in a medical abortion, whether a medical or surgical abortion would be feasible for a given patient, the types of clinical support that would be needed at that GA, what pre-abortion tests would be required (e.g., ultrasound or RH testing), and whether a medical or surgical abortion would be desired at all. In some embodiments, the predetermined GA cutpoint is a timepoint between or between about 63 days and 77 days. In some embodiments, the predetermined GA cutpoint is or is about 56 days. In some embodiments, the predetermined GA cutpoint is or is about 63 days. In some embodiments, the predetermined GA cutpoint is or is about 70 days. In some embodiments, the predetermined GA cutpoint is or is about 77 days. In some embodiments, the predetermined GA cutpoint is or is about 84 days.

In some embodiments, the predetermined GA cutpoint is a timepoint between or between about 98 days and 112 days. The ability to classify GA as above or below time points in this range can be useful in informing a variety of prenatal care or clinical treatment decisions, including in determining if a patient wants to pursue abortion at all and, if so, the level of clinical support, provider skill (e.g., as it relates to performing surgical abortion), or counseling regarding risks of complications that would be required. In some embodiments, the predetermined GA cutpoint is or is about 98 days. In some embodiments, the predetermined GA cutpoint is or is about 105 days. In some embodiments, the predetermined GA cutpoint is or is about 112 days.

In some embodiments, the predetermined GA cutpoint is or is about 168 days. As many US states prohibit abortion beyond this timepoint, the ability to classify GA as above or below this GA cutpoint can be useful in assessing the possibility of receiving an abortion.

In some embodiments, the concentration of PAPP-A in the reference PAPP-A sample is predetermined based on PAPP-A amounts or concentrations in samples from a population of pregnant subjects known to have pregnancies of 5-40 weeks gestation, for instance as determined by ultrasound. In some embodiments, the population is chosen using quotas to ensure that PAPP-A amounts or concentrations across the full duration of pregnancy are estimated. In some embodiments, the population is oversampled at particular GAs of interest. In some embodiments, the population includes an oversampling of pregnant subjects with GAs within two weeks below and two weeks above a predetermined GA cutpoint. In some embodiments, the predetermined GA cutpoint is 10 weeks (70 days), and the population includes an oversampling of pregnant subjects with GAs between or between about 8 and 10 weeks.

Any of a variety of methods can be used to set or identify a concentration of PAPP-A in the reference PAPP-A sample with which there is good predictive value for a particular GA cutpoint. Exemplary concentrations of PAPP-A in reference PAPP-A samples that are associated with certain predetermined GA cutpoints are described in Section III-A-1. In some embodiments, the concentration of PAPP-A in the reference PAPP-A sample is chosen based on one or more evaluation metrics (e.g., accuracy, sensitivity, specificity, and/or AUC). In some embodiments, the concentration of PAPP-A in the reference PAPP-A sample is chosen based on two or more evaluation metrics.

In some embodiments, the concentration of PAPP-A in the reference PAPP-A sample is chosen to maximize the accuracy of the test (i.e., chosen to maximize the number of correctly classified GAs). In some embodiments, the concentration of PAPP-A in the reference PAPP-A sample is chosen to maximize the sensitivity of the test (i.e., chosen to maximize the number of positive test results for pregnant subjects with GAs higher than the predetermined GA cutpoint) or to maximize the positive predictive value of the test (i.e., chosen to minimize the number of positive test results for pregnant subjects with GAs lower than the predetermined GA cutpoint). In some embodiments, the concentration of PAPP-A in the reference PAPP-A sample is chosen to maximize the specificity of the test (i.e., chosen to maximize the number of negative test results for pregnant subjects with GAs lower than the predetermined GA cutpoint) or to maximize the negative predictive value of the test (i.e., chosen to minimize the number of negative test results for pregnant subjects with GAs higher than the predetermined GA cutpoint).

In some embodiments, the concentration of PAPP-A in the reference PAPP-A sample is chosen to maximize sensitivity in classifying GAs as above or below the predetermined GA cutpoint. In some embodiments, the concentration of PAPP-A in the reference PAPP-A sample is chosen to maximize sensitivity in classifying GAs as above or below the predetermined GA cutpoint. In some cases, the concentration of PAPP-A in the reference PAPP-A sample is chosen to maximize the Youden Index (i.e., specificity+sensitivity−1). In some embodiments, the concentration of PAPP-A in the reference PAPP-A sample is chosen to maximize specificity and sensitivity in classifying GAs as above or below the predetermined GA cutpoint. In some of any such embodiments, the predetermined GA cutpoint is 10 weeks (70 days).

In some embodiments, the concentration of PAPP-A in the reference PAPP-A sample provides for a certain level of sensitivity in classifying GAs as above or below the predetermined GA cutpoint (e.g., a GA cutpoint of 70 days). In some embodiments, the classifying is performed with greater than or greater than about 50% sensitivity in classifying GAs. In some embodiments, the classifying is performed with greater than or greater than about 60% sensitivity in classifying GAs. In some embodiments, the classifying is performed with greater than or greater than about 70% sensitivity in classifying GAs. In some embodiments, the classifying is performed with greater than or greater than about 75% sensitivity in classifying GAs. In some embodiments, the classifying is performed with greater than or greater than about 80% sensitivity in classifying GAs. In some embodiments, the classifying is performed with greater than or greater than about 85% sensitivity in classifying GAs. In some embodiments, the classifying is performed with greater than or greater than about 90% sensitivity in classifying GAs.

In some embodiments, the concentration of PAPP-A in the reference PAPP-A sample provides for a certain level of specificity in classifying GAs as above or below the predetermined GA cutpoint (e.g., a GA cutpoint of 70 days). In some embodiments, the classifying is performed with greater than or greater than about 50% specificity in classifying GAs. In some embodiments, the classifying is performed with greater than or greater than about 60% specificity in classifying GAs. In some embodiments, the classifying is performed with greater than or greater than about 70% specificity in classifying GAs. In some embodiments, the classifying is performed with greater than or greater than about 75% specificity in classifying GAs. In some embodiments, the classifying is performed with greater than or greater than about 80% specificity in classifying GAs. In some embodiments, the classifying is performed with greater than or greater than about 85% specificity in classifying GAs. In some embodiments, the classifying is performed with greater than or greater than about 90% specificity in classifying GAs.

In some embodiments, the concentration of PAPP-A in the reference PAPP-A sample provides for a certain level of sensitivity and specificity in classifying GAs as above or below the predetermined GA cutpoint (e.g., a GA cutpoint of 70 days). In some embodiments, the classifying is performed with greater than or greater than about 50% sensitivity and specificity in classifying GAs. In some embodiments, the classifying is performed with greater than or greater than about 60% sensitivity and specificity in classifying GAs. In some embodiments, the classifying is performed with greater than or greater than about 70% sensitivity and specificity in classifying GAs. In some embodiments, the classifying is performed with greater than or greater than about 75% sensitivity and specificity in classifying GAs. In some embodiments, the classifying is performed with greater than or greater than about 80% sensitivity and specificity in classifying GAs. In some embodiments, the classifying is performed with greater than or greater than about 85% sensitivity and specificity in classifying GAs. In some embodiments, the classifying is performed with greater than or greater than about 90% sensitivity and specificity in classifying GAs.

In some embodiments, the concentration of PAPP-A in the reference PAPP-A sample provides for a certain AUC level in classifying GAs as above or below the predetermined GA cutpoint (e.g., a GA cutpoint of 70 days). In some embodiments, the AUC is greater than or greater than about 0.5. In some embodiments, the AUC is greater than or greater than about 0.6. In some embodiments, the AUC is greater than or greater than about 0.7. In some embodiments, the AUC is greater than or greater than about 0.75. In some embodiments, the AUC is greater than or greater than about 0.8. In some embodiments, the AUC is greater than or greater than about 0.85. In some embodiments, the AUC is greater than or greater than about 0.9. In some embodiments, the AUC is greater than or greater than about 0.95.

In some embodiments, the GA of the pregnancy is classified by comparing the degree of the detectable signal from the test sample with the degrees of detectable signals from two or more reference PAPP-A samples. In some embodiments, the detectable signals from the two or more reference PAPP-A samples are detected using any of the methods provided herein. In some embodiments, the provided methods further include detecting PAPP-A in each of the two or more reference PAPP-A samples using any of the methods provided herein.

In some embodiments, the first reference PAPP-A sample is for a first predetermined GA cutpoint. In some embodiments, the first reference PAPP-A sample and the first predetermined GA cutpoint are any as described herein. In some embodiments, the second reference PAPP-A sample is for a second predetermined GA cutpoint that is different from the first predetermined GA cutpoint, e.g., a second predetermined GA cutpoint that is higher than the first predetermined GA cutpoint. In some embodiments, the second reference PAPP-A sample and the second predetermined GA cutpoint are any as described herein. Exemplary concentrations of PAPP-A in first and second reference PAPP-A samples that correspond to certain first and second predetermined GA cutpoints are described in Section III-A-1.

In some embodiments, the first predetermined GA cutpoint and the second predetermined GA cutpoint are each a timepoint between or between about 56 and 84 days. In some embodiments, the first predetermined GA cutpoint and the second predetermined GA cutpoint are each a timepoint between or between about 63 and 77 days. In some embodiments, the first predetermined GA cutpoint and the second predetermined GA cutpoint are independently selected from or from about 63 days, 70 days, and 77 days. In some embodiments, the first predetermined GA cutpoint is or is about 63 days, and the second predetermined GA cutpoint is or is about 77 days.

In some embodiments, the first predetermined GA cutpoint and the second predetermined GA cutpoint are each a timepoint between or between about 98 and 112 days. In some embodiments, the first predetermined GA cutpoint and the second predetermined GA cutpoint are independently selected from or from about 98 days, 105 days, and 112 days. In some embodiments, the first predetermined GA cutpoint is or is about 98 days, and the second predetermined GA cutpoint is or is about 112 days.

In some embodiments, the first predetermined GA cutpoint is a timepoint between or between about 56 and 84 days, and the second predetermined GA cutpoint is a timepoint between or between about 98 and 112 days. In some embodiments, the first predetermined GA cutpoint is a timepoint between or between about 63 and 77 days, and the second predetermined GA cutpoint is a timepoint between or between about 98 and 112 days. In some embodiments, the first predetermined GA cutpoint is or is about 70 days, and the second predetermined GA cutpoint is or is about 105 days.

I. Exemplary PAPP-A Concentrations in Reference PAPP-A Samples

In some embodiments, the predetermined GA cutpoint is at or about 56 days or 8 weeks. In some of any such embodiments, the concentration of PAPP-A in the reference PAPP-A sample is between or between about 1 ng/mL and 70 ng/mL, such as between or between about 1 ng/mL and 65 ng/mL, 1 ng/mL and 60 ng/mL, 1 ng/mL and 55 ng/mL, 2 ng/mL and 70 ng/mL, 2 ng/mL and 65 ng/mL, 2 ng/mL and 60 ng/mL, 2 ng/mL and 55 ng/mL, 4 ng/mL and 70 ng/mL, 4 ng/mL and 65 ng/mL, 4 ng/mL and 60 ng/mL, 4 ng/mL and 55 ng/mL, 6 ng/mL and 70 ng/mL, 6 ng/mL and 65 ng/mL, 6 ng/mL and 60 ng/mL, 6 ng/mL and 55 ng/mL, 8 ng/mL and 70 ng/mL, 8 ng/mL and 65 ng/mL, 8 ng/mL and 60 ng/mL, or 8 ng/mL and 55 ng/mL, each inclusive. In some embodiments, the concentration of PAPP-A in the reference PAPP-A sample is between or between about 5 ng/mL and 55 ng/mL. In some embodiments, the concentration of PAPP-A in the reference PAPP-A sample is between or between about 1 ng/mL and 20 ng/mL. In some embodiments, the concentration of PAPP-A in the reference PAPP-A sample is between or between about 5 ng/mL and 15 ng/mL. In some embodiments, the concentration of PAPP-A in the reference PAPP-A sample is or is about 8.197 ng/mL. In some embodiments, the concentration of PAPP-A in the reference PAPP-A sample is between or between about 40 ng/mL and 60 ng/mL. In some embodiments, the concentration of PAPP-A in the reference PAPP-A sample is between or between about 45 ng/mL and 60 ng/mL. In some embodiments, the concentration of PAPP-A in the reference PAPP-A sample is between or between about 45 ng/mL and 55 ng/mL. In some embodiments, the concentration of PAPP-A in the reference PAPP-A sample is or is about 52.346 ng/mL.

In some embodiments, the predetermined GA cutpoint is at or about 63 days or 9 weeks. In some embodiments, the concentration of PAPP-A in the reference PAPP-A sample is between or between about 25 ng/mL and 150 ng/mL, such as between or between about 25 ng/mL and 145 ng/mL, 25 ng/mL and 140 ng/mL, 25 ng/mL and 135 ng/mL, 25 ng/mL and 130 ng/mL, 25 ng/mL and 125 ng/mL, 30 ng/mL and 150 ng/mL, 30 ng/mL and 145 ng/mL, 30 ng/mL and 140 ng/mL, 30 ng/mL and 135 ng/mL, 30 ng/mL and 130 ng/mL, 30 ng/mL and 125 ng/mL, 35 ng/mL and 150 ng/mL, 35 ng/mL and 145 ng/mL, 35 ng/mL and 140 ng/mL, 35 ng/mL and 135 ng/mL, 35 ng/mL and 130 ng/mL, 35 ng/mL and 125 ng/mL, 40 ng/mL and 150 ng/mL, 40 ng/mL and 145 ng/mL, 40 ng/mL and 140 ng/mL, 40 ng/mL and 135 ng/mL, 40 ng/mL and 130 ng/mL, 40 ng/mL and 125 ng/mL, 45 ng/mL and 150 ng/mL, 45 ng/mL and 145 ng/mL, 45 ng/mL and 140 ng/mL, 45 ng/mL and 135 ng/mL, 45 ng/mL and 130 ng/mL, 45 ng/mL and 125 ng/mL, 50 ng/mL and 150 ng/mL, 50 ng/mL and 145 ng/mL, 50 ng/mL and 140 ng/mL, 50 ng/mL and 135 ng/mL, 50 ng/mL and 130 ng/mL, or 50 ng/mL and 125 ng/mL, each inclusive. In some embodiments, the concentration of PAPP-A in the reference PAPP-A sample is between or between about 40 ng/mL and 130 ng/mL. In some embodiments, the concentration of PAPP-A in the reference PAPP-A sample is between or between about 40 ng/mL and 60 ng/mL. In some embodiments, the concentration of PAPP-A in the reference PAPP-A sample is between or between about 45 ng/mL and 55 ng/mL. In some embodiments, the concentration of PAPP-A in the reference PAPP-A sample is or is about 52.346 ng/mL. In some embodiments, the concentration of PAPP-A in the reference PAPP-A sample is between or between about 100 ng/mL and 140 ng/mL. In some embodiments, the concentration of PAPP-A in the reference PAPP-A sample is between or between about 110 ng/mL and 130 ng/mL. In some embodiments, the concentration of PAPP-A in the reference PAPP-A sample is between or between about 115 ng/mL and 125 ng/mL. In some embodiments, the concentration of PAPP-A in the reference PAPP-A sample is or is about 123.733 ng/mL.

In some embodiments, the predetermined GA cutpoint is at or about 70 days or 10 weeks. In some embodiments, the concentration of PAPP-A in the reference PAPP-A sample is between or between about 60 ng/mL and 200 ng/mL, such as between or between about 60 ng/mL and 200 ng/mL, 60 ng/mL and 195 ng/mL, 60 ng/mL and 190 ng/mL, 60 ng/mL and 185 ng/mL, 60 ng/mL and 180 ng/mL, 65 ng/mL and 200 ng/mL, 65 ng/mL and 195 ng/mL, 65 ng/mL and 190 ng/mL, 65 ng/mL and 185 ng/mL, 65 ng/mL and 180 ng/mL, 70 ng/mL and 200 ng/mL, 70 ng/mL and 195 ng/mL, 70 ng/mL and 190 ng/mL, 70 ng/mL and 185 ng/mL, 70 ng/mL and 180 ng/mL, 75 ng/mL and 200 ng/mL, 75 ng/mL and 195 ng/mL, 75 ng/mL and 190 ng/mL, 75 ng/mL and 185 ng/mL, or 75 ng/mL and 180 ng/mL, each inclusive. In some embodiments, the concentration of PAPP-A in the reference PAPP-A sample is between or between about 70 ng/mL and 185 ng/mL. In some embodiments, the concentration of PAPP-A in the reference PAPP-A sample is between or between about 70 ng/mL and 90 ng/mL. In some embodiments, the concentration of PAPP-A in the reference PAPP-A sample is between or between about 75 ng/mL and 85 ng/mL. In some embodiments, the concentration of PAPP-A in the reference PAPP-A sample is or is about 82.816 ng/mL. In some embodiments, the concentration of PAPP-A in the reference PAPP-A sample is between or between about 160 ng/mL and 200 ng/mL. In some embodiments, the concentration of PAPP-A in the reference PAPP-A sample is between or between about 170 ng/mL and 190 ng/mL. In some embodiments, the concentration of PAPP-A in the reference PAPP-A sample is between or between about 175 ng/mL and 185 ng/mL. In some embodiments, the concentration of PAPP-A in the reference PAPP-A sample is or is about 180.697 ng/mL.

In some embodiments, the predetermined GA cutpoint is at or about 70 days or 10 weeks. In some embodiments, the concentration of PAPP-A in the reference PAPP-A sample is between or between about 20 ng/mL and 200 ng/mL. In some embodiments, the concentration of PAPP-A in the reference PAPP-A sample is between or between about 20 ng/mL and 190 ng/mL. In some embodiments, the concentration of PAPP-A in the reference PAPP-A sample is between or between about 20 ng/mL and 180 ng/mL. In some embodiments, the concentration of PAPP-A in the reference PAPP-A sample is between or between about 20 ng/mL and 170 ng/mL. In some embodiments, the concentration of PAPP-A in the reference PAPP-A sample is between or between about 20 ng/mL and 160 ng/mL. In some embodiments, the concentration of PAPP-A in the reference PAPP-A sample is between or between about 20 ng/mL and 150 ng/mL. In some embodiments, the concentration of PAPP-A in the reference PAPP-A sample is between or between about 20 ng/mL and 140 ng/mL. In some embodiments, the concentration of PAPP-A in the reference PAPP-A sample is between or between about 30 ng/mL and 200 ng/mL. In some embodiments, the concentration of PAPP-A in the reference PAPP-A sample is between or between about 30 ng/mL and 190 ng/mL. In some embodiments, the concentration of PAPP-A in the reference PAPP-A sample is between or between about 30 ng/mL and 180 ng/mL. In some embodiments, the concentration of PAPP-A in the reference PAPP-A sample is between or between about 30 ng/mL and 170 ng/mL. In some embodiments, the concentration of PAPP-A in the reference PAPP-A sample is between or between about 30 ng/mL and 160 ng/mL. In some embodiments, the concentration of PAPP-A in the reference PAPP-A sample is between or between about 30 ng/mL and 150 ng/mL. In some embodiments, the concentration of PAPP-A in the reference PAPP-A sample is between or between about 30 ng/mL and 140 ng/mL. In some embodiments, the concentration of PAPP-A in the reference PAPP-A sample is between or between about 40 ng/mL and 200 ng/mL. In some embodiments, the concentration of PAPP-A in the reference PAPP-A sample is between or between about 40 ng/mL and 190 ng/mL. In some embodiments, the concentration of PAPP-A in the reference PAPP-A sample is between or between about 40 ng/mL and 180 ng/mL. In some embodiments, the concentration of PAPP-A in the reference PAPP-A sample is between or between about 40 ng/mL and 170 ng/mL. In some embodiments, the concentration of PAPP-A in the reference PAPP-A sample is between or between about 40 ng/mL and 160 ng/mL. In some embodiments, the concentration of PAPP-A in the reference PAPP-A sample is between or between about 40 ng/mL and 150 ng/mL. In some embodiments, the concentration of PAPP-A in the reference PAPP-A sample is between or between about 40 ng/mL and 140 ng/mL.

In some embodiments, the predetermined GA cutpoint is at or about 70 days or 10 weeks. In some embodiments, the concentration of PAPP-A in the reference PAPP-A sample is between or between about 20 ng/mL and 60 ng/mL. In some embodiments, the concentration of PAPP-A in the reference PAPP-A sample is between or between about 30 ng/mL and 50 ng/mL. In some embodiments, the concentration of PAPP-A in the reference PAPP-A sample is between or between about 35 ng/mL and 45 ng/mL. In some embodiments, the concentration of PAPP-A in the reference PAPP-A sample is or is about 44.153 ng/mL.

In some embodiments, the concentration of PAPP-A in the reference PAPP-A sample is between or between about 110 ng/mL and 150 ng/mL. In some embodiments, the concentration of PAPP-A in the reference PAPP-A sample is between or between about 120 ng/mL and 140 ng/mL. In some embodiments, the concentration of PAPP-A in the reference PAPP-A sample is between or between about 125 ng/mL and 135 ng/mL. In some embodiments, the concentration of PAPP-A in the reference PAPP-A sample is or is about 133.2096 ng/mL.

In some embodiments, the predetermined GA cutpoint is at or about 77 days or 11 weeks. In some of any such embodiments, the concentration of PAPP-A in the reference PAPP-A sample is between or between about 80 ng/mL and 220 ng/mL, such as between or between about 80 ng/mL and 220 ng/mL, 80 ng/mL and 215 ng/mL, 80 ng/mL and 210 ng/mL, 85 ng/mL and 220 ng/mL, 85 ng/mL and 215 ng/mL, 85 ng/mL and 210 ng/mL, 90 ng/mL and 220 ng/mL, 90 ng/mL and 215 ng/mL, 90 ng/mL and 210 ng/mL, 95 ng/mL and 220 ng/mL, 95 ng/mL and 215 ng/mL, or 95 ng/mL and 210 ng/mL, each inclusive. In some embodiments, the concentration of PAPP-A in the reference PAPP-A sample is between or between about 90 ng/mL and 215 ng/mL. In some embodiments, the concentration of PAPP-A in the reference PAPP-A sample is between or between about 90 ng/mL and 110 ng/mL. In some embodiments, the concentration of PAPP-A in the reference PAPP-A sample is between or between about 95 ng/mL and 105 ng/mL. In some embodiments, the concentration of PAPP-A in the reference PAPP-A sample is or is about 101.351 ng/mL. In some embodiments, the concentration of PAPP-A in the reference PAPP-A sample is between or between about 195 ng/mL and 215 ng/mL. In some embodiments, the concentration of PAPP-A in the reference PAPP-A sample is between or between about 200 ng/mL and 210 ng/mL. In some embodiments, the concentration of PAPP-A in the reference PAPP-A sample is or is about 206.252 ng/mL.

In some embodiments, the predetermined GA cutpoint is at or about 84 days or 12 weeks. In some of any such embodiments, the concentration of PAPP-A in the reference PAPP-A sample is between or between about 160 ng/mL and 480 ng/mL, such as between or between about 160 ng/mL and 480 ng/mL, 160 ng/mL and 475 ng/mL, 160 ng/mL and 470 ng/mL, 160 ng/mL and 465 ng/mL, 165 ng/mL and 480 ng/mL, 165 ng/mL and 475 ng/mL, 165 ng/mL and 470 ng/mL, 165 ng/mL and 465 ng/mL, 170 ng/mL and 480 ng/mL, 170 ng/mL and 475 ng/mL, 170 ng/mL and 470 ng/mL, 170 ng/mL and 465 ng/mL, 175 ng/mL and 480 ng/mL, 175 ng/mL and 475 ng/mL, 175 ng/mL and 470 ng/mL, or 175 ng/mL and 465 ng/mL, each inclusive. In some embodiments, the concentration of PAPP-A in the reference PAPP-A sample is between or between about 170 ng/mL and 470 ng/mL. In some embodiments, the concentration of PAPP-A in the reference PAPP-A sample is between or between about 170 ng/mL and 190 ng/mL. In some embodiments, the concentration of PAPP-A in the reference PAPP-A sample is between or between about 175 ng/mL and 185 ng/mL. In some embodiments, the concentration of PAPP-A in the reference PAPP-A sample is or is about 182.514 ng/mL. In some embodiments, the concentration of PAPP-A in the reference PAPP-A sample is between or between about 455 ng/mL and 475 ng/mL. In some embodiments, the concentration of PAPP-A in the reference PAPP-A sample is between or between about 460 ng/mL and 470 ng/mL. In some embodiments, the concentration of PAPP-A in the reference PAPP-A sample is or is about 463.890 ng/mL.

In some embodiments, the predetermined GA cutpoint is at or about 98 days or 14 weeks. In some embodiments, the concentration of PAPP-A in the reference PAPP-A sample is between or between about 550 ng/mL and 1240 ng/mL, such as between or between about 550 ng/mL and 1235 ng/mL, 550 ng/mL and 1230 ng/mL, 550 ng/mL and 1225 ng/mL, 550 ng/mL and 1220 ng/mL, 555 ng/mL and 1235 ng/mL, 555 ng/mL and 1230 ng/mL, 555 ng/mL and 1225 ng/mL, 555 ng/mL and 1220 ng/mL, 560 ng/mL and 1235 ng/mL, 560 ng/mL and 1230 ng/mL, 560 ng/mL and 1225 ng/mL, 560 ng/mL and 1220 ng/mL, 565 ng/mL and 1235 ng/mL, 565 ng/mL and 1230 ng/mL, 565 ng/mL and 1225 ng/mL, 565 ng/mL and 1220 ng/mL, 570 ng/mL and 1235 ng/mL, 570 ng/mL and 1230 ng/mL, 570 ng/mL and 1225 ng/mL, or 570 ng/mL and 1220 ng/mL, each inclusive. In some embodiments, the concentration of PAPP-A in the reference PAPP-A sample is between or between about 565 ng/mL and 1230 ng/mL. In some embodiments, the concentration of PAPP-A in the reference PAPP-A sample is between or between about 565 ng/mL and 585 ng/mL. In some embodiments, the concentration of PAPP-A in the reference PAPP-A sample is between or between about 570 ng/mL and 580 ng/mL. In some embodiments, the concentration of PAPP-A in the reference PAPP-A sample is or is about 574.025 ng/mL. In some embodiments, the concentration of PAPP-A in the reference PAPP-A sample is between or between about 1205 ng/mL and 1225 ng/mL. In some embodiments, the concentration of PAPP-A in the reference PAPP-A sample is between or between about 1210 ng/mL and 1220 ng/mL. In some embodiments, the concentration of PAPP-A in the reference PAPP-A sample is or is about 1215.391 ng/mL.

In some embodiments, the predetermined GA cutpoint is at or about 105 days or 15 weeks. In some embodiments, the concentration of PAPP-A in the reference PAPP-A sample is between or between about 1440 ng/mL and 1490 ng/mL, such as between or between about 1440 ng/mL and 1490 ng/mL, 1440 ng/mL and 1485 ng/mL, 1440 ng/mL and 1480 ng/mL, 1440 ng/mL and 1475 ng/mL, 1440 ng/mL and 1470 ng/mL, 1445 ng/mL and 1490 ng/mL, 1445 ng/mL and 1485 ng/mL, 1445 ng/mL and 1480 ng/mL, 1445 ng/mL and 1475 ng/mL, 1445 ng/mL and 1470 ng/mL, 1450 ng/mL and 1490 ng/mL, 1450 ng/mL and 1485 ng/mL, 1450 ng/mL and 1480 ng/mL, 1450 ng/mL and 1475 ng/mL, 1450 ng/mL and 1470 ng/mL, 1455 ng/mL and 1490 ng/mL, 1455 ng/mL and 1485 ng/mL, 1455 ng/mL and 1480 ng/mL, 1455 ng/mL and 1475 ng/mL, 1455 ng/mL and 1470 ng/mL, 1460 ng/mL and 1490 ng/mL, 1460 ng/mL and 1485 ng/mL, 1460 ng/mL and 1480 ng/mL, 1460 ng/mL and 1475 ng/mL, or 1460 ng/mL and 1470 ng/mL, each inclusive. In some embodiments, the concentration of PAPP-A in the reference PAPP-A sample is between or between about 1450 ng/mL and 1475 ng/mL. In some embodiments, the concentration of PAPP-A in the reference PAPP-A sample is between or between about 1455 ng/mL and 1475 ng/mL. In some embodiments, the concentration of PAPP-A in the reference PAPP-A sample is between or between about 1460 ng/mL and 1470 ng/mL. In some embodiments, the concentration of PAPP-A in the reference PAPP-A sample is or is about 1465.904 ng/mL.

In some embodiments, the predetermined GA cutpoint is at or about 112 days or 16 weeks. In some embodiments, the concentration of PAPP-A in the reference PAPP-A sample is between or between about 1440 ng/mL and 1490 ng/mL, such as between or between about 1440 ng/mL and 1490 ng/mL, 1440 ng/mL and 1485 ng/mL, 1440 ng/mL and 1480 ng/mL, 1440 ng/mL and 1475 ng/mL, 1440 ng/mL and 1470 ng/mL, 1445 ng/mL and 1490 ng/mL, 1445 ng/mL and 1485 ng/mL, 1445 ng/mL and 1480 ng/mL, 1445 ng/mL and 1475 ng/mL, 1445 ng/mL and 1470 ng/mL, 1450 ng/mL and 1490 ng/mL, 1450 ng/mL and 1485 ng/mL, 1450 ng/mL and 1480 ng/mL, 1450 ng/mL and 1475 ng/mL, 1450 ng/mL and 1470 ng/mL, 1455 ng/mL and 1490 ng/mL, 1455 ng/mL and 1485 ng/mL, 1455 ng/mL and 1480 ng/mL, 1455 ng/mL and 1475 ng/mL, 1455 ng/mL and 1470 ng/mL, 1460 ng/mL and 1490 ng/mL, 1460 ng/mL and 1485 ng/mL, 1460 ng/mL and 1480 ng/mL, 1460 ng/mL and 1475 ng/mL, or 1460 ng/mL and 1470 ng/mL, each inclusive. In some embodiments, the concentration of PAPP-A in the reference PAPP-A sample is between or between about 1450 ng/mL and 1475 ng/mL. In some embodiments, the concentration of PAPP-A in the reference PAPP-A sample is between or between about 1455 ng/mL and 1475 ng/mL. In some embodiments, the concentration of PAPP-A in the reference PAPP-A sample is between or between about 1460 ng/mL and 1470 ng/mL. In some embodiments, the concentration of PAPP-A in the reference PAPP-A sample is or is about 1465.904 ng/mL.

In some embodiments, the predetermined GA cutpoint is at or about 168 days or 24 weeks. In some embodiments, the concentration of PAPP-A in the reference PAPP-A sample is between or between about 3500 ng/mL and 4500 ng/mL, such as between or between about 3500 ng/mL and 4400 ng/mL, 3500 ng/mL and 4300 ng/mL, 3500 ng/mL and 4200 ng/mL, 3600 ng/mL and 4400 ng/mL, 3600 ng/mL and 4300 ng/mL, 3600 ng/mL and 4200 ng/mL, 3700 ng/mL and 4400 ng/mL, 3700 ng/mL and 4300 ng/mL, 3700 ng/mL and 4200 ng/mL, 3800 ng/mL and 4400 ng/mL, 3800 ng/mL and 4300 ng/mL, 3800 ng/mL and 4200 ng/mL, 3900 ng/mL and 4400 ng/mL, 3900 ng/mL and 4300 ng/mL, 3900 ng/mL and 4200 ng/mL, 4000 ng/mL and 4400 ng/mL, 4000 ng/mL and 4300 ng/mL, or 4000 ng/mL and 4200 ng/mL, each inclusive. In some embodiments, the concentration of PAPP-A in the reference PAPP-A sample is between or between about 4000 ng/mL and 4200 ng/mL. In some embodiments, the concentration of PAPP-A in the reference PAPP-A sample is between or between about 4050 ng/mL and 4150 ng/mL. In some embodiments, the concentration of PAPP-A in the reference PAPP-A sample is between or between about 4100 ng/mL and 4150 ng/mL. In some embodiments, the concentration of PAPP-A in the reference PAPP-A sample is or is about 4121.925 ng/mL.

B. Determining Gestational Age

In some embodiments, the provided methods include determining the GA of a pregnancy by detecting PAPP-A in a biological sample, e.g., a whole blood or serum sample, from a pregnant subject. Exemplary methods of detecting PAPP-A in a biological sample are described in Section II. In some embodiments, the determining of the GA is accomplished by assessing the degree of a detectable signal indicative of PAPP-A in the biological sample and providing the degree as input to a process that predicts the GA of the pregnancy based on the provided degree. In some embodiments, the determining of the GA is accomplished by providing the measured amount or concentration of PAPP-A in the biological sample as input to a process that uses PAPP-A amount or concentration as a predictor, e.g., a continuous predictor, of GA. In some embodiments, the process supplies a determined GA based on the degree of the detectable signal or the measured amount or concentration of PAPP-A provided as input. In some embodiments, the process is constructed based on knowledge of PAPP-A amounts or concentrations in samples from pregnant subjects across a range of GAs. For instance, the process in some embodiments is a model fit or trained using PAPP-A amounts or concentrations in samples from pregnant subjects across a range of GAs. In some embodiments, the process is constructed based also on the GAs of the pregnancies as determined by another clinical method, such as ultrasound.

In some embodiments, the process is a regression model that uses the amount or concentration of one or more proteins, e.g., PAPP-A and optionally other proteins, as continuous predictors for GA. Regression models are known in the art and include linear and non-linear models. In some embodiments, the process includes a plurality of regression models, for instance regression models using different protein amounts or concentrations or regression models fit or trained using specific GA windows or time periods. In some embodiments, the regression model is, or the plurality of regression models includes, a linear regression model, a piecewise linear model, a polynomial regression model, a Bayesian model, or any combination of any of the foregoing.

In some embodiments, the process is constructed based on PAPP-A amounts or concentrations from a plurality of pregnant subjects whose GAs fall within a particular range. In some embodiments, the range is between or between about 5 weeks and 40 weeks. In some embodiments, the range is between or between about 5 weeks and 33 weeks.

In some embodiments, the process is constructed based on PAPP-A amounts or concentrations in samples from a population of pregnant subjects known to have pregnancies of 5-40 weeks gestation, for instance as determined by ultrasound. In some embodiments, the population is any as described in Section III-A.

Any of a variety of methods can be used to construct and identify a process, for instance one or more regression models, with sufficient accuracy or performance in determining GA based on PAPP-A amount or concentration. Accuracy or performance of a regression model can be evaluated using one or more evaluation metrics. Such evaluation metrics are known in the art and include R-squared, Root Mean Squared Error (RMSE), Residual Standard Error (RSE), Mean Absolute Error (MAE), Adjusted R-squared, Akaike's Information Criteria (AIC), corrected AIC, Bayesian Information Criteria (BIC), and Mallows Cp. In some embodiments, high accuracy or performance for determining GAs within a particular GA range, e.g., between or between about 8 and 10 weeks, is prioritized.

In some embodiments, the process (e.g., one or more regression models) has high accuracy or performance in determining GA using PAPP-A amount or concentration in samples from pregnant subjects. In some embodiments, the GA determined by the process is or is on average within or within about 20% of the GA as determined by another clinical technique, for instance ultrasound. In some embodiments, the GA determined by the process is or is on average within or within about 18% of the GA as determined by another clinical technique. In some embodiments, the GA determined by the process is or is on average within or within about 16% of the GA as determined by another clinical technique. In some embodiments, the GA determined by the process is or is on average within or within about 14% of the GA as determined by another clinical technique. In some embodiments, the GA determined by the process is or is on average within or within about 12% of the GA as determined by another clinical technique. In some embodiments, the GA determined by the process is or is on average within or within about 10% of the GA as determined by another clinical technique. In some embodiments, the GA determined by the process is or is on average within or within about 9% of the GA as determined by another clinical technique. In some embodiments, the GA determined by the process is or is on average within or within about 8% of the GA as determined by another clinical technique. In some embodiments, the GA determined by the process is or is on average within or within about 7% of the GA as determined by another clinical technique. In some embodiments, the GA determined by the process is or is on average within or within about 6% of the GA as determined by another clinical technique. In some embodiments, the GA determined by the process is or is on average within or within about 5% of the GA as determined by another clinical technique. In some embodiments, the GA determined by the process is or is on average within or within about 4% of the GA as determined by another clinical technique. In some embodiments, the GA determined by the process is or is on average within or within about 3% of the GA as determined by another clinical technique. In some embodiments, the GA determined by the process is or is on average within or within about 2% of the GA as determined by another clinical technique. In some embodiments, the GA determined by the process is or is on average within or within about 1% of the GA as determined by another clinical technique.

C. Prenatal Care and Prenatal Clinical Treatments

Also provided herein in some embodiments are methods of screening a pregnant subject, wherein the pregnant subject is selected as a candidate for or eligible for a prenatal care or prenatal clinical treatment based on the results of classifying or determining the GA of the pregnancy of a subject, such as in accord with any of the above provided methods. In some embodiments, the methods of screening a pregnant subject include carrying out any of the provided methods to classify or determine the GA of the pregnancy. Exemplary methods for classifying the GA of the pregnancy are described in Section III-A. Exemplary methods for determining the GA of the pregnancy are described in Section III-B. Information about the GA of the pregnancy can inform or facilitate decisions about prenatal care or prenatal clinical treatments for the subject, such as by excluding subjects who would not be a suitable candidate or eligible for a particular care by virtue of the GA of the pregnancy. It is within the level of skill of a medical professional to determine particular predetermined GA cutpoints that would be useful to assess if a subject is eligible or not eligible for particular prenatal care, prenatal clinical treatments, or assessments. Exemplary methods and GA cutpoints are provided.

Also provided herein in some embodiments are methods of selecting a prenatal care or prenatal clinical treatment for a pregnant subject based on the results of classifying or determining the GA of the pregnancy of a subject, such as in accord with any of the above provided methods. In some embodiments, the methods of selecting a prenatal care or prenatal clinical treatment include carrying out any of the provided methods to classify or determine the GA of the pregnancy. Exemplary methods for classifying the GA of the pregnancy are described in Section III-A. Exemplary methods for determining the GA of the pregnancy are described in Section III-B. In some embodiments, the accurate determination of the GA of a pregnant subject allows for the selection of prenatal care or prenatal clinical treatments suitable for the pregnant subject at that GA. Exemplary prenatal care or prenatal clinical treatments include nutritional interventions, maternal and/or fetal assessment, preventive measures, and interventions for common physiological symptoms.

In some embodiments, the provided methods can be carried out as part of a point-of-care test. In some embodiments, the provided methods can be carried out by a skilled technician in a laboratory as part of a laboratory test. In some embodiments, the provided methods can be carried out without an ultrasound or without other intrusive or expensive clinical assessment.

In some embodiments, the provided methods are used to guide decisions related to prenatal care (e.g., prenatal care activities) or prenatal clinical treatments to be performed during various GA time windows throughout pregnancy, for instance during one or more prenatal visits to a medical provider. Prenatal care activities or prenatal clinical treatments and their associated GA time windows are known in the art (see, e.g., Fescina et al., Montevideo: CLAP/WP; 2009 (CLAP/WR. Scientific Publication; 1562.2); and “WHO recommendations on antenatal care for a positive pregnancy experience”, World Health Organization; both of which are incorporated by reference herein in their entirety). In some embodiments, the GA time window is any time before a GA of or of about 20 weeks, and the activities include pregnancy testing, amenorrhea calculation, perinatal clinical record and risk assessment, exhaustive clinical examination, body weight assessment, height assessment, risky lifestyle investigation, rubeolla susceptibility detection, anti-tetanic vaccine administration, oral examination, breast examination, gynecological examination, PAP, colposcopy, blood group and RH factor assessment, toxoplasmosis detection, HIV detection, hemoglobin assays, iron and folic acid supplementation, syphilis detection, Chagas' Disease detection, malaria detection, urine culture, diabetes detection, education for childbirth and breastfeeding, and any combination of any of the foregoing. In some embodiments, the GA time window is any time between a GA of or of about 22 weeks and a GA of or of about 24 weeks, and the activities include amenorrhea calculation, perinatal clinical record and risk assessment, body weight assessment, risky lifestyle investigation, anti-tetanic vaccine administration, iron and folic acid supplementation, education for childbirth and breastfeeding, blood pressure assessment, fetal growth assessment, fetal livelihood diagnosis, and any combination of any of the foregoing. In some embodiments, the GA time window is any time between a GA of or of about 27 weeks and a GA of or of about 29 weeks, and the activities include amenorrhea calculation, perinatal clinical record and risk assessment, body weight assessment, anti-tetanic vaccine administration, toxoplasmosis detection, hemoglobin assays, iron and folic acid supplementation, urine culture, education for childbirth and breastfeeding, blood pressure assessment, fetal growth assessment, fetal livelihood diagnosis, and any combination of any of the foregoing. In some embodiments, the GA time window is any time between a GA of or of about 33 weeks and a GA of or of about 35 weeks, and the activities include amenorrhea calculation, perinatal clinical record and risk assessment, body weight assessment, risky lifestyle investigation, hemoglobin assays, iron and folic acid supplementation, syphilis detection, diabetes detection, B. Streptococcus infection detection, education for childbirth and breastfeeding, blood pressure assessment, fetal growth assessment, fetal livelihood diagnosis, and any combination of any of the foregoing. In some embodiments, the GA time window is any time between a GA of or of about 38 weeks and a GA of or of about 40 weeks, and the activities include amenorrhea calculation, perinatal clinical record and risk assessment, body weight assessment, iron and folic acid supplementation, education for childbirth and breastfeeding, blood pressure assessment, fetal growth assessment, fetal livelihood diagnosis, pelvic capacity assessment, contraceptive counseling and provision, and any combination of any of the foregoing.

In some embodiments, if the GA of the pregnancy is classified as below a predetermined cutpoint GA, the subject is selected as eligible for certain clinical or prenatal care decision. In other embodiments, if the GA of the pregnancy is classified as above the predetermined cutpoint GA, the subject is excluded from or ineligible for certain prenatal care activities or prenatal clinical treatments and/or requires further tests or assessment prior to certain prenatal care actions or prenatal clinical treatments.

In some embodiments, if the GA of the pregnancy is classified as below the predetermined GA cutpoint, the subject is eligible for or selected as eligible for a particular prenatal care or prenatal clinical treatment. Thus, also provided are methods for screening a subject for a prenatal care or clinical treatment or assessment involving classifying the GA of the pregnancy of the subject according to any of the provided methods, and if the GA is classified as less than the predetermined GA cutpoint, the subject is selected as eligible for a particular prenatal care or prenatal clinical treatment.

In some embodiments, the GA cutpoint is dependent on the particular prenatal care, prenatal clinical treatment, or other medically related decision. In some embodiments, the GA cutpoint is a timepoint before which the pregnant subject is eligible for a prenatal care or prenatal clinical treatment. In some embodiments, the GA cutpoint is a timepoint after which the pregnant subject is eligible for a prenatal care or prenatal clinical treatment. In some embodiments, the GA cutpoint is a timepoint before which a prenatal care or prenatal clinical treatment is appropriate for administration. In some embodiments, the GA cutpoint is a timepoint after which a prenatal care or prenatal clinical treatment is appropriate for administration.

In some embodiments, the provided methods are used to guide decisions related to screening based on a GA cutpoint at the end of embryogenesis, i.e. a GA cutpoint at or about 10 weeks or 70 days. In particular aspects, information about whether a pregnancy is at the end of embryogenesis is relevant for a variety of clinical or prenatal care decisions. In some embodiments, the provided methods are useful for assessing eligibility for early aspiration or outpatient medical abortion. In some embodiments, the provided methods are used to assess eligibility for medical abortions. In some embodiments, the medical abortion is early aspiration. In some embodiments, the medical abortion is outpatient medical abortion. In particular embodiments, a GA cutpoint of or of about 9 weeks (63 days), 10 weeks (70 days), or 11 weeks (77 days) is used to screen subjects for eligibility or ineligibility for a medical abortion. In some embodiments, a GA cutpoint of or of about 9 weeks (63 days) is used to screen subjects for eligibility or ineligibility for a medical abortion. In some embodiments, a GA cutpoint of or of about 10 weeks (70 days) is used to screen subjects for eligibility or ineligibility for a medical abortion. In some embodiments, a GA cutpoint of or of about 11 weeks (77 days) is used to screen subjects for eligibility or ineligibility for a medical abortion.

In some embodiments, the provided methods are used to screen subjects that may be susceptible to or at risk of embryotoxicity. In particular embodiments, embryotoxicity is of primary concern within the first 8-10 weeks of gestation. In some embodiments, the provided methods identify pregnancies that have progressed beyond that point in order to clear a female subject from being able to engage in certain activities which may not be desired at earlier weeks of gestation, such as due to risk from potentially dangerous exposures. In such embodiments, a GA cutpoint of or of about 8 weeks, 9 weeks, 10 weeks, or any value between any of the foregoing would be useful to distinguish earlier and later pregnancies based on embryotoxicity risk.

In some embodiments, the provided methods are used to screen subjects that may be eligible or ineligible for certain diagnostic or clinical tests or procedures. For example, early in pregnancy, such as prior to the end of embryogenesis, it may not be desirable to carry out certain tests or procedures. In some embodiments, the test or procedure relates to a fetal or maternal screening. In some embodiments, the test or procedure is amniocentesis. In some embodiments, the test or procedure is a glucose tolerance test. In some embodiments, the test or procedure is or includes an ultrasound.

In particular embodiments, the provided methods are used to screen subjects that may be eligible or ineligible for a test for chromosomal abnormalities, in some embodiments, the provided methods are carried out to determine if a woman is suitable for amniocentesis, which, in some cases, is offered to pregnant subjects between 15 and 18 weeks of gestation age. In some embodiments, the provided methods are used to assess suitability for certain prenatal screening tests, including prenatal tests screening for a chromosomal abnormality chromosomal, such as trisomy-13, trisomy-18, Turner syndrome, Klinefelter syndrome, or trisomy 21(Down syndrome). In some embodiments, a GA cutpoint of or of about 10 weeks or later, such as at or about 15 weeks (105 days), at or about 16 weeks, at or about 17 weeks, or at or about 18 weeks, or any value between any of the foregoing is used to screen if the subject is eligible for tests to assess chromosomal abnormalities or that involve amniocentesis. In some embodiments, a GA cutpoint of or of about 104 days or of or of about 105 days or later is used to screen if the subject is eligible for tests to assess chromosomal abnormalities or that involve amniocentesis.

In particular embodiments, the provided methods are used to screen subjects that many be eligible or ineligible for a glucose tolerance test, which is a test that is normally not carried out until 26-28 weeks of gestation. In such embodiments, a GA cutpoint of or of about 20 weeks (140 days) or later, such as at or about 26 weeks, at or about 27 weeks, at or about 28 weeks, or any value between any of the foregoing is used to screen if the subject is eligible for a glucose tolerance test.

### IV. Devices, Kits, Articles of Manufacture, And Systems

Provided herein in some embodiments are devices for carrying out any of the provided methods. In some embodiments, the device is suitable for performing any of the methods described herein of detecting PAPP-A in a biological sample from a pregnant subject. In some embodiments, the device is further suitable for measuring the amount or concentration of PAPP-A in the biological sample. In some embodiments, the device is further suitable for classifying or determining GA in accordance with any of the methods provided herein.

In some embodiments, the device includes an antibody or antibody fragment for detecting or capturing PAPP-A. In some embodiments, the device is used in conjunction with an antibody or antibody fragment for detecting or capturing PAPP-A. Exemplary antibodies or antibody fragments include any as described in Section II. In some embodiments, the antibody is a capture antibody. In some embodiments, the antibody, e.g., the capture antibody, is capable of specifically binding to homodimeric PAPP-A and the heterotetrameric PAPP-A/proMBP complex, for instance equimolarly or about equimolarly. In some embodiments, the device further includes a detection antibody capable of specifically binding to homodimeric PAPP-A and the heterotetrameric PAPP-A/proMBP complex, for instance equimolarly or about equimolarly. Exemplary capture and detection antibodies are described in Section II.

In some embodiments, the biological sample is a whole blood sample. In some embodiments, the biological sample is a serum sample. In some embodiments, the biological sample is any as described in Section II-A.

In some embodiments, the device is a diagnostic device. In some embodiments, the device is a point-of-care device or is suitable for use as a point-of-care device. In other embodiments, the device is for use in a medical or hospital laboratory.

In some embodiments, the device is a stationary device. In other embodiments, the device is a portable device. In some embodiments, the device is the size of a desktop printer or smaller. In some embodiments, the device is a handheld device. In some embodiments, the device is manually operated. In other embodiments, the device is automatically operated. In some embodiments, the device is an electronic device.

In some embodiments, the device is suitable for performing a sandwich lateral flow assay. In some embodiments, the device is a lateral flow device. In some embodiments, the device is a lateral flow stick. Such devices are known and are described in, for example, U.S. Pat. No. 10,203,340; EP 0291194; WO 1995/013542; U.S. Pat. Nos. 5,504,013; and 6,156,271, the contents of each of which are incorporated by reference in their entirety. For instance, in some embodiments the device is a lateral flow strip that includes a sampling portion and a test zone in fluid communication with the sampling portion. In some embodiments, the lateral flow strip is a nitrocellulose lateral flow strip. In some embodiments, the test sample is applied to the sampling portion, from which the fluid can flow to the test zone. In some embodiments, the test zone includes the detection antibody, and the capture antibody is immobilized in the test zone. In some embodiments, the test zone is the zone in which the complex that includes the capture antibody, the detection antibody, and PAPP-A will form and accumulate, and from which the detectable signal produced by the detection antibody will be assessed. In some embodiments, the degree of the detectable signal is assessed visually. In some embodiments, the lateral flow strip includes means for detecting, measuring, or quantitating the degree of the detectable signal. In some embodiments, the lateral flow strip is inserted into a separate device capable of detecting, measuring, or quantitating the degree of the detectable signal.

Any device suitable for use with the solid supports described herein or for use in the methods provided herein can be used. Non-limiting examples of suitable devices are described below and include commercially available devices such as i-STAT® handheld (Abbott), Minicare 1-20 (Phillips), and similar handheld devices.

In some embodiments, the device includes a solid support to which the capture antibody or antibody fragment for capturing PAPP-A can be attached or immobilized. In some embodiments, the device is used in conjunction with a solid support to which the capture antibody or antibody fragment for capturing PAPP-A can be attached or immobilized. In some embodiments, the solid support is a bead, column, array, assay plate, microwell, cartridge, stick, filter, or strip. In some embodiments, the solid support is formed of glass (e.g., controlled pore glass), polysaccharides (e.g., agarose), polyacrylamides, polystyrene, polyvinyl alcohol, nitrocellulose, cellulose, nylon, silicones, or other materials well known in the art to which antibodies or antibody fragments can be directly or indirectly attached or immobilized. In some embodiments, the capture antibody or antibody fragment is attached or immobilized to the solid support.

In some embodiments, the device further includes the detection antibody. In some embodiments, the detection antibody is not immobilized to the solid support.

In some embodiments, the solid support is the device. In some embodiments, the solid support is inserted into the device, attached to the device, and/or held by the device in order for the device to be operable.

In some embodiments, the solid support is inserted into the device, attached to the device, and/or held by the device when the device is operating for measuring PAPP-A in the biological sample. In some embodiments, the solid support is configured in the device to receive a sample loaded into the device. In some embodiments, the sample is added to the solid support prior to its insertion or prior to attachment of the solid support with or into the device.

In some embodiments, the device is further configured to add one or more solutions from one or more dispensers into the solid support. In some embodiments, the device is further configured to remove solution from the solid support. In some embodiments, the device is configured to hold the one or more solutions and to individually dispense each solution at an appropriate time into the solid support held or inserted in the device.

In some embodiments, the one or more solutions includes a solution containing one or more detection reagents. In some embodiments, the one or more detection reagents include an antibody or antibody fragment for detecting PAPP-A. Exemplary antibodies or antibody fragments include any as described in Section II. In some embodiments, the antibody or antibody fragment used as a detection reagent binds to a different epitope than does the antibody or antibody fragment used to capture PAPP-A, e.g., the antibody or antibody fragment attached or immobilized on the solid support.

In some embodiments, the one or more solutions includes a wash solution. In some embodiments, the one or more solutions includes a substrate or stop solution.

In some embodiments, the device is used to assess PAPP-A using one or more techniques well known in the art, such as spectrophotometry, high performance liquid chromatography (HPLC), immunoassays such as enzyme-linked immunosorbent assay (ELISA), Western blot, automated imaging, immunohistochemistry, flow cytometry, high-throughput screening of an array such as a microarray or nanoarray, and surface plasmon resonance. In some embodiments, the device is used to assess PAPP-A using an immunoassay. In some embodiments, the device is used to assess PAPP-A using an ELISA. In some embodiments, the device is used to measure PAPP-A using a sandwich ELISA. In some embodiments, the device includes a system for reading an assay output, such as a fluorometer, luminometer, or spectrophotometer for assay detection.

In some embodiments, the device automates or partially automates an assay method that detects a particular biomarker or biomarkers, such as any of the methods provided herein that detects PAPP-A as described. In some embodiments, actions that are automated by the instrument include mixing or agitation of a sample during an incubation phase, dispensing or adding one or more solutions, washing of a sample, controlling incubation times, optical illumination and/or reading of an assay, and calculation of a biomarker amount or concentration in the sample. In some embodiments, the timing of any of the above automated steps is preset or predetermined, such as to assay specific guidelines.

In some embodiments, the device includes a computing system or processor. In some embodiments, the computing system includes one or more computer executable logics (e.g., one or more computer programs) that are recorded on a computer readable medium. In some embodiments, the computing system is configured to execute any function used as part of any of the methods provided herein. In some embodiments, the computing system or processor is configured to execute some or all of the following functions: (i) processing a signal representative of the detected PAPP-A; (ii) comparing data as detected from the sample with a reference standard; (iii) calculating an amount or concentration of PAPP-A in the biological sample; and/or (iv) displaying or outputting a value representative of the calculated amount or concentration of PAPP-A. In some embodiments, the computing system or processor is further configured to (i) compare the calculated amount or concentration of PAPP-A to that in a second sample, e.g., a reference PAPP-A sample, and/or (ii) display or output a message based on the comparison. In some embodiments, the computing system or processor is further configured to (i) provide the calculated amount or concentration of PAPP-A as input to a process that uses PAPP-A amount or concentration as a continuous predictor of GA, e.g., any of the processes described in Section III-B, and/or (ii) display the GA predicted by the process based on the calculated amount or concentration of PAPP-A. In some embodiments, the computing system is configured to perform any one of the methods described herein.

In some embodiments, the computer executable logic works in any computer that can be any of a variety of types of general-purpose computers, such as a personal computer, network server, workstation, handheld device, or other computer platform now or later developed. In some embodiments, a computing system includes a computer usable medium having the computer executable logic (computer software program, including program code) stored therein. In some embodiments, the computer executable logic is executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts.

Also provided herein in some embodiments are articles of manufacture or kits for carrying out any of the provided methods. In some embodiments, the article of manufacture or kit is compatible for operation in connection with a system or device for detecting PAPP-A in a biological sample. In some embodiments, the device is any as described herein. In some embodiments, the article of manufacture or kit further includes any of the devices provided herein.

In some embodiments, the article of manufacture or kit includes a solid support to which an antibody or antibody fragment for capturing PAPP-A can be attached or immobilized. Exemplary antibodies or antibody fragments include any as described in Section II. In some embodiments, the solid support is any as described herein. In some embodiments, the article of manufacture or kit includes instructions for attaching the antibody or antibody fragment to the solid support. In some embodiments, the article of manufacture or kit includes the antibody or antibody fragment attached directly or indirectly to the solid support.

In some embodiments, the article of manufacture or kit contains reagents for detecting or capturing PAPP-A in the sample. In some embodiments, the reagents include the antibody or antibody fragment for capturing PAPP-A, e.g., the antibody or antibody fragment to be attached or immobilized on the solid support. In some embodiments, the article of manufacture or kit includes a composition containing the antibody or antibody fragment for capturing PAPP-A. In some embodiments, the article of manufacture or kit includes a composition containing detection reagents, such as antibodies, buffers, substrates for enzymatic staining, and/or chromagens. In some embodiments, the detection reagents include an antibody or antibody fragment for detecting PAPP-A, e.g., any as described in Section II. In some embodiments, the antibody or antibody fragment used as a detection reagent binds to a different epitope than does the antibody or antibody fragment used to capture PAPP-A.

In some embodiments, the article of manufacture or kit contains reagents for dilution of the biological sample in accord with the methods provided herein. In some embodiments, the article of manufacture or kit includes a suitable diluent. In some embodiments, the article of manufacture or kit contains sterilized water or saline solutions for dilution of biological samples.

In some embodiments, the article of manufacture or kit contains a reference PAPP-A sample for use in accord with any of the provided methods. In some embodiments, the reference PAPP-A sample is any as described in Section III-A.

In some embodiments, the article of manufacture or kit includes one or more additional reagents or components desirable from a commercial, therapeutic, and/or user standpoint for performing the provided methods. Exemplary additional reagents or components include secondary antibodies, affinity labels, capture reagents, buffers, diluents, signal detection agents, filters, needles, syringes, capillary tubes, and package inserts with instructions for use. In some embodiments, the article of manufacture or kit further contains reagents for collection of biological samples. In some embodiments, the article of manufacture or kit further contains reagents for preparation and processing of biological samples. In some embodiments, the article of manufacture or kit further contains additional devices for practice of the provided methods. In some embodiments, the article of manufacture or kit further contains additional components, such as tubes, containers, syringes, slides, containers, and microtiter plates, for practice of the provided methods. Those of skill in the art will recognize many other possible containers, plates, and reagents that can be used in accord with the provided methods.

In some embodiments, the kits are provided as articles of manufacture that include packing materials for the packaging of the antibodies or compositions thereof and/or the one or more additional reagents or components. For example, the kits can contain containers, bottles, tubes, vials, and any packaging material suitable for separating or organizing the components of the kit.

In some embodiments, the kit includes one or more containers. Suitable containers include, for example, bottles, vials (e.g., dual chamber vials), syringes (such as single or dual chamber syringes), and test tubes. The one or more containers can be formed from a variety of materials, such as glass or plastic. In some embodiments, the one or more containers include a single-use vial or a multi-use vial.

In some embodiments, the one or more containers hold a composition that includes antibodies or other reagents for use in the provided methods. In some embodiments, the one or more containers hold a composition that includes the antibody or antibody fragment for capturing PAPP-A. In some embodiments, the one or more containers hold a composition that includes the antibody or antibody fragment for detecting PAPP-A. In some embodiments, the one or more containers hold a composition that includes other reagents for detecting PAPP-A, for instance other antibodies, buffers, substrates for enzymatic staining, and/or chromagens. The article of manufacture or kit provided herein can include the antibodies or reagents in separate containers or in the same container.

In some embodiments, the article of manufacture or kit further includes a container holding a diluent suitable for use in the provided methods.

In some embodiments, the article of manufacture or kit further includes a container holding a reference PAPP-A sample for use in the provided methods. In some embodiments, the reference PAPP-A sample is any as described in Section III-A. In some embodiments, the article of manufacture or kit includes two or more such reference PAPP-A samples.

In some embodiments, the kit includes instructions. Instructions typically include a tangible expression describing the antibodies, other components included in the kit, and/or methods for using the antibodies to detect or measure PAPP-A in a biological sample, such as in accord with any of the provided methods. In some embodiments, the instructions are provided as a label or a package insert, which is on or associated with the container. In some embodiments, the instructions indicate directions for reconstitution and/or use of compositions containing antibodies or detection reagents.

### V. Exemplary Embodiments

Among the provided embodiments are:
1. A method of detecting PAPP-A in a biological sample from a pregnant subject, comprising:
(a) preparing a test sample from a pregnant female subject, the preparing comprising:
   (i) obtaining a biological sample from the pregnant female subject, wherein the obtained biological sample is a whole blood, serum, or plasma sample and wherein the obtained biological sample has a volume between or between about 0.5 μL and 10 μL, inclusive; and
   (ii) diluting the volume of the obtained biological sample in a sample diluent between or between about 2-fold and 100-fold, inclusive, thereby preparing the test sample; and
(b) detecting in the test sample one or more PAPP-A proteoforms using an immunoassay, said one or more PAPP-A proteoforms comprising homodimeric PAPP-A and/or a heterotetrameric PAPP-A/proMBP complex, wherein the immunoassay comprises:
   (i) contacting the test sample with an antibody capable of specifically binding to (1) homodimeric PAPP-A and (2) the heterotetrameric PAPP-A/proMBP complex under conditions to form a complex comprising the antibody and the one or more PAPP-A proteoforms; and
   (ii) detecting the complex comprising the antibody and the one or more PAPP-A proteoforms.
2. The method of embodiment 1, wherein when the biological sample is obtained, the gestational age (GA) of the pregnant female subject is suspected to be between or between about 5 weeks and 40 weeks, between or between about 5 weeks and 30 weeks, or between or between about 5 weeks and 20 weeks, each inclusive.
3. The method of embodiment 1, wherein when the biological sample is obtained, the GA of the pregnant female subject is suspected to be between or between about 5 weeks and 15 weeks, inclusive.
4. The method of any of embodiments 1-3, wherein the immunoassay is a colorimetric assay.
5. The method of any of embodiments 1-4, wherein the immunoassay is a solid-phase immunoassay, optionally wherein the antibody is immobilized on a solid support.
6. The method of any of embodiments 1-5, wherein the immunoassay is an Enzyme linked immunosorbent assay (ELISA), optionally a sandwich ELISA
7. The method of any of embodiments 1-5, wherein the immunoassay is a lateral flow assay.
8. The method of any of embodiments 1-7, wherein the antibody is capable of specifically binding to (1) homodimeric PAPP-A and (2) the heterotetrameric PAPP-A/proMBP complex equimolarly or about equimolarly.
9. The method of any of embodiments 1-8, wherein the antibody is a capture antibody and the complex is a first complex comprising the capture antibody and the one or more PAPP-A proteoforms, and wherein:
prior to the detecting, the immunoassay further comprises contacting the first complex comprising the capture antibody and the one or more PAPP-A proteoforms with a detection antibody capable of specifically binding to (1) homodimeric PAPP-A and (2) the heterotetrameric PAPP-A/proMBP complex under conditions to form a second complex comprising the capture antibody, the one or more PAPP-A proteoforms, and the detection antibody; and
the detecting comprises detecting the second complex.
10. The method of any of embodiments 1-8, wherein the antibody is a detection antibody and the complex is a first complex comprising the detection antibody and the one or more PAPP-A proteoforms, and wherein:
prior to the detecting, the immunoassay further comprises contacting the first complex comprising the detection antibody and the one or more PAPP-A proteoforms with a capture antibody capable of specifically binding to (1) homodimeric PAPP-A and (2) the heterotetrameric PAPP-A/proMBP complex under conditions to form a second complex comprising the capture antibody, the one or more PAPP-A proteoforms, and the detection antibody; and
the detecting comprises detecting the second complex.
11. The method of embodiment 9 or embodiment 10, wherein the detection antibody is conjugated to a detectable label capable of producing a detectable signal, and the detecting the second complex is by assessing the degree of the detectable signal produced by the detectable label.
12. The method of any of embodiments 1-11, further comprising determining the concentration of PAPP-A in the obtained biological sample by comparison of the degree of the detectable signal assessed from the test sample to a standard curve.
13. A method of detecting PAPP-A in a biological sample from a pregnant subject, comprising:
(a) preparing a test sample from a pregnant female subject, the preparing comprising:
   (i) obtaining a biological sample from the pregnant female subject, wherein the obtained biological sample is a whole blood, serum, or plasma sample and wherein the obtained biological sample has a volume between or between about 0.5 μL and 10 μL, inclusive; and
   (ii) diluting the volume of the obtained biological sample in a sample diluent between or between about 2-fold and 100-fold, inclusive, thereby preparing the test sample; and
(b) detecting in the test sample one or more PAPP-A proteoforms using an immunoassay, said one or more PAPP-A proteoforms comprising homodimeric PAPP-A and/or a heterotetrameric PAPP-A/proMBP complex, wherein the immunoassay comprises:
   (i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and
   (ii) assessing the degree of the detectable signal produced by the detectable label.
14. The method of embodiment 13, wherein the contacting of the test sample with the capture antibody and the detection antibody is carried out simultaneously.
15. The method of embodiment 13, wherein the contacting of the test sample with the capture antibody and the detection antibody is carried out sequentially, in either order.
16. The method of embodiment 13 or embodiment 15, wherein the test sample is contacted with the capture antibody prior to being contacted with the detection antibody, wherein:
the test sample is contacted with the capture antibody under conditions to form a first complex comprising the capture antibody and the one or more PAPP-A proteoforms; and
the first complex is contacted with the detection antibody under conditions to form a second complex comprising the capture antibody, the one or more PAPP-A proteoforms, and the detection antibody.
17. The method of embodiment 13 or embodiment 15, wherein the test sample is contacted with the detection antibody prior to being contacted with the capture antibody, wherein:
the test sample is contacted with the detection antibody under conditions to form a first complex comprising the detection antibody and the one or more PAPP-A proteoforms; and
the first complex is contacted with the capture antibody under conditions to form a second complex comprising the capture antibody, the one or more PAPP-A proteoforms, and the detection antibody.
18. The method of embodiment any of embodiments 13-17, wherein when the biological sample is obtained, the gestational age (GA) of the pregnant female subject is suspected to be between or between about 5 weeks and 40 weeks, between or between about 5 weeks and 30 weeks, or between or between about 5 weeks and 20 weeks, or between or between about 5 weeks and 15 weeks, each inclusive.
19. The method of any of embodiments 13-18, wherein the immunoassay is a colorimetric assay.
20. The method of any of embodiments 13-19, wherein the immunoassay is a solid-phase immunoassay.
21. The method of any of embodiments 13-20, wherein the immunoassay is an Enzyme linked immunosorbent assay (ELISA), optionally a sandwich ELISA.
22. The method of any of embodiments 13-20, wherein the immunoassay is a lateral flow assay.
23. The method of any of embodiments 11-22, the method further comprising classifying the GA of the pregnant female subject based on the degree of the detectable signal assessed from the test sample.
24. The method of any of embodiments 11-22, further comprising determining the concentration of PAPP-A in the obtained biological sample by comparison of the degree of the detectable signal measured from the test sample to a standard curve.
25. The method of embodiment 12 or embodiment 24, the method further comprising classifying the GA of the pregnant female subject based on the concentration of PAPP-A in the obtained biological sample.
26. A method for classifying the gestational age (GA) of a pregnancy, comprising:
(a) detecting PAPP-A in a biological sample obtained from a pregnant female subject according to the method of any of embodiments 11-25;
(b) comparing the degree of the detectable signal assessed from the test sample to the degree of a detectable signal assessed using the immunoassay from a reference PAPP-A sample, wherein the reference PAPP-1 sample comprises (1) homodimeric PAPP-A and/or (2) the heterotetrameric PAPP-A/proMBP complex, and wherein the concentration of the reference PAPP-A sample is a predetermined concentration associated with a predetermined GA cutpoint using the immunoassay; and
(c) classifying:
   the GA of the pregnancy as less than the predetermined GA cutpoint if the degree of the detectable signal assessed from the test sample is lower than the degree of the detectable signal assessed from the reference sample; or
   the GA of the pregnancy as greater than or equal to the predetermined GA cutpoint if the degree of the detectable signal assessed from the test sample is higher than or equal to the degree of the detectable signal assessed from the reference sample.
27. The method of embodiment 26, wherein the reference PAPP-A sample comprises the heterotetrameric PAPP-A/proMBP complex.
28. A method for classifying the gestational age (GA) of a pregnancy, comprising:
(a) detecting PAPP-A in a biological sample obtained from a pregnant female subject using an immunoassay, wherein:
   the biological sample is a whole blood, serum, or plasma sample; and
   the immunoassay comprises detecting one or more PAPP-A proteoforms in a test sample derived from the biological sample, said one or more PAPP-A proteoforms comprising homodimeric PAPP-A or a heterotetrameric PAPP-A/proMBP complex and said detecting comprising:
      (i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and
      (ii) assessing the degree of the detectable signal produced by the detectable label;
(b) comparing the degree of the detectable signal assessed from the test sample to the degree of a detectable signal measured using the immunoassay from a reference PAPP-A sample, wherein the reference PAPP-A sample comprises the heterotetrameric PAPP-A/proMBP complex, and wherein the concentration of the reference PAPP-A sample is a predetermined concentration associated with a predetermined GA cutpoint; and
(c) classifying:
   the GA of the pregnancy as less than the predetermined GA cutpoint if the degree of the detectable signal assessed from the test sample is lower than the degree of the detectable signal assessed from the reference sample; or
   the GA of the pregnancy as greater than or equal to the predetermined GA cutpoint if the degree of the detectable signal assessed from the test sample is higher than or equal to the degree of the detectable signal assessed from the reference sample.
29. The method of embodiment 27 or embodiment 28, wherein the reference PAPP-A sample further comprises homodimeric PAPP-A.
30. The method of any of embodiments 27-29, wherein the reference PAPP-A sample consists essentially of the heterotetrameric PAPP-A/proMBP complex.
31. A method for classifying the gestational age (GA) of a pregnancy, comprising:
(a) determining the concentration of PAPP-A in a biological sample obtained from a pregnant female subject according to the method of embodiment 12 or embodiment 24;
(b) comparing the concentration of PAPP-A in the obtained biological sample to a predetermined concentration of PAPP-A, wherein the predetermined concentration is associated with a predetermined GA cutpoint using the immunoassay; and
(c) classifying:
   the GA of the pregnancy as less than a predetermined GA cutpoint if the concentration of PAPP-A in the obtained biological sample is lower than the predetermined concentration; or
      the GA of the pregnancy as greater than or equal to the predetermined GA cutpoint if the concentration of PAPP-A in the obtained biological sample is higher than or equal to the predetermine concentration.
32. The method of any of embodiments 26-31, wherein the predetermined GA cutpoint is a timepoint between or between about 5 weeks and 40 weeks, between or between about 5 weeks and 30 weeks, or between or between about 5 weeks and 20 weeks, each inclusive.
33. The method of any of embodiments 26-31, wherein the predetermined GA cutpoint is a timepoint between or between about 5 weeks and 15 weeks, inclusive.
34. The method of any of embodiments 26-31, wherein the predetermined GA cutpoint is a timepoint between or between about 60 days and 140 days, inclusive.
35. The method of any of embodiments 26-31, wherein the predetermined GA cutpoint is a timepoint between or between about 56 days and 84 days, inclusive.
36. The method of any of embodiments 26-31, wherein the predetermined GA cutpoint is or is about 56 days, 63 days, 70 days, 77 days, or 84 days.
37. The method of any of embodiments 26-31, wherein the predetermined GA cutpoint is a timepoint between or between about 63 days and 77 days, inclusive.
38. The method of any of embodiments 26-31, wherein the predetermined GA cutpoint is or is about 63 days, 70 days, or 77 days.
39. The method of any of embodiments 26-31, wherein the predetermined GA cutpoint is or is about 56 days.
40. The method of any of embodiments 26-33, 35, 36, and 39, wherein the predetermined concentration is between or between about 1 ng/mL and 70 ng/mL, inclusive.
41. The method of any of embodiments 26-33, 35, 36, and 39, wherein the predetermined concentration is between or between 5 ng/mL and 55 ng/mL, inclusive.
42. The method of any of embodiments 26-31, wherein the predetermined GA cutpoint is or is about 63 days.
43. The method of any of embodiments 26-38 and 42, wherein the predetermined concentration is between or between about 25 ng/mL and 150 ng/mL, inclusive.
44. The method of any of embodiments 26-38 and 42, wherein the predetermined concentration is between or between 40 ng/mL and 130 ng/mL, inclusive.
45. The method of any of embodiments 26-31, wherein the predetermined GA cutpoint is or is about 70 days.
46. The method of any of embodiments 26-38 and 45, wherein the predetermined concentration is between or between about 60 ng/mL and 200 ng/mL, inclusive.
47. The method of any of embodiments 26-38 and 45, wherein the predetermined concentration is between or between 70 ng/mL and 185 ng/mL, inclusive.
48. The method of any of embodiments 26-31, wherein the predetermined GA cutpoint is or is about 77 days.
49. The method of any of embodiments 26-38 and 48, wherein the predetermined concentration is between or between about 80 ng/mL and 220 ng/mL, inclusive.
50. The method of any of embodiments 26-38 and 48, wherein the predetermined concentration is between or between 90 ng/mL and 215 ng/mL, inclusive.
51. The method of any of embodiments 26-31, wherein the predetermined GA cutpoint is or is about 84 days.
52. The method of any of embodiments 26-36 and 51, wherein the predetermined concentration is between or between about 160 ng/mL and 480 ng/mL, inclusive.
53. The method of any of embodiments 26-36 and 51, wherein the predetermined concentration is between or between 170 ng/mL and 470 ng/mL, inclusive.
54. The method of any of embodiments 26-31, wherein the predetermined GA cutpoint is a timepoint between or between about 98 days and 112 days, inclusive.
55. The method of any of embodiments 26-31, wherein the predetermined GA cutpoint is or is about 98 days, 105 days, or 112 days.
56. The method of any of embodiments 26-31, wherein the predetermined GA cutpoint is or is about 98 days.
57. The method of any of embodiments 26-34 and 56, wherein the predetermined concentration is between or between about 550 ng/mL and 1240 ng/mL, inclusive.
58. The method of any of embodiments 26-34 and 56, wherein the predetermined concentration is between or between 565 ng/mL and 1230 ng/mL, inclusive.
59. The method of any of embodiments 26-31, wherein the predetermined GA cutpoint is or is about 105 days.
60. The method of any of embodiments 26-34 and 59, wherein the predetermined concentration is between or between about 1440 ng/mL and 1490 ng/mL, inclusive.
61. The method of any of embodiments 26-34 and 59, wherein the predetermined concentration is between or between 1450 ng/mL and 1475 ng/mL, inclusive.
62. The method of any of embodiments 26-31, wherein the predetermined GA cutpoint is or is about 112 days.
63. The method of any of embodiments 26-32 and 62, wherein the predetermined concentration is between or between about 1440 ng/mL and 1490 ng/mL, inclusive.
64. The method of any of embodiments 26-32 and 62, wherein the predetermined concentration is between or between 1450 ng/mL and 1475 ng/mL, inclusive.
65. The method of any of embodiments 26-31, wherein the predetermined GA cutpoint is or is about 168 days.
66. The method of any of embodiments 26-32 and 65, wherein the predetermined concentration is between or between about 3500 ng/mL and 4500 ng/mL, inclusive.
67. The method of any of embodiments 26-32 and 65, wherein the predetermined concentration is between or between 4000 ng/mL and 4200 ng/mL, inclusive.
68. The method of any of embodiments 23 and 25-67, wherein the classifying is performed with greater than or greater than about 80% sensitivity and/or greater than or greater than about 80% specificity.
69. The method of any of embodiments 23 and 25-67, wherein the classifying is performed with greater than or greater than about 85% sensitivity and/or greater than or greater than about 85% specificity.
70. The method of any of embodiments 23 and 25-67, wherein the classifying is performed with greater than or greater than about 90% sensitivity and/or greater than or greater than about 90% specificity.
71. A method for screening a pregnant subject for a prenatal care or prenatal clinical treatment, comprising:
(a) classifying the gestational age (GA) of a pregnancy according to the method of any of embodiments 23 and 25-70; and
(b) based on the classifying, (i) selecting the pregnant female subject as eligible for a prenatal care or prenatal clinical treatment if the GA of the pregnancy is classified as less than the predetermined GA cutpoint; or (ii) selecting the pregnant female subject as not eligible for the prenatal care or prenatal clinical treatment or as a candidate for further assessment for the prenatal care or prenatal clinical treatment if the GA of the pregnancy is classified as greater than or equal to the predetermined GA cutpoint.
72. A method for performing a prenatal care or prenatal clinical treatment on a pregnant subject, comprising performing a prenatal care or prenatal clinical treatment on a pregnant female subject selected as eligible for the prenatal care or prenatal clinical treatment according to the method of embodiment 71.
73. A method for determining the gestational age (GA) of a pregnancy, comprising:
(a) detecting PAPP-A in a biological sample obtained from a pregnant female subject according to the method of any of embodiments 11-25; and
(b) determining the GA of the pregnancy based on the degree of the detectable signal assessed from the test sample, wherein the determining comprises providing the degree of the detectable signal assessed from the test sample as input to a process that uses the degree of the detectable signal measured from the test sample to predict GA.
74. A method for determining the gestational age (GA) of a pregnancy, comprising:
(a) detecting PAPP-A in a biological sample obtained from a pregnant female subject using an immunoassay, wherein:
   the biological sample is a whole blood, serum, or plasma sample; and
   the immunoassay comprises detecting one or more PAPP-A proteoforms in a test sample derived from the biological sample, said one or more PAPP-A proteoforms comprising homodimeric PAPP-A or a heterotetrameric PAPP-A/proMBP complex, said detecting comprising:
      (i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and
      (ii) measuring the degree of the detectable signal produced by the detectable label; and
(b) determining the GA of the pregnancy based on the degree of the detectable signal assessed from the test sample, wherein the determining comprises providing the degree of the detectable signal assessed from the test sample as input to a process that uses the degree of the detectable signal assessed from the test sample to predict GA.
75. A method for determining the gestational age (GA) of a pregnancy, comprising:
(a) measuring the concentration of PAPP-A in a biological sample obtained from a pregnant female subject according to the method of embodiment 12 or embodiment 24; and
(b) determining the GA of the pregnancy based on the concentration of PAPP-A in the obtained biological sample, wherein the determining comprises providing the concentration of PAPP-A in the obtained biological sample as input to a process that uses PAPP-A concentration as a continuous predictor of GA.
76. A method for determining the gestational age (GA) of a pregnancy, comprising:
(a) determining the concentration of PAPP-A in a biological sample obtained from a pregnant female subject using an immunoassay, wherein:
   the biological sample is a whole blood, serum, or plasma sample; and
   the immunoassay comprises detecting one or more PAPP-A proteoforms in a test sample derived from the biological sample, said one or more PAPP-A proteoforms comprising homodimeric PAPP-A or a heterotetrameric PAPP-A/proMBP complex, said detecting comprising:
      (i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and
      (ii) assessing the degree of the detectable signal produced by the detectable label; and
      (iii) determining the concentration of PAPP-A in the obtained biological sample by comparison of the degree of the detectable signal to a standard curve; and
(b) determining the GA of the pregnancy based on the concentration of PAPP-A in the obtained biological sample, wherein the determining comprises providing the concentration of PAPP-A in the obtained biological sample as input to a process that uses PAPP-A concentration as a continuous predictor of GAs.
77. The method of any of embodiments 73-76, wherein the contacting of the test sample with the capture antibody and the detection antibody is carried out simultaneously.
78. The method of any of embodiments 73-76, wherein the contacting of the test sample with the capture antibody and the detection antibody is carried out sequentially in either order.
79. The method of any of embodiments 73-76 and 78, wherein the test sample is contacted with the capture antibody prior to being contacted with the detection antibody, wherein:
the test sample is contacted with the capture antibody under conditions to form a first complex comprising the capture antibody and the one or more PAPP-A proteoforms; and
the first complex is contacted with the detection antibody under conditions to form a second complex comprising the capture antibody, the one or more PAPP-A proteoforms, and the detection antibody.
80. The method of any of embodiments 73-76 and 78, wherein the test sample is contacted with the detection antibody prior to being contacted with the capture antibody, wherein:
the test sample is contacted with the detection antibody under conditions to form a first complex comprising the detection antibody and the one or more PAPP-A proteoforms; and
the first complex is contacted with the capture antibody under conditions to form a second complex comprising the capture antibody, the one or more PAPP-A proteoforms, and the detection antibody.
81. The method of any of embodiments 73-80, wherein when the biological sample is obtained, the gestational age (GA) of the pregnant female subject is suspected to be between or between about 5 weeks and 40 weeks, between or between about 5 weeks and 30 weeks, or between or between about 5 weeks and 20 weeks, each inclusive.
82. The method of any of embodiments 73-80, wherein when the biological sample is obtained, the GA of the pregnant female subject is suspected to be between or between about 5 weeks and 15 weeks, inclusive.
83. The method of any of embodiments 73-82, wherein the immunoassay is a colorimetric assay.
84. The method of any of embodiments 73-83, wherein the immunoassay is a solid-phase immunoassay.
85. The method of any of embodiments 73-84, wherein the immunoassay is an Enzyme linked immunosorbent assay (ELISA), optionally a sandwich ELISA.
86. The method of any of embodiments 73-84, wherein the immunoassay is a lateral flow assay.
87. The method of any of embodiments 73-86, wherein the process comprises a regression model trained using GAs and PAPP-A concentrations from a plurality of pregnant female subjects.
88. The method of embodiment 87, wherein the regression model is a linear regression model, a piecewise linear model, a polynomial regression model, or a Bayesian model.
89. The method of any of embodiments 73-88, wherein the process predicts GAs between or between about 5 weeks and 40 weeks, between or between about 5 weeks and 30 weeks, or between or between about 5 weeks and 20 weeks, each inclusive.
90. The method of any of embodiments 73-88, wherein the process predicts GAs between or between about 5 weeks and 15 weeks, inclusive.
91. The method of any of embodiments 73-88, wherein the process predicts GAs between or between about 10 weeks and 15 weeks, inclusive.
92. A method of selecting a prenatal care or prenatal clinical treatment for a pregnant subject, comprising:
(a) determining the gestational age (GA) of a pregnancy according to the method of any of embodiments 73-91; and
(b) selecting a prenatal care or prenatal clinical treatment for the pregnant female subject based on the GA of the pregnancy.
93. A method for performing a prenatal care or prenatal clinical treatment on a pregnant subject, comprising performing a prenatal care or prenatal clinical treatment on a pregnant female subject, wherein the prenatal care or prenatal clinical treatment is selected according to the method of embodiment 92.
94. The method of any of embodiments 71, 72, 92, or 93, wherein the prenatal care or prenatal clinical treatment is a medical abortion or early aspiration; a decision about a medical abortion regimen; a decision about the risk of embryotoxicity; a clinical examination; a vaccination; a risk assessment; a fetal assessment; a blood assay; a urine assay; vitamin supplementation; a test for disease; education; counseling; or any combination of any of the foregoing.
95. The method of any of embodiments 71, 72, 92, or 93, wherein the prenatal care or prenatal clinical treatment is a medical abortion or early aspiration.
96. The method of any of embodiments 1-95, wherein the obtained biological sample has a volume between or between about 0.5 μL and 10 μL, inclusive.
97. The method of any of embodiments 1-96, wherein the obtained biological sample has a volume between or between about 0.5 μL and 5 μL, inclusive, optionally wherein the obtained biological sample has a volume of or of about 1 μL.
98. The method of any of embodiments 1-97, wherein the test sample is prepared by diluting the obtained biological sample with a sample diluent prior to the measuring.
99. The method of any of embodiments 1-97, further comprising diluting the obtained biological sample with a sample diluent prior to the measuring.
100. The method of embodiment 98 or embodiment 99, wherein the volume of the obtained biological sample is diluted between or between about 2-fold and 100-fold, inclusive.
101. The method of any of embodiments 1-100, wherein the volume of the obtained biological sample is diluted between or between about 2-fold and 75-fold, inclusive, optionally wherein the volume of the obtained biological sample is diluted or diluted about 5-fold or 50-fold.
102. The method of any of embodiments 1-101, wherein the sample diluent comprises one or both of buffered saline and a nonionic detergent.
103. The method of any of embodiments 1-102, wherein the obtained biological sample is a whole blood sample.
104. The method of any of embodiments 1-102, wherein the obtained biological sample is a serum sample.
105. The method of any of embodiments 1-104, further comprising obtaining the biological sample from the pregnant female subject.
106. The method of any of embodiments 9-105, wherein the capture antibody and/or the detection antibody is capable of specifically binding to (1) homodimeric PAPP-A and (2) the heterotetrameric PAPP-A/proMBP complex equimolarly or about equimolarly.
107. The method of any of embodiments 9-106, wherein the capture antibody is unlabeled.
108. The method of any of embodiments 9-107, wherein the capture antibody is immobilized on a solid support.
109. The method of any of embodiments 5-108, wherein the solid support is a bead, column, array, assay plate, microwell, cartridge, stick, filter, or strip.
110. The method of any of embodiments 5-109, wherein the solid support is formed of glass, polysaccharides, polyacrylamides, polystyrene, polyvinyl alcohol, nitrocellulose, cellulose, nylon, and/or silicones.
111. The method of any of embodiments 11-110, wherein the detectable label is or comprises horseradish peroxidase.
112. The method of any of embodiments 1-111, wherein the method is carried out using a point-of-care device.
113. The method of any of embodiments 1-112, wherein the method is carried out using a lateral flow stick.
114. The method of any of embodiments 1-111, wherein the method is carried out in a laboratory.
115. The method of any of embodiments 1-114, wherein the method is carried out without an ultrasound.
116. The method of any of embodiments 23-73 and 94-115, further comprising comparing the classified GA to a GA as determined by performing an ultrasound.
117. A device for carrying out the method of any of embodiments 1-116.
118. The device of embodiment 117 that is a hand-held device or a point-of-care device.
119. The device of embodiment 116 or embodiment 117, wherein the device comprises a solid support and one or more antibody capable of independently specifically binding to (1) homodimeric PAPP-A and (2) a heterotetrameric PAPP-A/proMBP complex, wherein at least one of the one or more antibody is immobilized on the solid support.
120. The device of embodiment 119, wherein the solid support is a bead, column, array, assay plate, microwell, cartridge, stick, filter, or strip.
121. The device of embodiment 119 or embodiment 120, wherein the solid support is formed of glass, polysaccharides, polyacrylamides, polystyrene, polyvinyl alcohol, nitrocellulose, cellulose, nylon, and/or silicones.
122. The device of any of embodiments 117-121 that is a lateral flow device. 123. A kit for carrying out the method of any of embodiments 1-116, comprising a reference PAPP-A sample and the device of any of embodiments 117-122, wherein:
the concentration of PAPP-A in the reference PAPP-A sample is for a predetermined GA cutpoint; and
the PAPP-A in the reference PAPP-A sample comprises heterotetrameric PAPP-A/proMBP complex.
124. The kit of embodiment 123, wherein the PAPP-A in the reference PAPP-A sample further comprises homodimeric PAPP-A.
125. The kit of embodiment 123 or embodiment 124, wherein the PAPP-A in the reference PAPP-A sample consists essentially of heterotetrameric PAPP-A/proMBP complex.
126. The kit of any of embodiments 123-125, wherein the predetermined GA cutpoint is a timepoint between or between about 5 weeks and 40 weeks, inclusive.
127. The kit of embodiment 123-125, wherein the predetermined GA cutpoint is a timepoint between or between about 64 days and 140 days, inclusive.
128. The kit of embodiment 123-125, wherein the predetermined GA cutpoint is or is about 63 days, 70 days, or 77 days.
129. The kit of any of embodiments 123-128, wherein the concentration of reference PAPP-A in the reference PAPP-A sample is between or between about 150 ng/mL and 250 ng/mL, inclusive.
130. The kit of any of embodiments 123-128, wherein the concentration of reference PAPP-A in the reference PAPP-A sample is between or between about 160 ng/mL and 200 ng/mL, inclusive.
131. The kit of any of embodiments 123-128, wherein the concentration of reference PAPP-A in the reference PAPP-A sample is or is about 180.679 ng/mL.
132. The kit of any of embodiments 123-131, wherein the reference PAPP-A sample is a first reference PAPP-A sample and the predetermined GA cutpoint is a first predetermined GA cutpoint, and further comprising a second reference PAPP-A sample, wherein:
the concentration of PAPP-A in the second reference PAPP-A sample is for a second predetermined GA cutpoint that is later than the first predetermined GA cutpoint; and
   the PAPP-A in the second reference sample comprises (1) homodimeric PAPP-A and/or (2) the heterotetrameric PAPP-A/proMBP complex.
133. The kit of embodiment 132, wherein the second predetermined GA cutpoint is a timepoint between or between about 5 weeks and 40 weeks, inclusive.
134. The kit of embodiment 132, wherein the second predetermined GA cutpoint is a timepoint between or between about 64 days and 140 days, inclusive.
135. The kit of embodiment 132, wherein the second predetermined GA cutpoint is or is about 63 days, 70 days, or 77 days.
136. The kit of embodiment 132, wherein the second predetermined GA cutpoint is or is about 105 days.
137. The kit of embodiment 132, wherein the first predetermined GA cutpoint is or is about 63 days, and the second predetermined GA cutpoint is or is about 105 days+/−1 week, optionally about 98 days or 112 days.
138. The kit of embodiment 132, wherein the first predetermined GA cutpoint is or is about 70 days, and the second predetermined GA cutpoint is or is about 105 days+/−1 week, optionally about 98 days or 112 days.
139. The kit of embodiment 132, wherein the first predetermined GA cutpoint is or is about 77 days, and the second predetermined GA cutpoint is or is about 105 days+/−1 week, optionally about 98 days or 112 days.

### VI. Examples

The following examples are included for illustrative purposes only and are not intended to limit the scope of the invention.

### Example 1: Serum Collection from Pregnant Subjects at Various Gestational Ages

A study was conducted that included 245 female subjects with pregnancies within various Gestational Age (GA) ranges. Subjects were selected based on the following inclusion criteria: confirmed intrauterine pregnancy by ultrasound; no evidence of maternal or fetal conditions (pregnancy-induced hypertension (pre-eclampsia) or diabetes; not known to have a non-viable pregnancy, multiple pregnancy, a pregnancy with a fetal, chromosomal or other abnormality, or a pregnancy with a fetal growth restriction; not currently having substantial maternal bleeding such as vaginal bleeding, or other unusual pelvic pain or other symptom that may indicate a pregnancy complication; no anticoagulant use; and no use of assisted reproductive technology in current pregnancy. Subjects ranged in weight from 43-150 kilograms (median 66 kilograms) and had body mass indices ranging from 17 to 52 kg/m² (median 24 kg/m²). Additional characteristics are outlined in Table E1.

TABLE E1
Characteristics of Enrolled Subjects

| | N | (%) |
|---|---|---|
| Age (years) | | |
| ≤24 | 80 | 33% |
| 25-29 | 65 | 27% |
| 30-34 | 66 | 27% |
| ≥35 | 34 | 14% |
| Race | | |
| Hispanic | 7 | 3% |
| White | 87 | 36% |
| Black | 116 | 47% |
| Asian | 12 | 5% |
| Other | 10 | 4% |

TABLE E1
Characteristics of Enrolled Subjects

| | | |
|---|---|---|
| Multiple | 13 | 5% |

Maternal conditions

| | | |
|---|---|---|
| Current tobacco use | 25 | 10% |
| Alcohol use in this pregnancy | 33 | 13% |
| Used medications in the past | 98 | 40% |
| week | | |
| Medical conditions | 89 | 36% |

GA was determined from reported findings from the earliest ultrasound present in the patient's medical chart. Enrollment quotas were established to ensure representation across the GA spectrum, with oversampling of patients between 5 and 15 weeks (105 days) of gestation. Table E2 outlines the distribution of subjects across the GA spectrum for this group of assessed subjects.

TABLE E2
Gestational age at sample collection (days)

| Gestational | Number of | Percent of |
|---|---|---|
| age | subjects | subjects |
| 34-41 | 21 | 9% |
| 42-48 | 20 | 8% |
| 49-55 | 20 | 8% |
| 56-62 | 20 | 8% |
| 63-69 | 21 | 9% |
| 70-76 | 20 | 8% |
| 77-83 | 20 | 8% |
| 84-90 | 21 | 9% |
| 91-97 | 21 | 9% |
| 98-104 | 21 | 9% |
| 105-139 | 10 | 4% |
| 140-174 | 10 | 4% |
| 175-209 | 10 | 4% |
| 210-273 | 10 | 4% |

Serum specimens were obtained from all 245 women enrolled in the study. Blood was drawn from a vein into a non-heparinized tube, allowed to clot, and then centrifuged. All specimens were refrigerated and then frozen to −80° C. on the day of collection.

### Example 2: Immunoassays for PAPP-A During Pregnancy

To assay for serum PAPP-A concentrations, 120 of the maternal serum samples collected as described in Example 1 were analyzed. The selected samples included all 67 specimens available with GA between 58-83 days, spanning two weeks before and after a cut-point of 70 days (10 weeks). Additionally, 24 specimens with GA between 34-56 days and 32 specimens with GA between 85-231 days were also randomly selected for analysis. In total, 108 specimens with GA less than 105 days (15 weeks) were analyzed. Characteristics of the 120 pregnant women whose serum samples were analyzed are shown in Table E3. Sample aliquots were shipped on dry ice and maintained frozen at −80° C. until the immunoassays were performed. Individual samples were thawed only once, on the day of the assay unless otherwise specified.

TABLE E3
Characteristics of Pregnant Women with Analyzed Serum Samples

| Characteristic | Result | |
|---|---|---|
| Age, mean ± SD | 28.4 ± 5.4 | |
| Race/Ethnicity, n (%) | | |
| Non-Hispanic White | 42 | (35.0) |
| Non-Hispanic Black | 57 | (47.5) |
| Hispanic | 4 | (3.3) |
| Other or multiple races | 17 | (14.2) |
| Pre-pregnancy BMI, mean ± SD | 25.9 ± 6.37 | |
| Tobacco use in index pregnancy, n (%) | 13 | (10.8) |
| Alcohol use in index pregnancy, n (%) | 17 | (14.2) |
| Medical co-morbidities *, n (%) | 48 | (40) |
| Gestational age at sample | | |
| collection by trimester, n (%) | | |
| 1^{st} trimester (≤90 days) | 93 | (77.5) |
| 2^{nd} trimester (91-195 days) | 25 | (20.8) |
| 3^{rd} trimester (≥196 days) | 2 | (1.7) |
| Gestational age at sample | | |
| collection by periods relevant | | |
| to abortion options, n (%) | | |
| ≤70 days (≤10^{0/7} weeks) | 55 | (45.8) |
| 71-104 days (10^{1/7}-14^{6/7} weeks) | 53 | (44.2) |
| ≥105 days (≥15^{0/7} weeks) | 12 | (10) |

Abbreviations:
BMI, body mass index;
SD, standard deviation
* Includes anemia, nausea, arthritis, uterine anomalies, ovarian disorders, thyroid disease, genital infections, and respiratory, neurologic, orthopedic, gastrointestinal, skin, and psychiatric conditions.

After screening commercially available PAPP-A immunoassays, four assay kits were selected. These chosen assays differed in terms of type of capture antibody, detection antibody, standard curve range, requirement for initial sample volume, and traceable quality controls (TQC) included or not included in the assay kit. The vendor-determined characteristics for the four chosen PAPP-A immunoassays are presented in Table E4 and are hereafter referred to as 1-R&D Systems, 2-ThermoFisher, 3-Creative Diagnostics, and 4-Ansh Labs. In terms of reactivity, the 4-Ansh Labs immunoassay kit specifically indicates that the antibody pair measures homodimeric PAPP-A and a heterotetrameric complex containing PAPP-A and proMBP (2:2) approximately in equimolar concentration, but does not measure proMBP alone. This specificity for both PAPP-A proteoforms was confirmed by Western blot using the capture antibody of the 4-Ansh immunoassay. The three other assay kits did not note preferential binding for different PAPP-A proteoforms. Of the four assays, 3-Creative Diagnostics and 4-Ansh Labs included low- and high-level TQC, which in this study were tested reciprocally on all assays.

TABLE E4
Vendor-Determined Characteristics for PAPP-A Immunoassays

| Vendor | Assay | | |
|---|---|---|---|
| Catalog # | Format & | | |
| Lot # | Antibody | Standards and | Serum |
| Expiration Date | Considerations | Controls | Volume |
| R&D Systems | Capture: | Standard: | 50 μL of serum |
| Minneapolis, | anti-PAPP-A | lyophilized stock of | required and |
| Minnesota | monoclonal pre- | 500 ng/mL | added to plate |
| DPPA00 | coated on plate | Standard curve: | |
| Lot# P222407 | Detection: | 7-point | |
| Sep. 29, 2020 | polyclonal HRP- | 0.78; 1.56; 3.13; 6.25; | |
| | conjugated 2^{nd} | 12.5; 25; 50 ng/mL | |
| | antibody | (serial dilution | |
| | | factor 2) | |
| | | Recommended | |
| | | equation: | |
| | | log-log | |
| | | TQC: | |
| | | none | |
| ThermoFisher/ | Capture: | Standard: | 50 μL of serum |
| Invitrogen | anti-PAPP-A | lyophilized standard | required |
| EHPAPPA | monoclonal pre- | of 500 ng/mL | 100 μL of 2x |
| Lot# 53020320 | coated on plate | Standard curve: | diluted serum |
| February 2021 | Detection: | 7-point: | added to plate |
| | biotinylated 2^{nd} | 0.686; 2.058; 6.123; | |
| | antibody followed | 18.52, 55.56, 166.7, | |
| | by streptavidin-HRP | 500 ng/mL | |
| | | (serial dilution | |
| | | factor 3) | |
| | | Recommended | |
| | | equation: | |
| | | 4-parameter | |
| | | TQC: | |
| | | none | |
| Creative | Capture: | Standard: | 10 μL of serum |
| Diagnostics | anti-PAPP-A | 6 vials of lyophilized | required and |
| Shirley, New York | polyclonal pre- | calibrators | added to plate |
| DEIA2304 -96T | coated on plate | Standard curve: | |
| Lot# 46K109-2 | Detection: | 6-point lot specific | |
| Oct. 31, 2020 | polyclonal HRP- | (includes blank): 0; 1; | |
| | conjugated 2^{nd} | 2.5; 5.0; 15.0; 30.0 | |
| | antibody | μg/mL | |
| | | Recommended | |
| | | equation: | |
| | | 4-parameter | |
| | | TQC supplied (lot | |
| | | specific): | |
| | | High level: 19 μg/mL | |
| | | Low level: 4 μg/mL | |
| Ansh Labs | Capture: | Standard suppled: | 50 μL of 150x |
| Webster, Texas | anti-PAPP-A/ | 6 vials of lyophilized | diluted serum |
| AL-106 | proMBP complex | calibrators | added to plate |
| Lot# 031119 | monoclonal pre- | Standard curve: | |
| Nov. 27, 2020 | coated on plate | 6-point lot specific | |
| | Detection: | (includes blank) 0; | |
| | monoclonal HRP- | 26; 82; 323 1420; | |
| | conjugated 2^{nd} | 5004 ng/mL | |
| | antibody | Recommended | |
| | | equation: | |
| | | log-log with cubic | |
| | | regression | |
| | | TQC supplied (lot | |
| | | specific): | |
| | | High level: 682.5 ± | |
| | | 136.5 ng/mL | |
| | | Low level: 164 ± | |
| | | 41 ng/mL | |

For each of the tested assays, five kits from the same manufactured lot were purchased. Standards, samples, and TQCs were assayed in duplicate following the enclosed instruction manuals. All plates were read at 450 nm on a Versamax spectrophotometer (Molecular Devices, San Jose CA) equipped with Softmax Pro 5.4.1 software and wavelength correction at 570 or 630 nm as recommended. Samples with readings above the value of the highest standard were re-assayed at 10× dilution. This was necessary for 1-R&D Systems and 3-Creative Diagnostics immunoassays. Finally, a subset of 33 samples was re-assayed with the 4-Ansh Labs immunoassay changing the initial dilution from the recommended 150-fold to 5-, 50- or 1500-fold to optimize sample dilution for samples at the extremes of the GA range. For the 150-fold, 50-fold, and 5-fold dilutions, 0.33 μL, 1 μL, and 19 μL of serum, respectively, were used per well. All repeated analyses used a previously frozen and thawed aliquot.

To prepare specimens with PAPP-A concentrations corresponding to that of various GA ranges, three pooled samples were each generated by mixing 11 individual serum samples anticipated to have low, mid and high PAPP-A concentrations based on previously reported PAPP-A values and GA intervals. Samples in the low-level (LL), mid-level (ML), and high-level (HL) pools represented three GA ranges<70 days, between 71-91 days, and between 92-161 days, respectively. Pooled specimens were aliquoted to limit freeze and thaw cycles to twice, and each aliquot was assayed in triplicate on every plate for repeatability analyses.

In addition, the concentrations of proMBP and PAPP-A2, a metalloprotease with sequence similarity to PAPP-A, were measured in a subset of the serum specimens (n=61, GA range 34-163 days). The immunoassays used were commercially available immunoassays (proMBP ELISA: Creative Diagnostics Cat #DEIA-BJ186; and PRG2/MBP ELISA LSBio: Cat #LS-F5876) and designed to be specific for each respective analyte without interference from PAPP-A.

For statistical analyses, demographic data is presented as mean with standard deviation (SD) or as n with group percentage. Immunoassay results from duplicate wells were interpolated relative to a standard curve run on the same plate, averaged after subtraction of blank wells, and reported as the final result for each sample. A limit of blank (LOB) was calculated for each immunoassay type from the average of blank values on all plates+1.645×SD of blank values. The assay's limit of detection (LOD) was calculated as LOB+1.645×SD of LL pools on three different plates. Correlations between log-transformed immunoassay results and GA (in linear and log format) and between pairwise immunoassays were analyzed using Person product moment correlation. Parameter of receiver operator characteristic (ROC) analysis such as area under the ROC (AUROC) plot, sensitivity, specificity, and likelihood ratios (LR) using sonographically determined GA as comparator test were used as indicators of diagnostic accuracy. Optimal cut-offs for each immunoassay in the study population were determined based on the maximal Youden Index. This technique determines the most appropriate cut-off value, which corresponds to a point on the AUROC plot with the highest vertical distance from the 45° diagonal line (the AUROC of an uninformative test). Agreement between the immunoassay results dichotomized by their optimal cut-off points and GA dichotomized by the 70 days GA cut-off was measured using the Cohen's kappa. The degree of concordance was assigned based on the scale suggested by Landis and Koch (0.21-0.40 “fair”; 0.40-0.60 “moderate”, 0.61-0.80 “substantial”; 0.81-1.00 “almost perfect”). Pairwise comparison of the AUROC plots for immunoassay results as continuous variables or dichotomized by their own optimal cut-off was conducted using the non-parametric method of De Long. Confidence intervals were calculated using the bootstrapping method. Correlation between variables was analyzed using Pearson or Spearman correlation as noted. Agreement analysis was performed using the method of Passing and Bablok which is suitable for highly correlated data. Statistical analyses were performed with SigmaPlot (v14, Systat Software, San Jose, CA) and MedCalc (Broekstraat, Belgium) statistical software. TableCurve 2D (Systat Software) was used for curve fitting. A p-value<0.05 was considered significant throughout all analyses.

### A. Immunoassay Performance on Gestational Age Classification (70-Day Cutpoint)

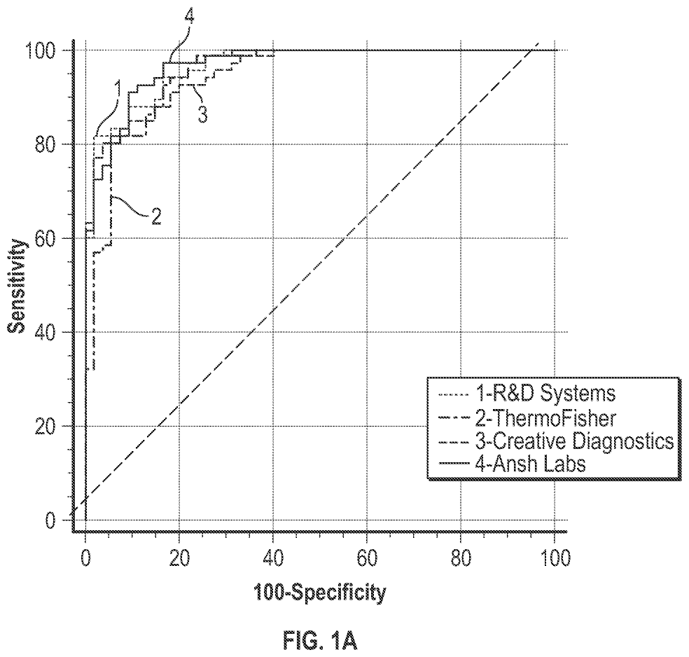

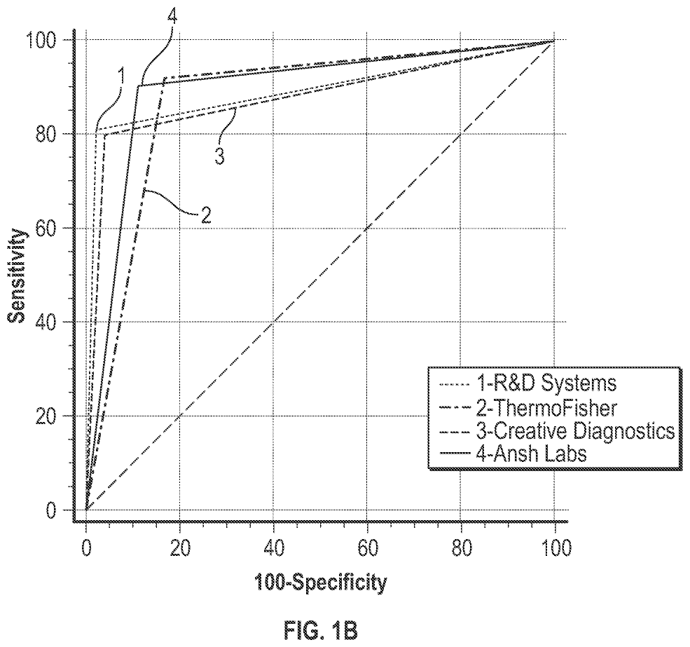

FIGS. 1A-1B shows the ROC plots for predicting GA>70 days for the four PAPP-A assays using the sonographically determined GA as the comparator test. The plots show the immunoassay results as continuous variables (FIG. 1A) or dichotomized above or below the optimal cut-off determined by each tests' Youden Index (FIG. 1B). The diagnostic accuracy parameters are presented in additional detail in Table E5. All four assays had sensitivities and specificities of at least 80%, and AUROC values ranged from 0.948 for 2-ThermoFisher assay to 0.968 for 4-Ansh Labs assay.

TABLE E5
Diagnostic Accuracy (GA > 70 days) of PAPP-A Immunoassays

| | AUROC | Youden | Associated | | Sensitivity | Specificity | +LR | −LR |
|---|---|---|---|---|---|---|---|---|
| Immunoassay | [95% CI] | Index J | Criterion | | [95% CI] | [95% CI] | [95% CI] | [95% CI] |
| 1- | 0.966 | 0.7972 | >6.033 | ng | 81.54 | 98.18 | 44.85 | 0.19 |
| R&D | [0.915- | | | | [70.0- | [90.3- | [6.4- | [0.1- |
| Systems | 0.990] | | | | 90.1] | 100.0] | 313.8] | 0.3] |
| 2- | 0.948 | 0.7594 | >13.988 | ng | 92.31 | 83.64 | 5.65 | 0.09 |
| ThermoFisher | [0.891- | | | | [83.0- | [71.2- | [3.1- | [0.04- |
| | 0.980] | | | | 97.5] | 92.2] | 10.31] | 0.2] |
| 3- | 0.955 | 0.7636 | >8,604.00 | ng | 80.00 | 96.36 | 22.00 | 0.21 |
| Creative | [0.901- | | | | [68.2- | [87.5- | [5.6- | [0.2- |
| Diagnostics | 0.985] | | | | 88.9] | 99.6] | 86.2] | 0.3] |
| 4- | 0.968 | 0.8168 | >180.697 | ng | 90.77 | 90.91 | 9.98 | 0.10 |
| Ansh Labs | [0.918- | | | | [81.0- | [80.0- | [4.4- | [0.05- |
| | 0.991] | | | | 96.5] | 97.0] | 23.1] | 0.2] |

Abbreviations: AUROC, area under the receiver operating characteristic;
CI, confidence interval;
LR, likelihood ratio

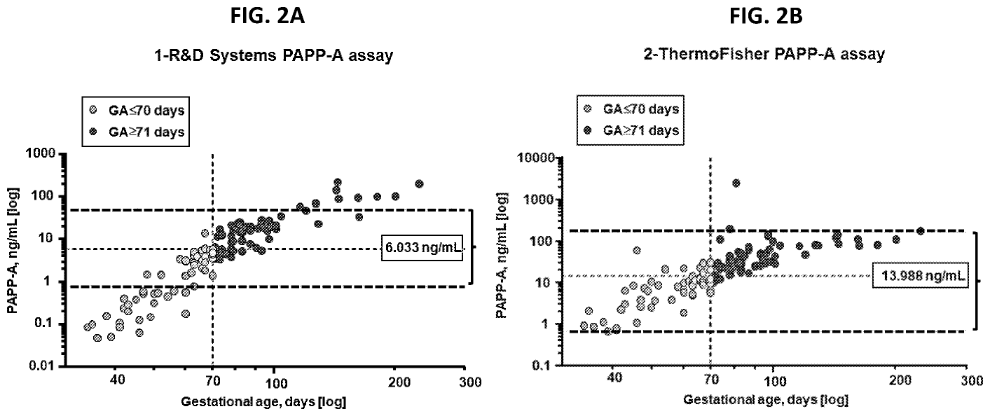

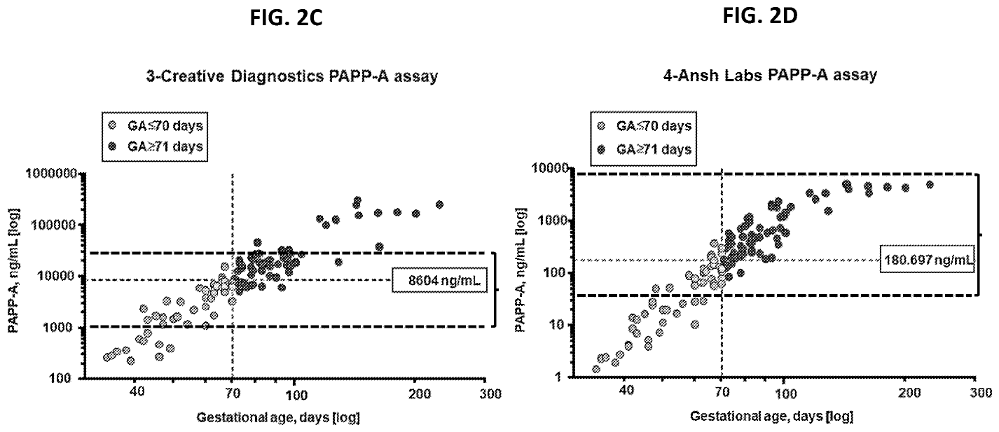

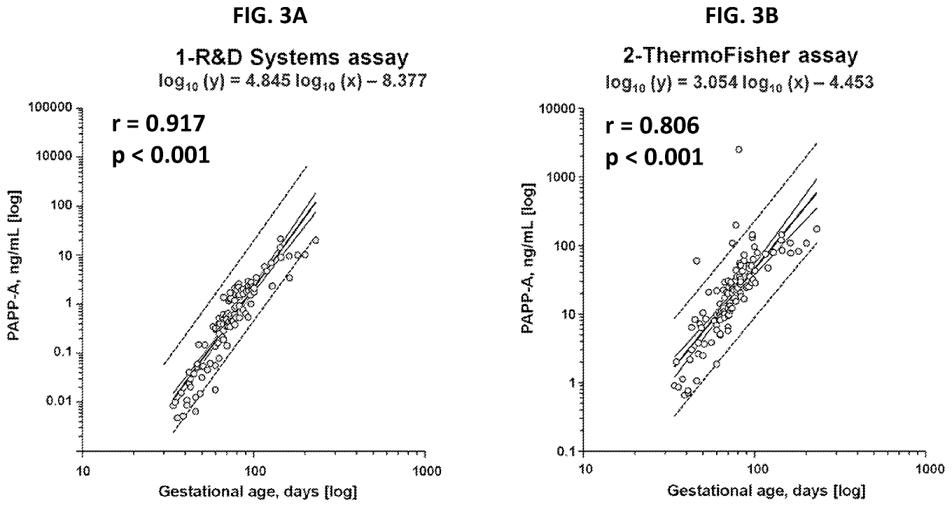

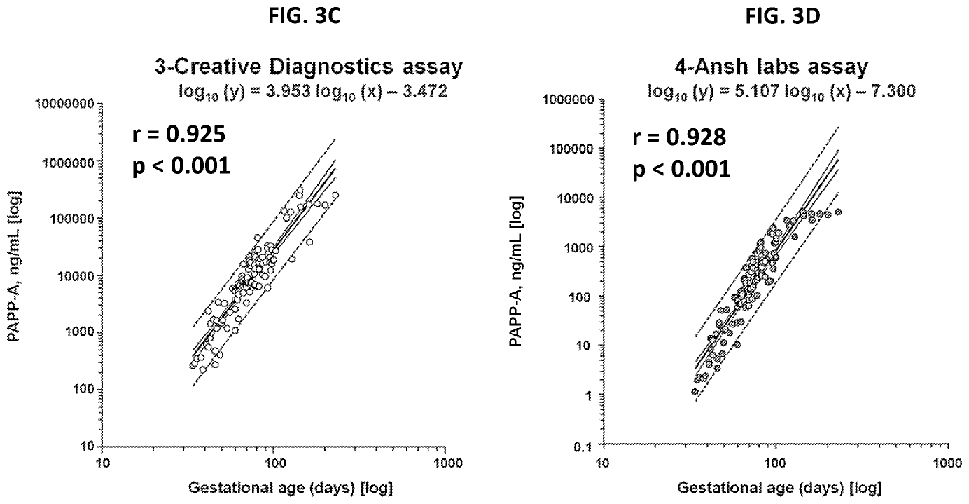

FIGS. 2A-2D shows the relationship between GA and PAPP-A immunoreactivity measured with the four different assays. As shown, all assays revealed a progressive logarithmic increase in PAPP-A as a function of log-transformed GA. The clinically relevant cut-off of 70 days is marked by the vertical dotted lines, and the samples with sonographically determined GA below or above this cut-off are differentially colored. The horizontal dotted lines mark the optimal cut-off based on maximal Youden Index determined through ROC analysis (Associated Criterion values shown in Table E5). The shaded area marks the respective assay's standard curve range. As shown, there was a clear separation of samples with all assays based on the 70 days cut-off despite the fact that the units of the y-axes differed by several orders of magnitude among the assays. For all assays, there was a significant linear correlation between the log-transformed immunoassay value and the log-transformed GA to a point where predictive equations could be derived for each assay, as shown in FIGS. 3A-3D. Pearson coefficients for these correlations ranged from 0.806 for 2-ThermoFisher to 0.928 for 4-Ansh Labs, with 1-R&D Systems and 3-Creative Diagnostics assays having Pearson coefficients of 0.917 and 0.925, respectively (p-value less than 0.001 for all assays). Slope and intercept values for fitted equations (log₁₀-transformed PAPP-A concentrations as a function of login-transformed GA) are shown in Table E6.

TABLE E6
Fitted Linear Equations for Log₁₀-Transformed PAPP-A Concentrations
as a Function of Log₁₀-Transformed Gestational Age

| Immunoassay | Slope | Intercept |
|---|---|---|
| 1-R&D Systems | 4.845 | −8.377 |
| 2-ThermoFisher | 3.054 | −4.453 |
| 3- Creative Diagnostics | 3.953 | −3.472 |
| 4-Ansh Labs | 5.107 | −7.300 |

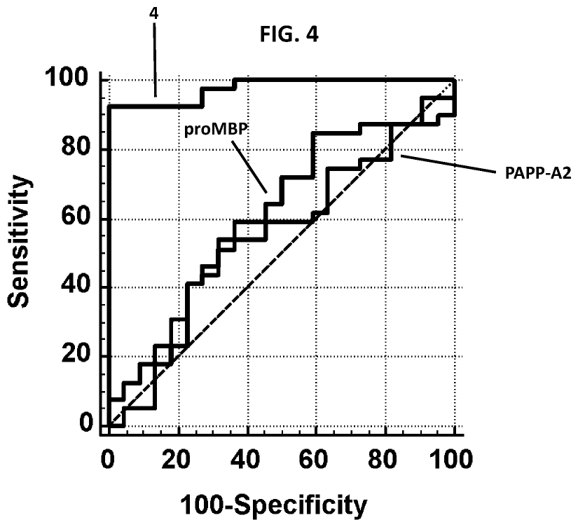

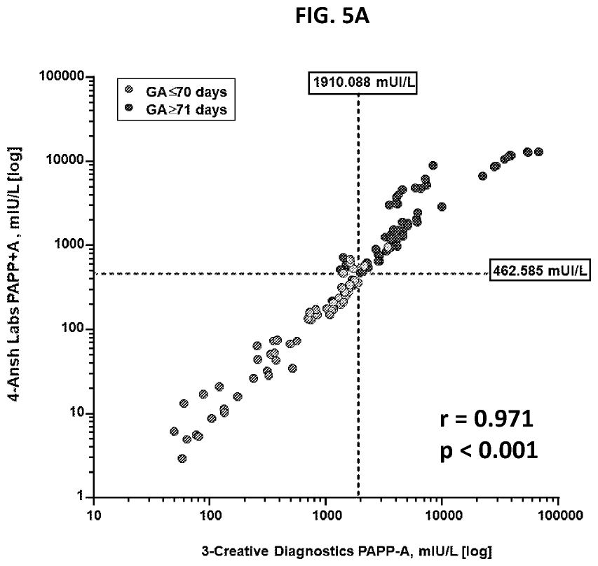

To determine whether the relationship of PAPP-A to GA extends to its binding partner proMBP or to PAPP-A2, a protein which shares ˜40% amino acid homology, these additional analytes were measured in a randomly selected subgroup of specimens (n=61; GA range: 34-163 days; 39 samples were from women with pregnancies≤70 days by ultrasound and 22 women were at GA≥71 days). No correlation between GA and serum proMBP either in linear or log format (r=−0.033, p=0.800) was observed, and only a weak correlation was observed between GA and PAPP-A2 (r=0.299, p=0.019), as opposed to the strong correlation of PAPP-A and GA in the same subset of samples (r=0.940, p<0.001). This was also apparent in comparative ROC analysis, as illustrated in FIG. 4. The AUROCs for proMBP (0.604 95% CI [0.470 to 0.727]) and PAPP-A2 (0.550 [0.417 to 0.678] were statistically not significant versus the 0.5 line (p=0.180 and p=0.520, respectively). By comparison, the AUROC for PAPP-A as measured using the 4-Ansh Labs assay in the same subset of samples was 0.977 [0.901 to 0.999] (p<0.001) with 100% [85-100] sensitivity and 92% [79-98] specificity.

### B. Measurement Variability Across Immunoassays

Differences in technical performance and results output were observed between the immunoassays. Table E7 shows a comparison of experimentally determined technical characteristics of the four assays when tested with individual patient samples, pooled specimens, TQC, and blank samples. Testing of the pooled specimens was informative to provide inter and intra-assay coefficients of variation between the plates. Coefficient of variations were all <10% except for 2-ThermoFisher. The concentration measured for each of the pooled samples was systematically higher than the average value from individual patient samples for 1-R&D systems, 2-ThermoFisher, and 3-Creative Diagnostics, but not for 4-Ansh Labs.

TABLE E7
Experimentally Determined Technical Characteristics of PAPP-A Immunoassays

| | | | | |
|---|---|---|---|---|
| | 1-R&D | 2- | 3-Creative | |
| Characteristic | Systems | ThermoFisher | Diagnostics | 4-Ansh Labs |

Analyses of individual patient samples

| | | | | |
|---|---|---|---|---|
| Number of high | 7 (6) † | 1 (1) † | 14 (12) † | 0 (0) |
| outliers at initial | | | | |
| analysis of | | | | |
| individual samples, | | | | |
| n (%) | | | | |
| Number of low | 24 (20) | 7 (6) | 12 (10) | 18 (15)* |
| outliers at initial | | | | |
| analysis of | | | | |
| individual samples, | | | | |
| n (%) | | | | |
| Measurements of | | | | |
| individual samples | | | | |
| used to prepare the | | | | |
| pooled contrived | | | | |
| samples | | | | |
| high level (HL), | 29.43 ± 7.83 | 158.30 ± 28.51 | 24,540.27 ± 7,546.80 | 1,826.57 ± 663.26 |
| mean ± SD | ng/mL | ng/mL | ng/mL | ng/mL |
| medium level (ML), | 5.99 ± 0.53 | 60.99 ± 3.72 | 6,793.41 ± 564.08 | 226.79 ± 31.29 |
| mean ± SD | ng/mL | ng/mL | ng/mL | ng/mL |
| low level (LL), | 1.17 ± 0.55 | 18.77 ± 14.40 | 2,519.41 ± 998.32 | 40.04 ± 18.49 |
| mean ± SD | ng/mL | ng/mL | ng/mL | ng/mL |

Analyses of pooled specimens

| | | | | |
|---|---|---|---|---|
| Measured pooled | | | | |
| samples | | | | |
| high level (HL), | 39.37 ± 3.10 | 164.49 ± 39.27 | 45,967.42 ± 5389.51 | 1,730.72 ± 159.93 |
| mean ± SD | ng/mL | ng/mL | ng/mL | ng/mL |
| medium level (ML), | 8.30 ± 0.36 | 54.96 ± 9.50 | 8,565.00 ± 881.58 | 222.50 ± 3.62 |
| mean ± SD | ng/mL | ng/mL | ng/mL | ng/mL |
| low level (LL), | 1.48 ± 0.14 | 18.92 ± 4.09 | 3,176.33 ± 249.20 | 37.16 ± 1.93 |
| mean ± SD | ng/mL | ng/mL | ng/mL | ng/mL |
| Intra-assay precision | 5.54% | 11.91% | 5.80% | 3.87% |
| (% CV) | | | | |
| Inter-assay precision | 7.13% | 20.92% | 9.95% | 5.35% |
| (% CV) | | | | |

Analyses of traceable quality control (TQC) samples

| | | | | |
|---|---|---|---|---|
| Measured TQC | | | | |
| from Creative | | | | |
| Diagnostics | | | | |
| high level (~19 | 33.53 ± 0.98 | 2,141.77 | 19,803.88 ± 496.50 | 1,153.93 ± 43.19 |
| μg/mL), mean ± SD | ng/mL | ng/mL | ng/mL | ng/mL |
| low level (~4 μg/mL), | 2.44 ± 0.059 | 790.32 | 4,455.75 ± 69.49 | 56.06 ± 1.71 |
| mean ± SD | ng/mL | ng/mL | ng/mL | ng/mL |
| Measured TQC | | | | |
| from Ansh Labs | | | | |
| high level (~682 | 0.13 | 7.36 ± 0.53 | 468.00 ± 444.06 | 726.25 ± 12.70 |
| ng/mL), mean ± SD | ng/mL‡ | ng/mL | ng/mL* | ng/mL |
| low level (~164 | 0.03 | 2.62 ± 0.08 | 424.00 ± 434.87 | 172.41 ± 4.75 |
| ng/mL), mean ± SD | ng/mL* | ng/mL* | ng/mL* | ng/mL |
| % CV in supplied | NA | NA | 2.80% | 3.77% |
| TQC | | | | |

Analyses of blank samples

| | | | | |
|---|---|---|---|---|
| Limit of detection | 0.46 | 3.83 | 2,826.10 | 15.09 |
| (LOD) | ng/mL | ng/mL | ng/mL | ng/mL |
| Limit of blank | 0.29 | 1.56 | 2,633.98 | 12.02 |
| (LOB) | ng/mL | ng/mL | ng/mL | ng/mL |

Abbreviations:
SD, standard deviation;
CV, coefficient of variation
*retested at 5x dilution (instead of the recommended 150x dilution);
† retested at 10x dilution;
‡values below the respective assay's limit of detection.
Limit of blank (LOB) was calculated as the mean of blank samples from all plates + 1.645 × standard deviation of the blanks
Limit of detection (LOD) was calculated as LOB + 1.645 × standard deviation of the measured low level pooled samples

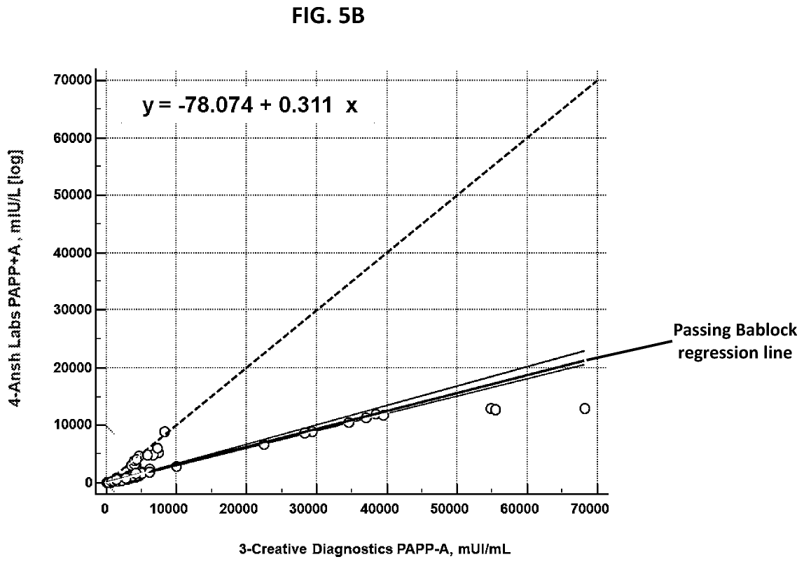

Both 3-Creative Diagnostics and 4-Ansh Labs immunoassays provided conversion formulas to mIU/L (3-Creative Diagnostics: 1 mIU/mL=4.5 mg/mL; 4-Ansh Labs: 1 ng/mL=2.56 μIU/mL). Each formula was applied in an effort to synchronize the readouts for the two assays. FIG. 5A presents the cross-correlation of PAPP-A concentration measured with the two assays when values were converted to mIU/L. As shown in Table E5, the optimal cut-off with 3-Creative Diagnostics assay (8,604 ng/mL) was 48-times higher than the value of the cut-off with 4-Ansh Labs (180.697 ng/mL). After applying the conversion formula and reprocessing the ROC analysis, the optimal cut-offs came closer together both as determined both by the Youden index (3-Creative Diagnostics: 1,910.088 mIU/L and 4-Ansh Labs: 462.585 mIU/L) and by the cut-offs resulting in 100% sensitivity (1,127.538 mIU/L and 212.008 mIU/L, respectively). While there was a high correlation in the log-log space between the results of the two assays (Pearson r=0.971, p<0.001), the Passing-Bablok agreement analysis on the mIU/L converted values suggests they are still not interchangeable. The confidence intervals for the systematic (intercept=−78.074, 95% CI [−102.792 to −57.481]) and proportional (slope=0.311, 95% CI [0.303 to 0.340]) differences did not span zero and significant deviation from linearity was noted (Cusum test p<0.001) (FIG. 5B).

### C. Effects of Sample Dilution on Immunoassay Readouts

As indicated in Table E7, Assays 1-R&D Systems and 3-Creative Diagnostics required a 10× sample dilution to interpolate the data for some samples with high GA. For low outliers, only the dilution on the 4-Ansh Labs assay, which had an initial recommended serum dilution of 150×, could be optimized. Four samples, all <40 days GA (34, 35, 36, and 38 days GA), had an undetectable low reading, and their values (1.13, 1.923, 2.175, and 2.091 ng/mL) were interpolated at 5× dilution using the 4-Ansh Labs assay. Additionally, 2-ThermoFisher had spurious high readings for several samples, which could not be corrected with optimization of the assay method. Additional experiments where secondary antibody was omitted indicated that those spurious high readings were not caused by endogenous biotin.

### D. Reference Standards for Immunoassays

Only the TQC samples from 3-Creative Diagnostics were readable on all four assays. The TQCs from 4-Ansh Labs were below LOB when tested on all three other assays. With the information from the TQC samples and the equations shown in FIGS. 3A-3D, the “theoretical GA” for the high and low TQC samples from 3-Creative Diagnostics when read on each of the assays was determined as follows: 111 and 64 days on 1-R&D Systems; 355 and 255 days on 2-ThermoFisher; 92 and 63 days on 3-Creative Diagnostics; and 107 and 66 days on 4-Ansh Labs. With the exception of that of 2-ThermoFisher, these values bracket the GA cut-point of 70 days. To create a reference PAPP-A sample with PAPP-A concentration corresponding to the GA cut-point of 70 days, 2 μL of the 3-Creative Diagnostics high TQC sample was mixed with 8 μL of the 3-Creative Diagnostics low TQC sample.

Additionally, the TQCs from 3-Creative Diagnostics were readable by the proMBP ELISA above the LOD (0.377 and 42.537 ng/ml respectively for low and high TQC), indicating that these TQCs contained the PAPP-A/proMBP complex. In contrast, proMBP was non-detectable in the TQCs from 4-Ansh Labs.

### E. PAPP-A Thresholds for Additional Gestational Age Cutpoints

Using the 4-Ansh Labs assay, PAPP-A serum concentration thresholds by which GA could be accurately classified as above or below various GA cutpoints throughout gestation were determined. Two exemplary thresholds were determined per GA cutpoint: the threshold yielding 100% sensitivity in classifying GA, and the threshold as chosen based on the Youden Index. These thresholds are shown in Table E8. Thresholds are reported in units of ng/mL as well as in units of mIU/mL.

TABLE E8
PAPP-A Thresholds for Various Gestational Age (GA) Cutpoints

| | 4-Ansh Labs, ng/mL | | 4-Ansh Labs, mIU/mL | |
|---|---|---|---|---|
| | Threshold for 100% | | Threshold for 100% | |
| | Sensitivity (Threshold | | Sensitivity (Threshold | |
| GA Cutpoint | Based on Youden Index) | | Based on Youden Index) | |
| >56 days | 8.197 | (52.346) | 20.984 | (134.006) |
| >63 days | 52.346 | (123.733) | 134.006 | (316.757) |
| >70 days | 82.816 | (180.697) | 212.008 | (462.585) |
| >77 days | 101.351 | (206.252) | 259.459 | (528.005) |
| >84 days | 182.514 | (463.890) | 467.236 | (1187.559) |
| >98 days | 574.025 | (1215.391) | 1469.504 | (3111.4) |
| >105 days | 1465.904 | (1465.904)† | 3752.713 | (3752.713)† |
| >112 days | 1465.904 | (1465.904)† | 3752.713 | (3752.713)† |
| >168 days | 4121.925 | (4121.925)† | 10552.129 | (10552.129)† |

†Among analyzed samples, the Youden index-based threshold yielded 100% sensitivity for that GA cutpoint

### F. Conclusions

Taken together, these results indicate that serum PAPP-A concentrations can be precisely determined using an immunoassay (4-Ansh Labs) that leverages an antibody capable of detecting homodimeric PAPP-A and heterotetrameric PAPP-A/proMBP complex approximately in equimolar concentration. As shown here, this immunoassay can be used to precisely measure serum PAPP-A concentration throughout pregnancy using <1 μL of serum sample, including for pregnant subjects with GA<105 days (15 weeks). In addition, dilution of said sample can be optimized, for instance decreased, in order to measure low PAPP-A concentrations in samples from pregnant subjects with low GA (<40 days). Moreover, serum PAPP-A concentrations measured using this immunoassay strongly correlated with ultrasound-determined GA and can be used to predict GA>70 days with high sensitivity and specificity. All together, these results suggesting that this immunoassay is best suited for use in a point-of-care test that assesses serum PAPP-A as a biomarker of GA early in pregnancy, for instance a point-of-care test designed to classify subjects as having GAs above or below the 70-day (10-week) cutpoint for medical abortion eligibility.

These results also indicate that such a point-of-care test could be used in conjunction with one or more reference PAPP-A samples containing the PAPP-A/proMBP complex as TQCs. As shown, different immunoassays can generate significantly different absolute PAPP-A concentrations for the same sample in the same testing conditions, and a conversion formula to mIU/mL may not be sufficient to obtain equivalence across assays. Thus, such reference PAPP-A samples can be used not only to improve equivalence across assays, but also to bracket or match PAPP-A concentration levels corresponding to GA cutpoints of interest.

### Example 3: Gestational Age Estimation Using PAPP-A in an Intended Use Population

A study was conducted to assess using serum PAPP-A concentration levels to estimate gestational age (GA) in patients seeking a medical abortion (MA). Members of this intended-use population were recruited at three clinics in Chicago, IL; New York, NY; and West Palm Beach, FL. Patients who were pregnant and presenting for abortion services at the study sites were eligible provided they had not yet had any transcervical or uterine procedures, had not yet used any abortifacient drugs (including for cervical preparation), had an ultrasound, were willing to provide a blood sample, and had not previously been enrolled in this study. Participants were interviewed to ascertain their pregnancy history, demographics, personal habits, health conditions, and current medications. Their charts were also reviewed to obtain their abortion outcome and any medical conditions or pregnancy complications. GA was assessed by using the reported findings of the earliest ultrasound documented in the medical chart. Pregnancies of unknown location (PUL) were recoded as 29 days, which represented 1 day below the lowest date reported in the sample.

Prior to any uterine instrumentation or intake of any abortifacient drugs, participants provided a blood sample. Blood samples were drawn by venipuncture into a serum-separator tube, allowed to clot, and then centrifuged. Serum was pipetted off and transferred to a screw cap transport tube (Micronic), packaged in a biobag wrapped in a gel pack inside a sealed bag (Mylar), and then placed in a small cardboard box in a FedEx Clinical pack. Specimens were registered using an electronic laboratory information management system portal, shipped overnight to a first laboratory location, aliquoted upon arrival in 0.3 mL aliquots, and frozen to −80° C. until PAPP-A was measured. Some frozen aliquots were shipped on dry ice to a second laboratory location, where they were also maintained at −80° C.

A total of 286 participants seeking abortion services were enrolled. Ten participants were disqualified because of blood sample collection problems, and an additional seven participants were excluded on clinical grounds. Five additional samples were excluded from the final analysis for heavy sample precipitation upon thawing. Therefore, the final study population with serum samples available for analysis consisted of 264 people.

The median age of the participants was 26 years (range 18-47], and 15% (39/264) were primigravidas. One-fifth (54/246) of the sample reported some cigarette-use, and 45% (122/246) reported alcohol-use during the index pregnancy. The study population included 8 multiple pregnancies, 4 PULs, and 2 cases of embryonic/fetal demise. The majority of the participants (64%, 170/264) had no maternal health conditions. At the time of enrollment, no participants had a known history of diabetes, incomplete or inevitable abortions, chromosomal or other structural fetal anomalies, molar pregnancies, abnormal placentation, fetal growth restriction, or large for gestational age. The array of self-reported and chart-abstracted conditions and medications used in the week prior to enrollment is shown in Table E9. There were no serious adverse events among study participants.

TABLE E9
Characteristics of Final Study Population

| Variable | Study population (n = 264) | |
|---|---|---|
| Age, years, median [range] | 26 | [18-47] |
| Age group, n (%) | | |
| ≤24 years | 97 | (37) |
| 25-29 years | 97 | (37) |
| 30-34 years | 40 | (15) |
| ≥35 years | 30 | (11) |
| Pre-pregnancy weight, pounds, median | 155 | [95-323] |
| [range] | | |
| BMI, mean [range] | 27.84 | [15.6-50.6] |
| Race and ethnicity, n (%) | | |
| Non-Hispanic Black | 140 | (53) |
| Non-Hispanic White | 44 | (17) |
| Hispanic | 38 | (14) |
| Asian | 2 | (1) |
| Other | 5 | (2) |
| More than one race | 35 | (13) |
| Gravidity, n (%) | | |
| Primigravida | 39 | (15) |
| Gravida 2 or 3 | 102 | (39) |
| Gravida 4 or more | 123 | (47) |
| Habits, n (%) | | |
| Current tobacco use | 54 | (20) |
| Alcohol use in this pregnancy | 119 | (45) |
| Maternal medical conditions, n (%) * | | |
| None | 170 | (64) |
| Asthma | 29 | (11) |
| Anemia † | 24 | (9) |
| Mental health issues ‡ | 15 | (6) |
| Fibroids or uterine anomalies | 13 | (5) |
| Multiple pregnancy | 8 | (3) |
| Hypertensive Disorder | 7 | (3) |
| Thyroid problems | 5 | (2) |
| Pregnancy of unknown location | 4 | (2) |
| Embryonic/fetal demise | 2 | (1) |
| Other § | 26 | (10) |
| Medications used in the past week, n (%) * | | |
| None | 159 | (59) |
| Acetaminophen | 26 | (10) |
| Vitamins and supplements ∥ | 21 | (8) |
| NSAIDS | 14 | (5) |
| Antihistamines | 12 | (5) |
| Antibiotics | 10 | (4) |
| Antiemetics | 10 | (4) |
| Bronchodilators | 9 | (3) |
| Antidepressants | 6 | (2) |
| Hormones | 5 | (2) |
| Other ¶ | 35 | (13) |

* includes both self-reported and abstracted from the chart
† includes self-reported and charted history of anemia
‡ includes anxiety, depression, post-traumatic stress disorder, bipolar, attention deficit disorder and schizoaffective disorder.
§ includes alcoholism, seasonal allergies, bacterial vaginosis, chronic bronchitis, eczema, endometriosis, epilepsy, GERD, hematometra, HSV II, hypocalcemia, impetigo, kidney infection, kidney stones, lupus, migraines, ovarian cysts, pneumonia, UTI, history of renal stents
∥ includes multivitamins, prenatal vitamins, and/or supplements
¶ other medications reported taken by fewer than 5 participants include: ACE inhibitors, antacids, anticonvulsants, antipsychotics, antifungals, antiparasitics, antitussives, antivirals, anxiolytics, beta blockers, decongestants, leukotriene receptor antagonist, diuretics, expectorants, laxatives, narcotics, proton-pump inhibitors, sedatives, stimulants, unspecified cold medicines or unspecified inhalers.

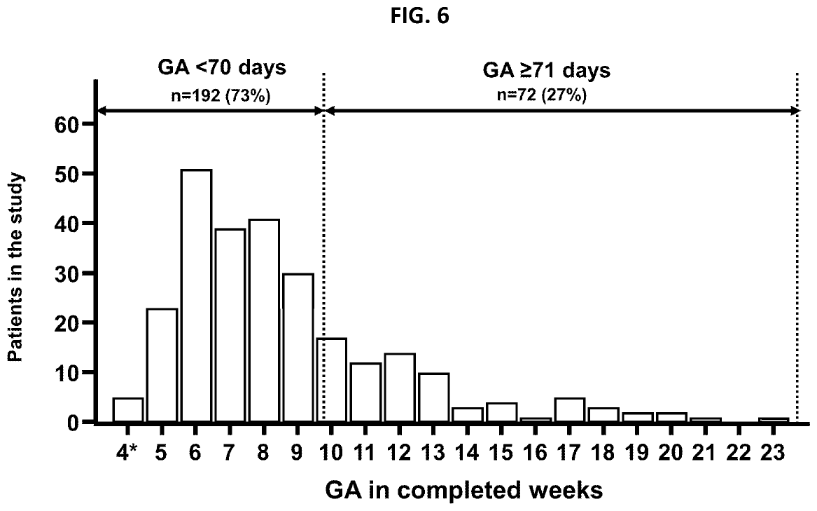

Patients had an ultrasound-determined GA ranging from 29 to 161 days with a median GA of 63 days. The distribution of GA by week is shown in FIG. 6. In total, 73% (192/264) of the participants had a GA≤71 days, for a prevalence of a GA above the current FDA-approved limit for MA of 27% (72/264).

Serum PAPP-A immunoassays were performed at both laboratory locations from aliquots that had not been previously thawed. The maximum storage time was less than 2 years. Two immunoassays were performed per sample. First, the 4-Ansh Labs immunoassay described in Example 2 was performed, first with a 150× serum dilution as specified in the manufacturer's instructions. Samples measuring below the lowest standard were re-measured with a 5× initial dilution, the 5× initial dilution as determined based on the results described in Example 2. Data was interpolated using a 3-order polynomial equation against a standard curve ranging from 26 to 5,004 ng/mL.

For comparison, serum PAPP-A was also measured with the 1-R&D Systems immunoassay kit described in Example 2. For this assay, 50 μL of undiluted serum was applied to the assay plate, and the procedure followed manufacturer instructions without modifications, except that samples measuring above the highest standard were re-assayed with 10× dilution. A log-log equation was used for interpolation of the 1-R&D Systems assay readouts against a standard curve ranging from 0.78 to 50 ng/mL.

Both immunoassays were conducted on technical duplicates whose values were averaged. As positive control samples, the low- and high-level TQC samples from the 3-Creative Diagnostics assay, described in Example 2, were applied on each plate to allow for equivalence calculations between 4-Ansh Labs and 1-R&D Systems assay measurements.

Statistical analyses were performed with SPSS (ver. 25, Chicago, IL) and MedCalc (Broekstraat, Belgium). ROC plots were used to determine the optimal cut-points for the intended-use population based on the Youden Index and the value that provided 100% sensitivity for detecting pregnancies of GA≥71 days. Misclassified patients were individually reviewed for clinical and demographic characteristics to identify possible sources of error. The 100% sensitivity value was selected with the idea in mind that there may be little clinical tolerance for false-negative tests. Confidence intervals for the area under the ROC curve (AUC) were calculated by the methodology of DeLong et al. (Biometrics 1988, 44(3):837-845). Comparison of AUROC derived from the 1-R&D Systems and 4-Ansh Lab assays was performed after the method of Hanley and McNeil (Radiology 1983, 148(3):839-843). The Fagan nomogram (N Engl J Med. 1975, 293(5):257-257) was used to visualize the post-test probability that an individual presenting at an abortion clinic analogous to the ones in this study has a GA≥71 days, given the observed test result with the 4-Ansh Labs PAPP-A assay.

In addition, contingency tables were used to validate the sensitivity, specificity, positive and negative predictive values (PPV and NPV), and likelihood ratios (LR) of the PAPP-A cut-points described in Example 2.

Relationships between GA and PAPP-A serum concentration levels as continuous variables in the intended-use population were explored in scatterplots after log-log transformation, and regression equations were derived. Solving the regression equation for each observed PAPP-A concentration resulted in the calculation of a PAPP-A-determined “biochemical GA” which was further analyzed for agreement with the ultrasound-determined GA using Lin's concordance correlation coefficient.

### A. Accuracy of PAPP-A as a Biomarker for Medical Abortion Eligibility

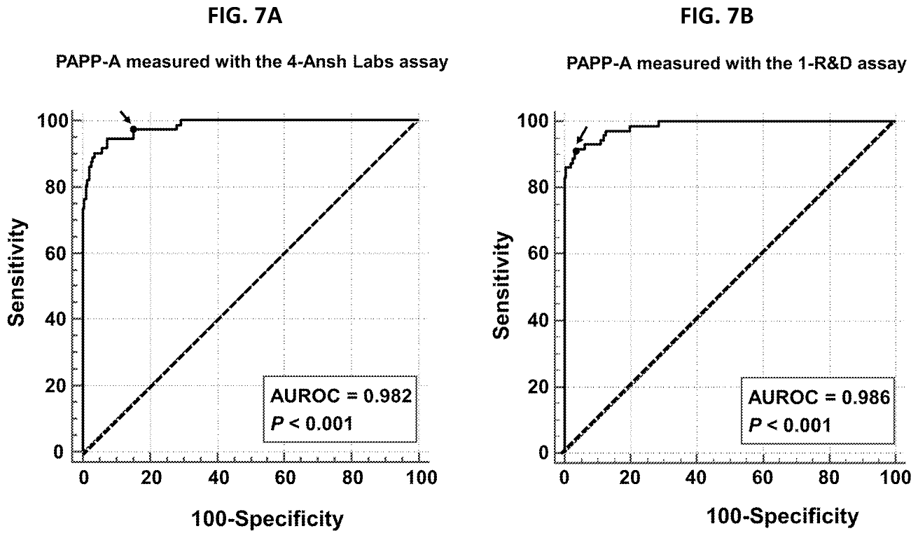

For the intended-use population, the 4-Ansh Labs assay produced an area under the receiver operating curve (AUC) of 0.982 [95% CI 0.958-0.994] (p<0.001, FIG. 7A), and the 1-R&D Systems assay produced an AUC of 0.986 [95% CI 0.963-0.996] (p<0.001, FIG. 7B), with no statistical significance between the two (p=0.220). The accuracy characteristics at the cut-off point corresponding to the Youden Index (optimizing sensitivity for maximal specificity) and the cut-off point corresponding to 100% sensitivity are listed in Table E10. Characteristics of the cut-off points from the study described in Example 2 as applied to the intended-use population are also shown in Table E10. Bracketed ranges in Table E10 refer to 95% confidence intervals.

TABLE E10
Pre-Specified and Sample-Derived Concentration Threshold
Performance for Predicting GA ≥ 71 days

| | | Youden | Conc. | | | | | PPV | NPV |
|---|---|---|---|---|---|---|---|---|---|
| | AUC | Index J | Threshold | Sensitivity | Specificity | +LR | −LR | (%) | (%) |
| 1- | 0.986 | 0.8854 | 6.033 | 73.6% | 100% | — | 0.26 | 100 | 91.1 |
| R&D | [0.963- | | ng/mL** | [61.9- | [98.1- | | [0.18- | | [87.4- |
| Systems | 0.996] | | | 83.3] | 100] | | 0.39] | | 93.8] |
| | | | 0.741 | 100% | 71.4 | 3.49 | 0 | 56.4 | 100 |
| | | | ng/mL | [95.0- | [64.4- | [2.79- | | [50.8- | |
| | | | **** | 100] | 77.6] | 4.36] | | 61.7] | |
| | | | 3.426 | 91.7% | 96.9% | 29.33 | 0.086 | 91.6 | 96.9 |
| | | | ng/mL | [82.7- | [93.3- | [13.30- | [0.040- | [83.1- | [93.6- |
| | | | ***** | 96.9] | 98.8] | 64.67] | 0.19] | 96.0] | 98.5] |
| 4- | 0.982 | 0.8715 | 82.816 | 97.2% | 84.4% | 6.22 | 0.033 | 69.7 | 98.8 |
| Ansh | [0.958- | | ng/mL*** | [90.3- | [78.5- | [4.47- | [0.008- | [62.3- | [95.4- |
| Labs | 0.994] | | | 99.7] | 89.2] | 8.66] | 0.13] | 76.2] | 99.7] |
| | | | 180.697 | 90.3% | 95.8% | 21.67 | 0.10 | 88.9 | 96.4 |
| | | | ng/mL** | [81.0- | [92.0- | [10.95- | [0.050- | [80.2- | [92.9- |
| | | | | 96.0] | 98.2] | 42.88] | 0.21] | 94.1] | 98.2] |
| | | | 44.153 | 100% | 70.8% | 3.43 | 0 | 55.9 | 100 |
| | | | ng/mL | [95.0- | [63.9- | [2.75- | | [50.4- | |
| | | | **** | 100] | 77.2] | 4.27] | | 61.3] | |
| | | | 133.2096 | 94.4% | 92.7% | 12.95 | 0.060 | 82.7 | 97.8 |
| | | | ng/mL | [86.4- | [88.1- | [7.80- | [0.023- | [74.3- | [94.6- |
| | | | ***** | 98.5] | 96.0] | 21.52] | 0.16] | 88.8] | 99.2] |

Abbreviations: AUC, area under the receiving operator curve;
CI, Confidence Interval;
LR, likelihood ratio;
PPV, positive predictive value;
NPV, negative predictive value
**concentration threshold based on Youden index in Example 2
***concentration threshold based on 100% sensitivity in Example 2
**** concentration threshold that corresponds to 100% sensitivity for sample in Example 3
***** concentration threshold that corresponds to the Youden index for sample in Example 3

The 4-Ansh Labs assay cut-off point that corresponded to the Youden Index in the intended use population was 133.210 ng/mL and resulted in a sensitivity of 94.4%, a specificity of 92.7%, a PPV of 82.7% and a NPV of 97.8% for an overall accuracy of 92.8%. Using this criterion, 18 specimens (7% of the sample) were misclassified with 14 false positives (PAPP-A value indicating a GA≥71 days but an ultrasound measured GA below that) and 4 false negatives. These false negatives were all in specimens with ultrasound determined GA≤78 days. Selecting a lower cut-off point of 44.153 mg/mL eliminated all of the false negative results and resulted in a specificity of 70.8%.

For the 1-R&D Systems assay, the cut-off point informed by the Youden index was 3.426 ng/mL, which resulted in a sensitivity of 91.7%, a specificity of 96.9%, a PPV of 91.7% and a NPV of 96.9%, for an overall accuracy of 95.5%.

### B. Comparison of PAPP-A Immunoassays for Gestational Age in an Intended Use Population

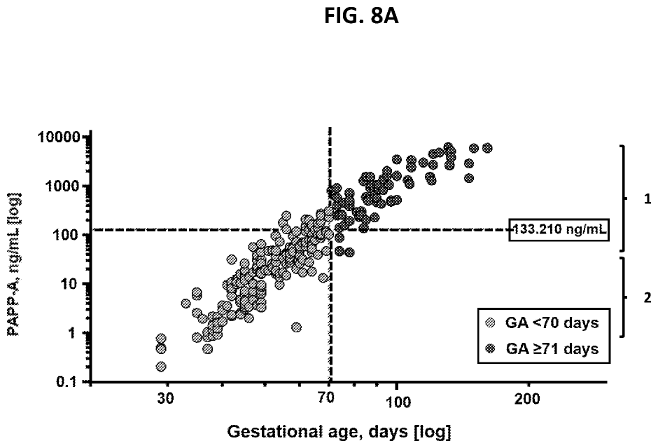

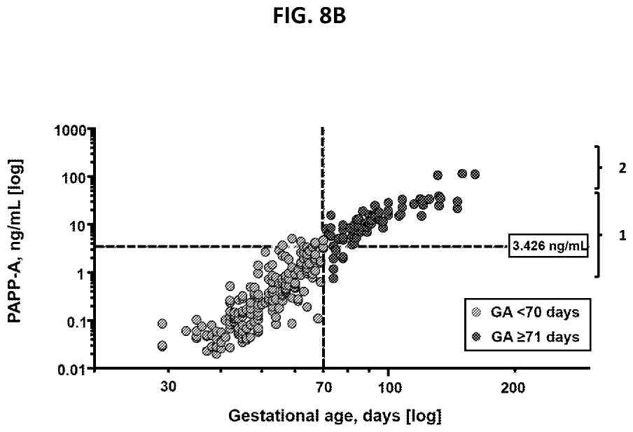

For the intended use population, FIGS. 8A-8B illustrate the log-log scatterplots of PAPP-A immunoreactivity measured with either the 4-Ansh Labs (FIG. 8A) or with the 1-R&D Systems assay (FIG. 8B) overlaid on the dynamic range of each assay (interval between lowest and highest standard) using the serum dilution recommended by the manufacturer or a modified sample dilution. The clinically relevant cut-off of 70 days is marked by the vertical dotted lines, and the samples with ultrasound-determined GA below or above this cut-off are differentially colored. The horizontal dotted lines mark the optimal cut-off based on maximal Youden Index determined through ROC analysis (Concentration Threshold values marked ***** in Table E10).

With the initial 150× serum dilution on the 4-Ansh Labs assay, 116 (44%) samples measured outside the limit of interpolation below the lowest standard. Of these, 109 samples (41% of total) were brought within the range of interpolation when the initial dilution was decreased to 5×. Seven samples (3% of total) remained below the value of the lowest standard. These included the 4 PUL cases along with another 3 cases below 40 days GA by ultrasound. At the 150× initial sample dilution, there were only 4 high outliers which could have been re-assayed at a higher dilution to improve linearity at higher GA, but which were nevertheless unambiguously classified as ≥71 days GA.

For the 1-R&D Systems assay, which requires 50 μL undiluted serum per well, only 46% (121/246) of values were within the range of quantification. These samples had a median GA of 74 days. There were 3 high outliers which were brought in the range of the assay at an additional 10× dilution.

### C. Calculation of Biochemical Gestational Age from PAPP-A Immunoassays

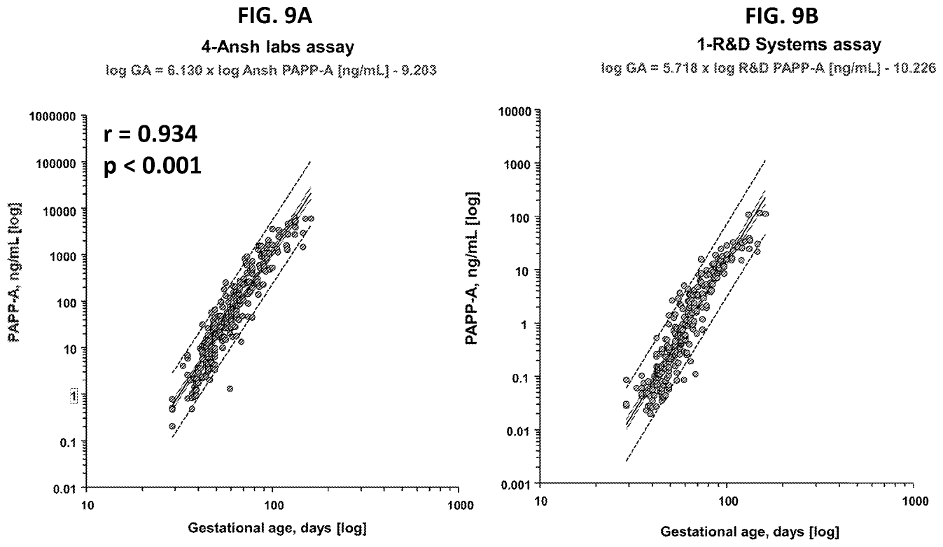

First-order log-log regression equations were determined for calculating biochemical GA based on serum PAPP-A concentration levels for the intended use population. Coefficients for these equations, along with the confidence and prediction intervals, are shown in FIGS. 9A-9B for the results of the 4-Ansh Labs (FIG. 9A) and for the 1-R&D Systems assay (FIG. 9B). The Pearson coefficient for 4-Ansh Labs was 0.934 (p<0.001) and for the 1-R&D Systems assay 0.926 (p<0.001). Slope and intercept values for the fitted equations (log₁₀-transformed PAPP-A concentrations as a function of log₁₀-transformed GA) are shown in Table E11.

TABLE E11
Fitted Linear Equations for Log₁₀-Transformed PAPP-A Concentrations
as a Function of Log₁₀-Transformed Gestational Age

| Immunoassay | Slope | Intercept |
|---|---|---|
| 1-R&D Systems | 5.718 | −10.226 |
| 4-Ansh Labs | 6.130 | −9.203 |

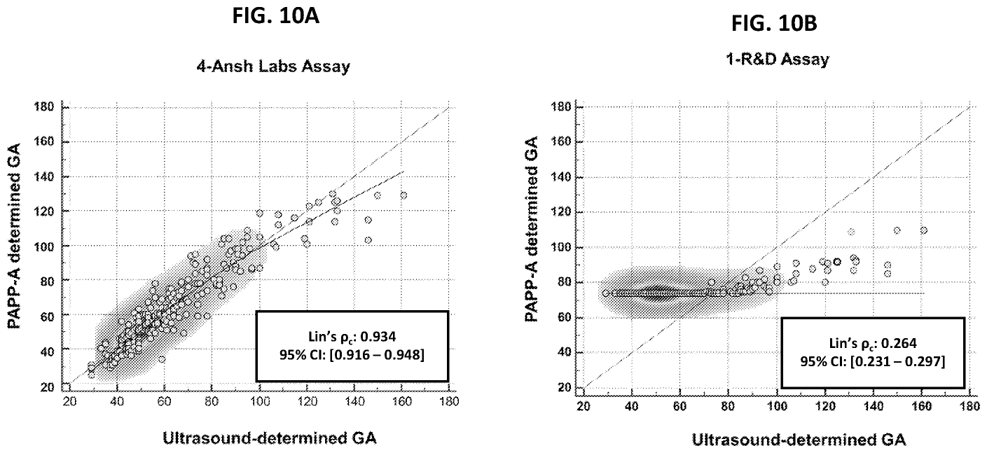

Solving the equations for each case resulted in a median [range] GA for the population of 58 [25-130] days for the 4-Ansh Labs assay and 74 [74-110] days for the 1-R&D Systems assay. With the 1-R&D Systems assay, no values lower than 74 days were calculated, as opposed to the 4-Ansh Labs assay, where there was significant variation on biochemical GA below 74 days. This difference can be visualized in the agreement scatterplots shown in FIGS. 10A-10B and quantitatively through the Lin's concordance coefficient, which was 0.934 [95% CI: 0.916-0.948] for the 4-Ansh Labs assay but only 0.264 [95% CI: 0.231-0.297] for the 1-R&D Systems assay.

In addition, multivariable regression was used to identify if the addition of other clinical or demographic variables previously reported to affect PAPP-A levels could further improve the prediction of ultrasound-determined GA (US GA) beyond that achieved by PAPP-A serum concentration levels as measured by the 4-Ansh Labs assay. Among the variables listed in Table E9, BMI and smoking status had significant p-values independent of 4-Ansh Labs-determined GA (smoking: p=0.007, partial r=0.167; BMI: p=0.001, partial r=0.202; and 4-Ansh Labs-determined GA: p<0.0001, partial r=0.934), with current tobacco use and higher BMI both associated with higher GA. The multivariable coefficient of determination was 0.880, a small increase compared to that achieved by 4-Ansh Labs-determined GA alone (0.872). Variables excluded from the model based on p>0.1 were maternal age, gravidity, alcohol use, uterine anomalies, and hypertension. The equation modeling US GA that includes the significant variables is as follows:

US GA=−5.505+[0.960*4-Ansh Labs GA (calculated as above using the fitted log-log regression equation)]+[0.258*BMI]+[3.523*1 (for smoking) or 0 (for non-smoking)].

In terms of accuracy of prediction of pregnancies over 70 days of gestation, adding any of these variables to the predictive equation of 4-Ansh Labs GA did not significantly increase AUROC (difference between areas=0.0002, p=0.899). With the 1-R&D Systems assay, no demographic variable had the ability to improve its predictive value, which was significantly lower than that achieved by 4-Ansh Labs calculated GA (R&D AUROC: 0.875, p<0.0001).

### D. Conclusions

These results corroborate the findings described in Example 2, indicating a strong ability of serum PAPP-A to distinguish between pregnancies above and below 70-days gestation. Specifically, GA can be predicted with high sensitivity and specificity broadly across an intended-use population of patients seeking medical abortion, at least some of said patients having relatively low GAs (e.g., below 70 days), using an immunoassay (e.g., 4-Ansh Labs) that detects homodimeric PAPP-A and the heterotetrameric PAPP-A/proMBP complex. This study also validates the finding that by reducing the dilution level of serum samples, the range of this immunoassay can be expanded in order to measure PAPP-A concentration levels in more patients of the intended-use population (e.g., having low GA), with the modified dilution protocols developed herein expanding the capacity to predict GA to almost all of the intended-use population. In comparison, unmodified dilution protocols could be used in less than half the intended-use population. In addition, the 1-R&D Systems assay could not be used to assess serum PAPP-A concentrations in more than half of the samples from the intended-use population, and biochemical GA based on the 1-R&D Systems assay could not be accurately determined below a GA of 74 days.

In addition, these results that additional clinical and demographic variables can be used to predict GA in the intended-use population. Specifically, smoking habits and BMI were also predictive of GA above and below 70 days in patients seeking medical abortion, with the inclusion of smoking habits and BMI in multivariate analysis with PAPP-A-determined GA accounting for more variation in US GA than PAPP-A determined GA alone.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

What is claimed:

1. A method of detecting PAPP-A in a biological sample from a pregnant subject, comprising:
(a) preparing a test sample from a pregnant female subject, the preparing comprising:
(i) obtaining a biological sample from the pregnant female subject, wherein the obtained biological sample is a whole blood, serum, or plasma sample and wherein the obtained biological sample has a volume between or between about 0.5 μL and 10 μL, inclusive; and
(ii) diluting the volume of the obtained biological sample in a sample diluent between or between about 2-fold and 100-fold, inclusive, thereby preparing the test sample; and
(b) detecting in the test sample one or more PAPP-A proteoforms using an immunoassay, said one or more PAPP-A proteoforms comprising homodimeric PAPP-A and/or a heterotetrameric PAPP-A/proMBP complex, wherein the immunoassay comprises:
(i) contacting the test sample with an antibody capable of specifically binding to (1) homodimeric PAPP-A and (2) the heterotetrameric PAPP-A/proMBP complex under conditions to form a complex comprising the antibody and the one or more PAPP-A proteoforms; and
(ii) detecting the complex comprising the antibody and the one or more PAPP-A proteoforms.

2. The method of claim 1, wherein when the biological sample is obtained, the gestational age (GA) of the pregnant female subject is suspected to be between or between about 5 weeks and 40 weeks, between or between about 5 weeks and 30 weeks, between or between about 5 weeks and 20 weeks, or between or between about 5 weeks and 15 weeks, each inclusive.

3. The method of claim 1, wherein when the biological sample is obtained, the GA of the pregnant female subject is suspected to be between or between about 5 weeks and 10 weeks, inclusive.

4. The method of any of claims 1-3, wherein the immunoassay is a colorimetric assay.

5. The method of any of claims 1-4, wherein the immunoassay is a solid-phase immunoassay, optionally wherein the antibody is immobilized on a solid support.

6. The method of any of claims 1-5, wherein the immunoassay is an Enzyme linked immunosorbent assay (ELISA), optionally a sandwich ELISA.

7. The method of any of claims 1-5, wherein the immunoassay is a lateral flow assay.

8. The method of any of claims 1-7, wherein the antibody is capable of specifically binding to (1) homodimeric PAPP-A and (2) the heterotetrameric PAPP-A/proMBP complex equimolarly or about equimolarly.

9. The method of any of claims 1-8, wherein the antibody is a capture antibody and the complex is a first complex comprising the capture antibody and the one or more PAPP-A proteoforms, and wherein:
prior to the detecting, the immunoassay further comprises contacting the first complex comprising the capture antibody and the one or more PAPP-A proteoforms with a detection antibody capable of specifically binding to (1) homodimeric PAPP-A and (2) the heterotetrameric PAPP-A/proMBP complex under conditions to form a second complex comprising the capture antibody, the one or more PAPP-A proteoforms, and the detection antibody; and
the detecting comprises detecting the second complex.

10. The method of any of claims 1-8, wherein the antibody is a detection antibody and the complex is a first complex comprising the detection antibody and the one or more PAPP-A proteoforms, and wherein:
prior to the detecting, the immunoassay further comprises contacting the first complex comprising the detection antibody and the one or more PAPP-A proteoforms with a capture antibody capable of specifically binding to (1) homodimeric PAPP-A and (2) the heterotetrameric PAPP-A/proMBP complex under conditions to form a second complex comprising the capture antibody, the one or more PAPP-A proteoforms, and the detection antibody; and
the detecting comprises detecting the second complex.

11. The method of claim 9 or claim 10, wherein the detection antibody is conjugated to a detectable label capable of producing a detectable signal, and the detecting the second complex is by assessing the degree of the detectable signal produced by the detectable label.

12. The method of any of claims 1-11, further comprising determining the concentration of PAPP-A in the obtained biological sample by comparison of the degree of the detectable signal assessed from the test sample to a standard curve.

13. A method of detecting PAPP-A in a biological sample from a pregnant subject, comprising:
(a) preparing a test sample from a pregnant female subject, the preparing comprising:
(i) obtaining a biological sample from the pregnant female subject, wherein the obtained biological sample is a whole blood, serum, or plasma sample and wherein the obtained biological sample has a volume between or between about 0.5 μL and 10 μL, inclusive; and
(ii) diluting the volume of the obtained biological sample in a sample diluent between or between about 2-fold and 100-fold, inclusive, thereby preparing the test sample; and
(b) detecting in the test sample one or more PAPP-A proteoforms using an immunoassay, said one or more PAPP-A proteoforms comprising homodimeric PAPP-A and/or a heterotetrameric PAPP-A/proMBP complex, wherein the immunoassay comprises:
(i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and
(ii) assessing the degree of the detectable signal produced by the detectable label.

14. The method of claim 13, wherein the contacting of the test sample with the capture antibody and the detection antibody is carried out simultaneously.

15. The method of claim 13, wherein the contacting of the test sample with the capture antibody and the detection antibody is carried out sequentially, in either order.

16. The method of claim 13 or claim 15, wherein the test sample is contacted with the capture antibody prior to being contacted with the detection antibody, wherein:
the test sample is contacted with the capture antibody under conditions to form a first complex comprising the capture antibody and the one or more PAPP-A proteoforms; and
the first complex is contacted with the detection antibody under conditions to form a second complex comprising the capture antibody, the one or more PAPP-A proteoforms, and the detection antibody.

17. The method of claim 13 or claim 15, wherein the test sample is contacted with the detection antibody prior to being contacted with the capture antibody, wherein:
the test sample is contacted with the detection antibody under conditions to form a first complex comprising the detection antibody and the one or more PAPP-A proteoforms; and
the first complex is contacted with the capture antibody under conditions to form a second complex comprising the capture antibody, the one or more PAPP-A proteoforms, and the detection antibody.

18. The method of claim any of claims 13-17, wherein when the biological sample is obtained, the gestational age (GA) of the pregnant female subject is suspected to be between or between about 5 weeks and 40 weeks, between or between about 5 weeks and 30 weeks, or between or between about 5 weeks and 20 weeks, between or between about 5 weeks and 15 weeks, or between or between about 5 weeks and 10 weeks, each inclusive.

19. The method of any of claims 13-18, wherein the immunoassay is a colorimetric assay.

20. The method of any of claims 13-19, wherein the immunoassay is a solid-phase immunoassay.

21. The method of any of claims 13-20, wherein the immunoassay is an Enzyme linked immunosorbent assay (ELISA), optionally a sandwich ELISA.

22. The method of any of claims 13-20, wherein the immunoassay is a lateral flow assay.

23. The method of any of claims 11-22, the method further comprising classifying the GA of the pregnant female subject based on the degree of the detectable signal assessed from the test sample.

24. The method of any of claims 11-22, further comprising determining the concentration of PAPP-A in the obtained biological sample by comparison of the degree of the detectable signal measured from the test sample to a standard curve.

25. The method of claim 12 or claim 24, the method further comprising classifying the GA of the pregnant female subject based on the concentration of PAPP-A in the obtained biological sample.

26. A method for classifying the gestational age (GA) of a pregnancy, comprising:
(a) detecting PAPP-A in a biological sample obtained from a pregnant female subject according to the method of any of claims 11-25;
(b) comparing the degree of the detectable signal assessed from the test sample to the degree of a detectable signal assessed using the immunoassay from a reference PAPP-A sample, wherein the reference PAPP-A sample comprises (1) homodimeric PAPP-A and/or (2) the heterotetrameric PAPP-A/proMBP complex, and wherein the concentration of the reference PAPP-A sample is a predetermined concentration associated with a predetermined GA cutpoint using the immunoassay; and
(c) classifying:
the GA of the pregnancy as less than the predetermined GA cutpoint if the degree of the detectable signal assessed from the test sample is lower than the degree of the detectable signal assessed from the reference sample; or
the GA of the pregnancy as greater than or equal to the predetermined GA cutpoint if the degree of the detectable signal assessed from the test sample is higher than or equal to the degree of the detectable signal assessed from the reference sample.

27. The method of claim 26, wherein the reference PAPP-A sample comprises the heterotetrameric PAPP-A/proMBP complex.

28. A method for classifying the gestational age (GA) of a pregnancy, comprising:
(a) detecting PAPP-A in a biological sample obtained from a pregnant female subject using an immunoassay, wherein:
the biological sample is a whole blood, serum, or plasma sample; and
the immunoassay comprises detecting one or more PAPP-A proteoforms in a test sample derived from the biological sample, said one or more PAPP-A proteoforms comprising homodimeric PAPP-A or a heterotetrameric PAPP-A/proMBP complex and said detecting comprising:
(i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and
(ii) assessing the degree of the detectable signal produced by the detectable label;
(b) comparing the degree of the detectable signal assessed from the test sample to the degree of a detectable signal measured using the immunoassay from a reference PAPP-A sample, wherein the reference PAPP-A sample comprises the heterotetrameric PAPP-A/proMBP complex, and wherein the concentration of the reference PAPP-A sample is a predetermined concentration associated with a predetermined GA cutpoint; and
(c) classifying:
the GA of the pregnancy as less than the predetermined GA cutpoint if the degree of the detectable signal assessed from the test sample is lower than the degree of the detectable signal assessed from the reference sample; or
the GA of the pregnancy as greater than or equal to the predetermined GA cutpoint if the degree of the detectable signal assessed from the test sample is higher than or equal to the degree of the detectable signal assessed from the reference sample.

29. The method of claim 27 or claim 28, wherein the reference PAPP-A sample further comprises homodimeric PAPP-A.

30. The method of any of claims 27-29, wherein the reference PAPP-A sample consists essentially of the heterotetrameric PAPP-A/proMBP complex.

31. A method for classifying the gestational age (GA) of a pregnancy, comprising:
(a) determining the concentration of PAPP-A in a biological sample obtained from a pregnant female subject according to the method of claim 12 or claim 24;
(b) comparing the concentration of PAPP-A in the obtained biological sample to a predetermined concentration of PAPP-A, wherein the predetermined concentration is associated with a predetermined GA cutpoint using the immunoassay; and
(c) classifying:
the GA of the pregnancy as less than a predetermined GA cutpoint if the concentration of PAPP-A in the obtained biological sample is lower than the predetermined concentration; or
the GA of the pregnancy as greater than or equal to the predetermined GA cutpoint if the concentration of PAPP-A in the obtained biological sample is higher than or equal to the predetermine concentration.

32. The method of any of claims 26-31, wherein the predetermined GA cutpoint is a timepoint between or between about 5 weeks and 40 weeks, between or between about 5 weeks and 30 weeks, or between or between about 5 weeks and 20 weeks, each inclusive.

33. The method of any of claims 26-31, wherein the predetermined GA cutpoint is a timepoint between or between about 5 weeks and 15 weeks, inclusive.

34. The method of any of claims 26-31, wherein the predetermined GA cutpoint is a timepoint between or between about 60 days and 140 days, inclusive.

35. The method of any of claims 26-31, wherein the predetermined GA cutpoint is a timepoint between or between about 56 days and 84 days, inclusive.

36. The method of any of claims 26-31, wherein the predetermined GA cutpoint is or is about 56 days, 63 days, 70 days, 77 days, or 84 days.

37. The method of any of claims 26-31, wherein the predetermined GA cutpoint is a timepoint between or between about 63 days and 77 days, inclusive.

38. The method of any of claims 26-31, wherein the predetermined GA cutpoint is or is about 63 days, 70 days, or 77 days.

39. The method of any of claims 26-31, wherein the predetermined GA cutpoint is or is about 56 days.

40. The method of any of claims 26-33, 35, 36, and 39, wherein the predetermined concentration is between or between about 1 ng/mL and 70 ng/mL, inclusive.

41. The method of any of claims 26-33, 35, 36, and 39, wherein the predetermined concentration is between or between 5 ng/mL and 55 ng/mL, inclusive.

42. The method of any of claims 26-31, wherein the predetermined GA cutpoint is or is about 63 days.

43. The method of any of claims 26-38 and 42, wherein the predetermined concentration is between or between about 25 ng/mL and 150 ng/mL, inclusive.

44. The method of any of claims 26-38 and 42, wherein the predetermined concentration is between or between 40 ng/mL and 130 ng/mL, inclusive.

45. The method of any of claims 26-31, wherein the predetermined GA cutpoint is or is about 70 days.

46. The method of any of claims 26-38 and 45, wherein the predetermined concentration is between or between about 20 ng/mL and 200 ng/mL, inclusive.

47. The method of any of claims 26-38 and 45, wherein the predetermined concentration is between or between 30 ng/mL and 150 ng/mL, inclusive.

48. The method of any of claims 26-31, wherein the predetermined GA cutpoint is or is about 77 days.

49. The method of any of claims 26-38 and 48, wherein the predetermined concentration is between or between about 80 ng/mL and 220 ng/mL, inclusive.

50. The method of any of claims 26-38 and 48, wherein the predetermined concentration is between or between 90 ng/mL and 215 ng/mL, inclusive.

51. The method of any of claims 26-31, wherein the predetermined GA cutpoint is or is about 84 days.

52. The method of any of claims 26-36 and 51, wherein the predetermined concentration is between or between about 160 ng/mL and 480 ng/mL, inclusive.

53. The method of any of claims 26-36 and 51, wherein the predetermined concentration is between or between 170 ng/mL and 470 ng/mL, inclusive.

54. The method of any of claims 26-31, wherein the predetermined GA cutpoint is a timepoint between or between about 98 days and 112 days, inclusive.

55. The method of any of claims 26-31, wherein the predetermined GA cutpoint is or is about 98 days, 105 days, or 112 days.

56. The method of any of claims 26-31, wherein the predetermined GA cutpoint is or is about 98 days.

57. The method of any of claims 26-34 and 56, wherein the predetermined concentration is between or between about 550 ng/mL and 1240 ng/mL, inclusive.

58. The method of any of claims 26-34 and 56, wherein the predetermined concentration is between or between 565 ng/mL and 1230 ng/mL, inclusive.

59. The method of any of claims 26-31, wherein the predetermined GA cutpoint is or is about 105 days.

60. The method of any of claims 26-34 and 59, wherein the predetermined concentration is between or between about 1440 ng/mL and 1490 ng/mL, inclusive.

61. The method of any of claims 26-34 and 59, wherein the predetermined concentration is between or between 1450 ng/mL and 1475 ng/mL, inclusive.

62. The method of any of claims 26-31, wherein the predetermined GA cutpoint is or is about 112 days.

63. The method of any of claims 26-32 and 62, wherein the predetermined concentration is between or between about 1440 ng/mL and 1490 ng/mL, inclusive.

64. The method of any of claims 26-32 and 62, wherein the predetermined concentration is between or between 1450 ng/mL and 1475 ng/mL, inclusive.

65. The method of any of claims 26-31, wherein the predetermined GA cutpoint is or is about 168 days.

66. The method of any of claims 26-32 and 65, wherein the predetermined concentration is between or between about 3500 ng/mL and 4500 ng/mL, inclusive.

67. The method of any of claims 26-32 and 65, wherein the predetermined concentration is between or between 4000 ng/mL and 4200 ng/mL, inclusive.

68. The method of any of claims 23 and 25-67, wherein the classifying is performed with greater than or greater than about 80%, 85%, or 90% sensitivity and/or greater than or greater than about 80%, 85%, or 90% specificity.

69. The method of any of claims 23 and 25-68, wherein the classifying is further based on whether the subject was using tobacco products at the time the biological sample was obtained, wherein the GA of the pregnancy is more likely to be classified as greater than or equal to the predetermined GA cutpoint if the subject was using tobacco products.

70. The method of any of claims 23 and 25-60, wherein the classifying is further based on the body mass index (BMI) of the subject at the time the biological sample was obtained, wherein the GA of the pregnancy is more likely to be classified as greater than or equal to the predetermined GA cutpoint if the BMI of the subject is higher.

71. A method for screening a pregnant subject for a prenatal care or prenatal clinical treatment, comprising:
(a) classifying the gestational age (GA) of a pregnancy according to the method of any of claims 23 and 25-70; and
(b) based on the classifying, (i) selecting the pregnant female subject as eligible for a prenatal care or prenatal clinical treatment if the GA of the pregnancy is classified as less than the predetermined GA cutpoint; or (ii) selecting the pregnant female subject as not eligible for the prenatal care or prenatal clinical treatment or as a candidate for further assessment for the prenatal care or prenatal clinical treatment if the GA of the pregnancy is classified as greater than or equal to the predetermined GA cutpoint.

72. A method for performing a prenatal care or prenatal clinical treatment on a pregnant subject, comprising performing a prenatal care or prenatal clinical treatment on a pregnant female subject selected as eligible for the prenatal care or prenatal clinical treatment according to the method of claim 71.

73. A method for determining the gestational age (GA) of a pregnancy, comprising:
(a) detecting PAPP-A in a biological sample obtained from a pregnant female subject according to the method of any of claims 11-25; and
(b) determining the GA of the pregnancy based on the degree of the detectable signal assessed from the test sample, wherein the determining comprises providing the degree of the detectable signal assessed from the test sample as input to a process that uses the degree of the detectable signal measured from the test sample to predict GA.

74. A method for determining the gestational age (GA) of a pregnancy, comprising:
(a) detecting PAPP-A in a biological sample obtained from a pregnant female subject using an immunoassay, wherein:
the biological sample is a whole blood, serum, or plasma sample; and
the immunoassay comprises detecting one or more PAPP-A proteoforms in a test sample derived from the biological sample, said one or more PAPP-A proteoforms comprising homodimeric PAPP-A or a heterotetrameric PAPP-A/proMBP complex, said detecting comprising:
(i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and
(ii) measuring the degree of the detectable signal produced by the detectable label; and
(b) determining the GA of the pregnancy based on the degree of the detectable signal assessed from the test sample, wherein the determining comprises providing the degree of the detectable signal assessed from the test sample as input to a process that uses the degree of the detectable signal assessed from the test sample to predict GA.

75. A method for determining the gestational age (GA) of a pregnancy, comprising:
(a) measuring the concentration of PAPP-A in a biological sample obtained from a pregnant female subject according to the method of claim 12 or claim 24; and
(b) determining the GA of the pregnancy based on the concentration of PAPP-A in the obtained biological sample, wherein the determining comprises providing the concentration of PAPP-A in the obtained biological sample as input to a process that uses PAPP-A concentration as a continuous predictor of GA.

76. A method for determining the gestational age (GA) of a pregnancy, comprising:
(a) determining the concentration of PAPP-A in a biological sample obtained from a pregnant female subject using an immunoassay, wherein:
the biological sample is a whole blood, serum, or plasma sample; and
the immunoassay comprises detecting one or more PAPP-A proteoforms in a test sample derived from the biological sample, said one or more PAPP-A proteoforms comprising homodimeric PAPP-A or a heterotetrameric PAPP-A/proMBP complex, said detecting comprising:
(i) contacting the test sample with a capture antibody and a detection antibody, wherein the capture antibody and the detection antibody are independently capable of specifically binding to (1) homodimeric PAPP-A and (2) a heterotetrameric PAPP-A/proMBP complex, and wherein the contacting is carried out under conditions to form a complex comprising the capture antibody, the one or more PAPP-A proteoforms, and the detection antibody, wherein the detection antibody is conjugated to a detectable label that is capable of producing a detectable signal; and
(ii) assessing the degree of the detectable signal produced by the detectable label; and
(iii) determining the concentration of PAPP-A in the obtained biological sample by comparison of the degree of the detectable signal to a standard curve; and
(b) determining the GA of the pregnancy based on the concentration of PAPP-A in the obtained biological sample, wherein the determining comprises providing the concentration of PAPP-A in the obtained biological sample as input to a process that uses PAPP-A concentration as a continuous predictor of GAs.

77. The method of any of claims 73-76, wherein the contacting of the test sample with the capture antibody and the detection antibody is carried out simultaneously.

78. The method of any of claims 73-76, wherein the contacting of the test sample with the capture antibody and the detection antibody is carried out sequentially in either order.

79. The method of any of claims 73-76 and 78, wherein the test sample is contacted with the capture antibody prior to being contacted with the detection antibody, wherein:
the test sample is contacted with the capture antibody under conditions to form a first complex comprising the capture antibody and the one or more PAPP-A proteoforms; and
the first complex is contacted with the detection antibody under conditions to form a second complex comprising the capture antibody, the one or more PAPP-A proteoforms, and the detection antibody.

80. The method of any of claims 73-76 and 78, wherein the test sample is contacted with the detection antibody prior to being contacted with the capture antibody, wherein:
the test sample is contacted with the detection antibody under conditions to form a first complex comprising the detection antibody and the one or more PAPP-A proteoforms; and
the first complex is contacted with the capture antibody under conditions to form a second complex comprising the capture antibody, the one or more PAPP-A proteoforms, and the detection antibody.

81. The method of any of claims 73-80, wherein when the biological sample is obtained, the gestational age (GA) of the pregnant female subject is suspected to be between or between about 5 weeks and 40 weeks, between or between about 5 weeks and 30 weeks, between or between about 5 weeks and 20 weeks, or between or between about 5 weeks and 15 weeks, each inclusive.

82. The method of any of claims 73-80, wherein when the biological sample is obtained, the GA of the pregnant female subject is suspected to be between or between about 5 weeks and 10 weeks, inclusive.

83. The method of any of claims 73-82, wherein the immunoassay is a colorimetric assay.

84. The method of any of claims 73-83, wherein the immunoassay is a solid-phase immunoassay.

85. The method of any of claims 73-84, wherein the immunoassay is an Enzyme linked immunosorbent assay (ELISA), optionally a sandwich ELISA.

86. The method of any of claims 73-84, wherein the immunoassay is a lateral flow assay.

87. The method of any of claims 73-86, wherein the process comprises a regression model trained using GAs and PAPP-A concentrations from a plurality of pregnant female subjects.

88. The method of claim 87, wherein the regression model is a linear regression model, a piecewise linear model, a polynomial regression model, or a Bayesian model.

89. The method of any of claims 73-88, wherein the process predicts GAs between or between about 5 weeks and 40 weeks, between or between about 5 weeks and 30 weeks, between or between about 5 weeks and 20 weeks, between or between about 5 weeks and 15 weeks, or between or between about 10 weeks and 15 weeks, each inclusive.

90. The method of any of claims 73-89, wherein the determining further comprises providing whether or not the subject was using tobacco products at the time the biological sample was obtained as input to the process, and the process further uses whether the subject was using tobacco products as a predictor of GA.

91. The method of any of claims 73-90, wherein the determining further comprises providing the body mass index (BMI) of the subject at the time the biological sample was obtained as input to the process, and the process further uses the BMI as a predictor of GA.

92. A method of selecting a prenatal care or prenatal clinical treatment for a pregnant subject, comprising:
(a) determining the gestational age (GA) of a pregnancy according to the method of any of claims 73-91; and
(b) selecting a prenatal care or prenatal clinical treatment for the pregnant female subject based on the GA of the pregnancy.

93. A method for performing a prenatal care or prenatal clinical treatment on a pregnant subject, comprising performing a prenatal care or prenatal clinical treatment on a pregnant female subject, wherein the prenatal care or prenatal clinical treatment is selected according to the method of claim 92.

94. The method of any of claim 71, 72, 92, or 93, wherein the prenatal care or prenatal clinical treatment is a medical abortion or early aspiration; a decision about a medical abortion regimen; a decision about the risk of embryotoxicity; a clinical examination; a vaccination; a risk assessment; a fetal assessment; a blood assay; a urine assay; vitamin supplementation; a test for disease; education; counseling; or any combination of any of the foregoing.

95. The method of any of claim 71, 72, 92, or 93, wherein the prenatal care or prenatal clinical treatment is a medical abortion or early aspiration.

96. The method of any of claims 1-95, wherein the obtained biological sample has a volume between or between about 0.5 μL, and 10 μL, inclusive.

97. The method of any of claims 1-96, wherein the obtained biological sample has a volume between or between about 0.5 μL, and 5 μL, inclusive, optionally wherein the obtained biological sample has a volume of or of about 1 μL.

98. The method of any of claims 1-97, wherein the test sample is prepared by diluting the obtained biological sample with a sample diluent prior to the measuring.

99. The method of any of claims 1-97, further comprising diluting the obtained biological sample with a sample diluent prior to the measuring.

100. The method of claim 98 or claim 99, wherein the volume of the obtained biological sample is diluted between or between about 2-fold and 100-fold, inclusive.

101. The method of any of claims 1-100, wherein the volume of the obtained biological sample is diluted between or between about 2-fold and 75-fold, inclusive, optionally wherein the volume of the obtained biological sample is diluted or diluted about 5-fold or 50-fold.

102. The method of any of claims 1-101, wherein the sample diluent comprises one or both of buffered saline and a nonionic detergent.

103. The method of any of claims 1-102, wherein the obtained biological sample is a whole blood sample.

104. The method of any of claims 1-102, wherein the obtained biological sample is a serum sample.

105. The method of any of claims 1-104, further comprising obtaining the biological sample from the pregnant female subject.

106. The method of any of claims 9-105, wherein the capture antibody and/or the detection antibody is capable of specifically binding to (1) homodimeric PAPP-A and (2) the heterotetrameric PAPP-A/proMBP complex equimolarly or about equimolarly.

107. The method of any of claims 9-106, wherein the capture antibody is unlabeled.

108. The method of any of claims 9-107, wherein the capture antibody is immobilized on a solid support.

109. The method of any of claims 5-108, wherein the solid support is a bead, column, array, assay plate, microwell, cartridge, stick, filter, or strip.

110. The method of any of claims 5-109, wherein the solid support is formed of glass, polysaccharides, polyacrylamides, polystyrene, polyvinyl alcohol, nitrocellulose, cellulose, nylon, and/or silicones.

111. The method of any of claims 11-110, wherein the detectable label is or comprises horseradish peroxidase.

112. The method of any of claims 1-111, wherein the method is carried out using a point-of-care device.

113. The method of any of claims 1-112, wherein the method is carried out using a lateral flow stick.

114. The method of any of claims 1-111, wherein the method is carried out in a laboratory.

115. The method of any of claims 1-114, wherein the method is carried out without an ultrasound.

116. The method of any of claims 23-73 and 94-115, further comprising comparing the classified GA to a GA as determined by performing an ultrasound.

117. A device for carrying out the method of any of claims 1-116.

118. The device of claim 117 that is a hand-held device or a point-of-care device.

119. The device of claim 116 or claim 117, wherein the device comprises a solid support and one or more antibodies capable of independently specifically binding to (1) homodimeric PAPP-A and (2) a heterotetrameric PAPP-A/proMBP complex, wherein at least one of the one or more antibodies is immobilized on the solid support.

120. The device of claim 119, wherein the solid support is a bead, column, array, assay plate, microwell, cartridge, stick, filter, or strip.

121. The device of claim 119 or claim 120, wherein the solid support is formed of glass, polysaccharides, polyacrylamides, polystyrene, polyvinyl alcohol, nitrocellulose, cellulose, nylon, and/or silicones.

122. The device of any of claims 117-121 that is a lateral flow device.

123. A kit for carrying out the method of any of claims 1-116, comprising a reference PAPP-A sample and the device of any of claims 117-122, wherein:
the concentration of PAPP-A in the reference PAPP-A sample is for a predetermined GA cutpoint; and
the PAPP-A in the reference PAPP-A sample comprises heterotetrameric PAPP-A/proMBP complex.

124. The kit of claim 123, wherein the PAPP-A in the reference PAPP-A sample further comprises homodimeric PAPP-A.

125. The kit of claim 123 or claim 124, wherein the PAPP-A in the reference PAPP-A sample consists essentially of heterotetrameric PAPP-A/proMBP complex.

126. The kit of any of claims 123-125, wherein the predetermined GA cutpoint is a timepoint between or between about 5 weeks and 40 weeks, inclusive.

127. The kit of claim 123-125, wherein the predetermined GA cutpoint is a timepoint between or between about 64 days and 140 days, inclusive.

128. The kit of claim 123-125, wherein the predetermined GA cutpoint is or is about 63 days, 70 days, or 77 days.

129. The kit of any of claims 123-128, wherein the concentration of reference PAPP-A in the reference PAPP-A sample is between or between about 20 ng/mL and 200 ng/mL, inclusive.

130. The kit of any of claims 123-128, wherein the concentration of reference PAPP-A in the reference PAPP-A sample is between or between about 30 ng/mL and 150 ng/mL, inclusive.

131. The kit of any of claims 123-128, wherein the concentration of reference PAPP-A in the reference PAPP-A sample is or is about 133.2096 ng/mL.

132. The kit of any of claims 123-131, wherein the reference PAPP-A sample is a first reference PAPP-A sample and the predetermined GA cutpoint is a first predetermined GA cutpoint, and further comprising a second reference PAPP-A sample, wherein:
the concentration of PAPP-A in the second reference PAPP-A sample is for a second predetermined GA cutpoint that is later than the first predetermined GA cutpoint; and the PAPP-A in the second reference sample comprises (1) homodimeric PAPP-A and/or (2) the heterotetrameric PAPP-A/proMBP complex.

133. The kit of claim 132, wherein the second predetermined GA cutpoint is a timepoint between or between about 5 weeks and 40 weeks, inclusive.

134. The kit of claim 132, wherein the second predetermined GA cutpoint is a timepoint between or between about 64 days and 140 days, inclusive.

135. The kit of claim 132, wherein the second predetermined GA cutpoint is or is about 63 days, 70 days, or 77 days.

136. The kit of claim 132, wherein the second predetermined GA cutpoint is or is about 105 days.

137. The kit of claim 132, wherein the first predetermined GA cutpoint is or is about 63 days, and the second predetermined GA cutpoint is or is about 105 days+/−1 week, optionally about 98 days or 112 days.

138. The kit of claim 132, wherein the first predetermined GA cutpoint is or is about 70 days, and the second predetermined GA cutpoint is or is about 105 days+/−1 week, optionally about 98 days or 112 days.

139. The kit of claim 132, wherein the first predetermined GA cutpoint is or is about 77 days, and the second predetermined GA cutpoint is or is about 105 days+/−1 week, optionally about 98 days or 112 days.
